(19) United States
(12) Patent Application Publication
(10) Pub. No.: US 2024/0216163 A1
(43) Pub. Date: Jul. 4, 2024

(54) MECHANICALLY PROMOTING UPPER AIRWAY PATENCY

(71) Applicant: INSPIRE MEDICAL SYSTEMS, INC., Golden Valley, MN (US)

(72) Inventors: Wondimeneh Tesfayesus, New Brighton, MN (US); Kevin Verzal, Lino Lakes, MN (US); John Rondoni, Plymouth, MN (US)

(73) Assignee: INSPIRE MEDICAL SYSTEMS, INC., Golden Valley, MN (US)

(21) Appl. No.: 18/395,015

(22) Filed: Dec. 22, 2023

Related U.S. Application Data
(60) Provisional application No. 63/477,693, filed on Dec. 29, 2022.

(51) Int. Cl. A61F 5/56

(52) U.S. Cl. CPC A61F 5/56

(57) ABSTRACT

Examples are directed to an apparatus, device and/or method comprising promoting patency of an upper airway of a patient via mechanically maneuvering at least one of thyroid cartilage inferiorly, and hyoid bone anteriorly.

### CROSS-REFERENCE TO RELATED APPLICATIONS

This application claims the benefit of the filing date of U.S. Provisional Application Ser. No. 63/477,693, filed Dec. 29, 2022 and entitled “Mechanically Promoting Upper Airway Patency,” the entire teachings of which are incorporated herein by reference.

### BACKGROUND

Many patients benefit from therapy provided by an implantable medical device (IMD). For example, a portion of the population suffers from various forms of sleep disorder breathing (SDB). In some patients, external breathing therapy devices, surgical interventions, and/or electrical stimulation of nerves and/or muscles related to the upper airway patency may fail to treat the SDB.

### BRIEF DESCRIPTION OF THE DRAWINGS

FIG. 1 is a flow diagram schematically representing an example method comprising mechanically promoting upper airway patency.

FIGS. 2A-2F illustrate an example upper airway of a patient and different obstructions of the upper airway.

FIGS. 3A-3C illustrate an example implantable traction apparatus and placements for the implantable traction apparatus.

FIGS. 4A-4C illustrate different example implantable traction apparatus.

FIGS. 5A-5B are diagrams schematically representing deployment of example implantable traction apparatus.

FIGS. 6A-6C are diagrams schematically representing deployment of further example implantable traction apparatus.

FIGS. 7A-7C are diagrams schematically representing example methods.

FIGS. 8A-8C illustrate additional example implantable traction apparatus.

FIG. 9A is a flow diagram schematically representing an example method comprising selectively applying different care based on at least one sensed parameter and sensing of the at least one parameter.

FIG. 9B is a diagram schematically representing an example control portion and sensors.

FIG. 9C is a block diagram schematically representing an example sensing portion.

FIGS. 10A-10E illustrate example implantable medical devices which may be used in addition to the implantable traction apparatus.

FIG. 11A is a block diagram schematically representing an example control portion.

FIG. 11B is a diagram schematically illustrating at least some example arrangements of a control portion.

FIG. 12 is a block diagram schematically representing a user interface.

FIG. 13 is a block diagram which schematically represents some example implementations by which an implantable device may communicate wirelessly with external circuitry outside the patient.

FIG. 14 is a block diagram schematically representing a care engine of a control portion.

FIG. 15 is a diagram schematically representing a patient's body, implantable components, and/or external elements of an example device and/or for use in an example method.

FIGS. 16A-16D are block diagrams schematically representing example devices and/or example methods relating to collapse patterns associated with upper airway patency.

FIGS. 17 and 18 each are a block diagram schematically representing an example care engine and control portion, respectively.

FIG. 19 is a block diagram schematically representing an example user interface.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to apparatuses and devices for diagnosis, therapy and/or other care of medical conditions which may relate to upper airway patency. At least some examples may comprise an implantable traction apparatus, device, and/or methods comprising use of the implantable traction apparatus and/or device in order to increase or maintain upper airway patency. At least some of the examples of the present disclosure may be employed to treat sleep disordered breathing (SDB), which may comprise obstructive sleep apnea (OSA) and/or other types of sleep disordered breathing.

SDB may be treated using a variety of different techniques. In some instances, external breathing therapy devices, such as a continuous positive airway pressure (CPAP) machine or other devices which provide air pressure to the patient during sleep, are used to treat patients. However, such external breathing devices may not work for all patients and may be bothersome to the patient, resulting in reduced use. For example, external breathing devices may not work for patients with a deviated septum, enlarged turbinates, and/or that have particular allergies, among other conditions. In other instances, a patient may have difficulty fitting the device, may not tolerate the forced air well, and/or may have other unpleasant outcomes, such as dry nose, feeling of claustrophobia, leaking masks, difficulty sleeping, and other side effects which cause the patient to not use the device. Such patients may sometimes be referred to as being non-compliant or non-adherent because they fail to comply with the prescribed therapy.

For some patients and types of SDB, surgical interventions may be used to improve symptoms, such as uvulopalatopharyngoplasty, lateral pharyngoplasty, lingual tonsillectomy, and tongue reduction surgery, among other procedures. However, such surgical interventions have a mixed record of success for many cases of SDB.

On the other hand, for most patients exhibiting moderate and severe OSA, great success has been found with the use of some types of implantable neurostimulation devices that provide electrical stimulation to nerves and/or muscles promoting upper airway patency, sometimes herein referred to as upper airway patency-related tissue, nerves, and/or muscles. Nevertheless, a small percentage of patients may not respond to such neurostimulation therapy and therefore may sometimes be referred to as “non-responders”.

At least some examples of the present disclosure are directed to mechanical devices, apparatuses, and methods for stretching upper airway inferiorly to stiffen and reduce collapsibility using the mechanical devices and/or apparatuses.

At least some examples are directed to methods, apparatus, and/or devices involving mechanically maneuvering at least one of thyroid cartilage and a hyoid bone of a patient. Among other potential therapies, such mechanical maneuvering may be used to treat SDB patients which are non-responders to, and/or which are non-compliant with, other types of SDB treatment. In some such examples, the mechanical maneuvering may comprise maneuvering the thyroid cartilage inferiorly and/or the hyoid bone anteriorly (and/or variations and combinations thereof), which may act to displace tissue at least partially forming at least the oropharynx portion of the upper airway in order to reduce extraluminal tissue pressure that may otherwise crowd or reduce upper airway patency, specifically patency within and through the oropharynx portion of the upper airway.

In some examples, the mechanical maneuvering of the thyroid cartilage and/or hyoid bone may be selectively applied based on at least one sensed parameter. In many patients, sleep causes or results in the relaxation of muscles associated with upper airway patency, sometimes herein referred to as “upper airway patency-related muscles.” Sleep also may cause, or result in, other changes that lead to collapse of structures around the upper airway, which may contribute to obstruction of air passage through the upper airway during breathing. In some examples, the mechanical maneuvering may be applied in response to the patient sleeping and/or timed in relation to a respiration waveform the patient.

By timing the mechanical maneuvering relative to the respiration waveform and while the patient is sleeping, the mechanical maneuvering of thyroid cartilage and/or hyoid bone may mimic at least some aspects of natural activity of the upper airway patency-related muscles in a manner which promotes patency of at least the oropharynx during at least an inspiratory phase of respiration, which may counteract the tendency of the upper airway patency-related muscles to relax during sleep in a manner resulting in oropharyngeal narrowing or obstruction in some patients. In various examples, the mechanical maneuvering may be selectively applied in response to detecting obstruction of the upper airway and/or in response to unfavorable displacement of the thyroid cartilage and/or hyoid bone which may inhibit patency of the upper airway (and particularly including the oropharynx).

In some examples, the mechanical maneuvering may be applied without sensing for obstructions of the upper airway including, but not limited to, the oropharynx. In such examples, the mechanical maneuvering may be applied also without sensing, and/or otherwise receiving sensed physiologic information such as, but not limited to, respiration waveform information of the patient. However, in some examples, the mechanical maneuvering may be applied without sensing for obstructions of the upper airway including, but not limited to, the oropharynx but while still sensing (and/or otherwise receiving) respiration waveform information, which may be used for timing application of the mechanical maneuvering. It will be understood that sensing for obstructions of the upper airway generally, and/or of the oropharynx specifically, may include (but is not limited to) sensing for OSA events.

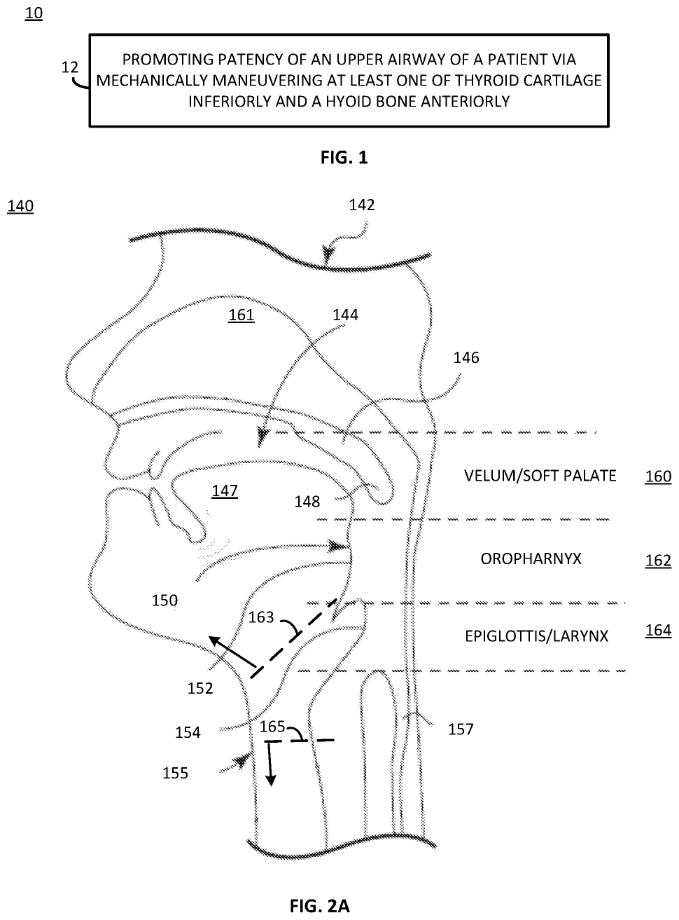

These examples, and additional examples, are described in connection with at least FIGS. 1-19.

FIG. 1 is a flow diagram schematically representing an example method comprising mechanically maneuvering at least one of thyroid cartilage and a hyoid bone. The method 10 comprises promoting patency of an upper airway of a patient via mechanically maneuvering at least one of thyroid cartilage inferiorly and a hyoid bone anteriorly, as shown at 12 in FIG. 1. In some examples, the method 10 may promote patency of at least an oropharynx portion of the upper airway. Among other portions of the upper airway which may be affected, the mechanical maneuvering may cause an elongation of (e.g., stretching, pulling tension on) at least the oropharynx portion of the upper airway in a manner which causes an increase of, and/or maintaining, patency of at least the oropharynx of the upper airway. For example, the mechanical maneuvering may stretch the upper airway inferiorly to stiffen the upper airway and reduce collapsibility, and which may be performed using the devices and/or apparatuses as further described herein.

As further described herein, the upper airway includes and/or refers to air-conducting passages of the respiratory system that extend to the larynx from the openings of the nose and from the lips through the mouth. The oropharynx portion of the upper airway may include at least a portion (or all) of the oropharynx that extends approximately from the tip of the soft palate along the base of the tongue until reaching approximately the tip region of the epiglottis. The thyroid cartilage includes and/or refers to tissue in and around at least part of the trachea that contains the larynx, and which is inferior to the hyoid bone. The hyoid bone is a bone positioned in an anterior midline of the neck between the mandible and thyroid cartilage.

In some examples, mechanically maneuvering the at least one of the thyroid cartilage inferiorly and the hyoid bone anteriorly also may displace tissue (e.g., adipose) within and/or at least partially forming the walls of the oropharynx of the upper airway, sometimes herein referred to as the oropharynx walls or pharyngeal walls (with oropharynx walls being a subset of pharyngeal walls). The displacement of this tissue may reduce extraluminal tissue space in the walls at least partially defining the oropharynx, which reduces extraluminal tissue pressure which would otherwise force the walls of the oropharynx inward to reduce patency. However, by reducing extraluminal tissue pressure, upper airway patency (e.g., oropharyngeal patency) is increased or at least maintained, thereby reducing or preventing SDB.

As further illustrated herein, in some examples the method 10 may be performed using an implantable traction apparatus which provides the mechanical maneuvering action. An example implantable traction apparatus may be used for treating SDB, such as for sleep apnea. Sleep apnea generally refers to the cessation of breathing during sleep. One type of sleep apnea, referred to as OSA, may be characterized by repetitive pauses in breathing during sleep due to the obstruction and/or collapse of the upper airway, and is usually accompanied by a reduction in blood oxygenation saturation.

The mechanical maneuvering of the thyroid cartilage and/or hyoid bone via the implantable traction apparatus is an example of treatment for OSA or other types of SDB. The mechanical maneuvering may mimic action of at least some behavior of the upper airway patency-related muscles and/or otherwise displaces tissue at least partially defining walls of the upper airway (e.g., oropharyngeal walls) to maintain patency of the upper airway, such as at least the oropharynx portion. As described above, some patients may not be compliant with and/or not respond well to various types of treatment for SDB, such as external breathing therapy devices, surgical approaches, and/or delivery of electrical stimulation to the hypoglossal nerve, located in the neck region under the chin. Accordingly, in some examples, as further described herein, the mechanical maneuvering may be applied in combination with another treatment, such as the use of external breathing therapy devices and/or electrical stimulation of nerves and/or muscle. In treatment of sleep apnea, increased respiratory effort resulting from the difficulty in breathing through an obstructed airway is avoided by mechanically maneuvering the thyroid cartilage and/or hyoid bone to hold the airway open during at least a portion of the inspiratory phase of breathing. In some examples, the mechanically maneuvering may be timed relative to (e.g., timed to coincide with at least a portion of) breathing (e.g., an inspiratory phase of each respiratory cycle).

However, examples are not so limited and, as noted above, some examples may include a static mechanical maneuvering of the thyroid cartilage and/or hyoid bone, e.g., a mechanical maneuvering which is not timed relative to the portion of the respiratory cycle.

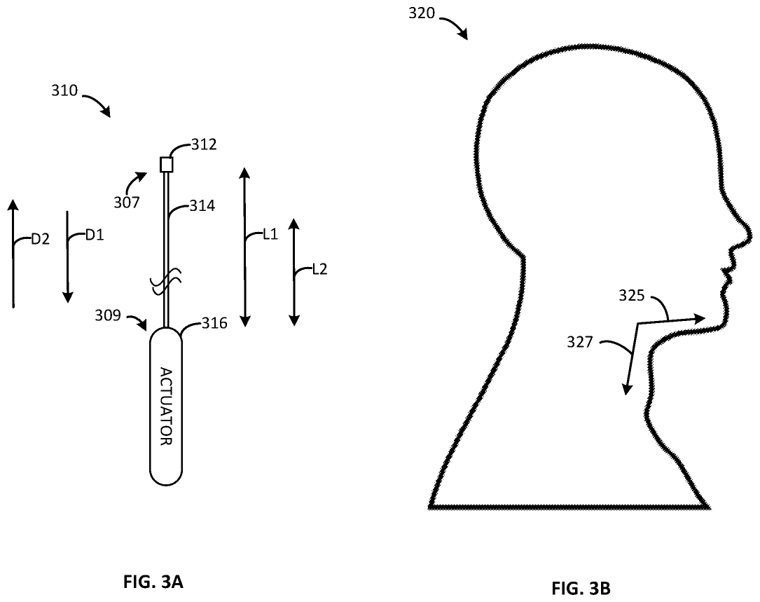

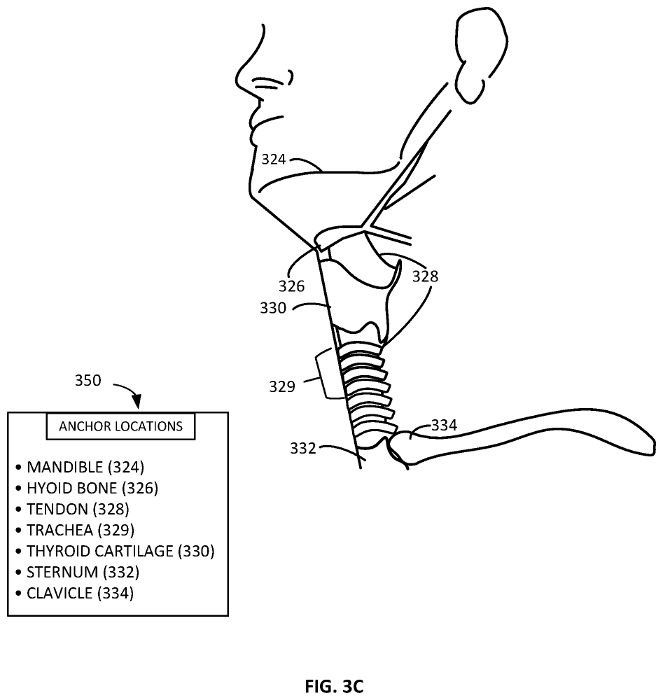

As further described herein, the method 10 may comprise a number of additional steps and/or variations, such as those illustrated in connection with FIGS. 3A-6C, 7A-7C, 8A-8C, 9A-9C and 10A-19.

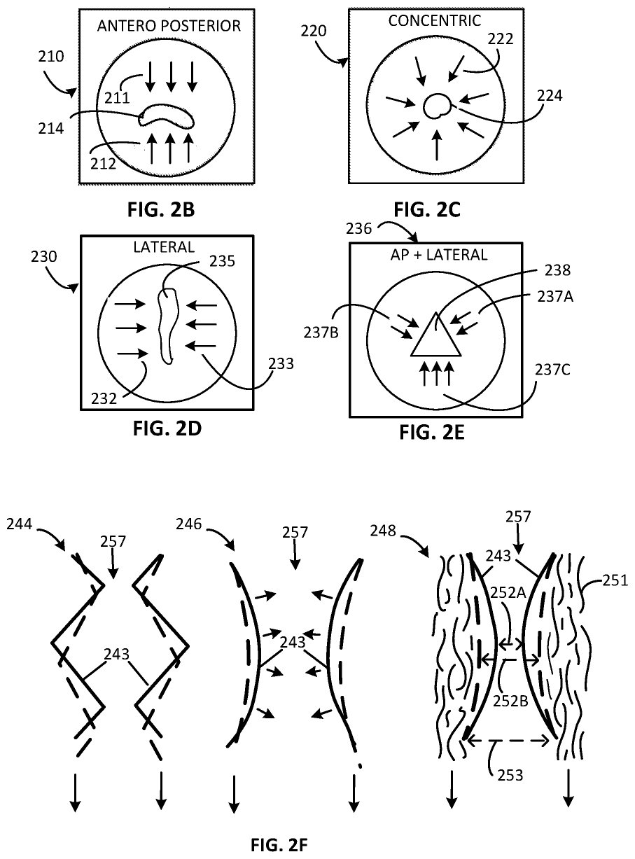

FIGS. 2A-2F illustrate an example upper airway of a patient and different obstructions of the upper airway.

FIG. 2A is a side view schematically illustrating an example upper airway of a patient. FIG. 2A is a diagram 140 of a side sectional view (cross hatching omitted for illustrative clarity) of a head and neck region 142 of a patient. In particular, an upper airway portion 150 extends from the mouth 144 to a neck portion 155. The upper airway portion 150 includes a velum (soft palate) portion (or region) 160, an oropharynx portion (or region) 162, and an epiglottis-larynx portion (or region) 164. The velum (soft palate) portion 160 includes an area extending below sinus 161, and includes the soft palate 146 approximately to the point at which tip 148 of the soft palate 146 meets a portion of tongue 147 at the back of the mouth 144. The oropharynx portion 162 extends approximately from the tip of the soft palate 146 along the base 152 of the tongue 147 until reaching approximately the tip region of the epiglottis 154. The epiglottis-larynx portion 164 extends approximately from the tip of the epiglottis 154 downwardly to a point above the esophagus 157.

FIG. 2A further illustrates relative location of the hyoid bone 163 and thyroid cartilage 165, as illustrated by dashed lines and with the arrows illustrating the direction of the mechanically maneuvering of the hyoid bone 163 and thyroid cartilage 165, in accordance with some examples of the present disclosure.

As shown, the hyoid bone 163 relates to the base 152 of the tongue 147 (e.g., genioglossus muscle) such that pulling hyoid bone 163 anteriorly, as shown by the arrow, may pull the base 152 of the tongue 147 forward (and/or also cause the middle pharyngeal constrictor muscle to stretch) which effectively moves the anterior “wall” of the oropharynx anteriorly, and therefore increases cross-sectional area (e.g., patency) of oropharynx.

In some examples, pulling the hyoid bone 163 anteriorly may elongate (e.g., stretch) at least one at least one pharyngeal constrictor muscle, such as the middle, inferior, and/or superior pharyngeal constrictor muscles. For example, the middle pharyngeal constrictor muscle may attach to the hyoid bone 163 and displacement of the hyoid bone 163 anteriorly may cause the middle pharyngeal constrictor muscle to elongate (e.g., stretch) and increase airway patency in at least the oropharynx portion 162. In some examples, elongating (e.g., stretching) the at least one pharyngeal constrictor muscle may stiffen the upper airway (e.g., increases pharyngeal muscle tone) and reduce collapsibility of the upper airway. In some examples, the hyoid bone 163 may not move in a purely anterior-posterior orientation. As such, as used herein, the hyoid bone 163 being moved or pulled anteriorly may include moving generally anteriorly. For example, the patency of upper airway 150 may increase due to the base 152 of the tongue 147 being moved and/or being permitted to move anteriorly (along at least anterior-posterior orientation) and posterior/lateral walls (at least partially defined by pharyngeal muscles) become stiffened/stretched and/or to move in an orientation (e.g., anterior-posterior, medial-lateral, and/or superior-inferior orientations), with such stiffening and/or movement acting alone (or in combination) to increase patency of the oropharynx portion 162.

The thyroid cartilage 165 is connected to pharyngeal muscles connected to the pharyngeal walls (such as oropharynx walls and/or the walls 243 illustrated by FIG. 2F) and pulling the thyroid cartilage 165 down effectively causes the pharyngeal walls (e.g., oropharynx walls) to displace and/or redistribute tissue (e.g., at least adipose tissue) in at least the oropharynx portion 162 to reduce extraluminal tissue pressure, which may increase and/or maintain patency of the at least the oropharynx portion of the upper airway 150. For example, the thyroid cartilage 165 may be connected to the inferior pharyngeal constrictor muscle, the stylopharyngeus muscle, and/or the thyrohyoid muscle.

In some examples, mechanically maneuvering the at least one of the thyroid cartilage 165 inferiorly and the hyoid bone 163 anteriorly causes a physiological effect for treating SDB that occurs remotely from the mechanically maneuvering. For example, mechanically maneuvering the thyroid cartilage 165 inferiorly occurs a distance away from the physiological effect for treating the SDB (which occurs in or near the oropharynx portion 162). The distance way may be a multiple of a diameter of the upper airway 150 of the patient. For example, and as further illustrated by FIG. 2F, the physiological effect may comprise stiffening of a pharyngeal wall(s) (e.g., at least in the oropharynx portion 162) of the patient which occurs remotely from the mechanically maneuvering action (e.g., near to reference numeral 165).

FIGS. 2B-2E are a series of diagrams schematically representing at least some different upper airway collapse patterns, including an anterior-posterior (AP) collapse pattern (FIG. 2B), a concentric collapse pattern (FIG. 2C), a lateral collapse pattern (FIG. 2D), and an anterior-posterior (AP)—lateral collapse pattern (FIG. 2E).

More particularly, FIG. 2B illustrates a diagram 210 of a collapse of the upper airway that occurs in the anterior-posterior orientation, with arrows 211 and 212 indicating an example direction in which the tissue of the upper airway collapses, resulting in the narrowed air passage 214. FIG. 2B is also illustrative of a collapse of the upper airway in the soft palate portion 160 of FIG. 2A, whether or not the collapse occurs in an anterior-posterior orientation. For example, in some instances, the velum (soft palate) portion 160 of FIG. 2A exhibits a concentric (e.g., circular) pattern of collapse, as shown in diagram 220 of FIG. 2B.

FIG. 2C illustrates a diagram 220 of a collapse of the upper airway that occurs in a concentric orientation, with arrows 222 indicating the direction in which the tissue of the upper airway collapses, resulting in the narrowed air passage 224.

FIG. 2D illustrates a diagram 230 of a collapse of the upper airway that occurs in a lateral orientation, with arrows 232 and 233 indicating the direction in which the tissue of the upper airway collapses, resulting in the narrowed air passage 235.

Various examples may include a collapse caused by a combination of orientations or patterns. For example, FIG. 2E illustrates a diagram 236 of a collapse of the upper airway that occurs in the combination of an anterior-posterior orientation (FIG. 2A) and lateral orientation (FIG. 2D), with arrows 237A, 237B, 237C indicating example directions in which the tissue of the upper airway collapses, resulting in the narrowed air passage 238. The narrowed air passage 238 may comprise a triangular shape in some examples.

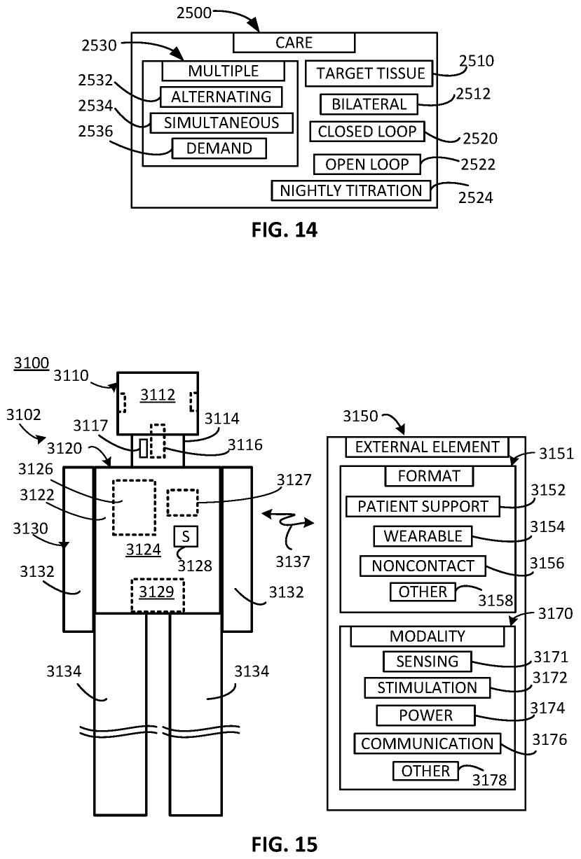

In some examples, the SDB care provided to a patient may be selected based on the exhibited collapse pattern of the upper airway. For example, the mechanically maneuvering of the thyroid cartilage and/or the hyoid bone may be selected based on the collapse pattern. In some examples, the SDB care selected may comprise a combination of mechanically maneuvering the thyroid cartilage and/or the hyoid bone, electrically stimulating at least one target tissue (e.g., upper airway patency-related tissue or other tissue), and/or using an external breathing therapy device. In some examples, patients exhibiting particular collapse patterns may be more responsive to mechanically maneuvering the thyroid cartilage, mechanically maneuvering the hyoid bone, electrically stimulating target tissue, or using an external breathing therapy device, or various combinations thereof. Further details regarding applying care to at least one target tissue is described later in association with at least FIGS. 14-15.

The responsiveness may be patient specific or general across many patients. Accordingly, the presence of or lack of a particular collapse pattern of obstruction and, optionally, a level or degree of collapse or obstruction of the upper airway, may be used to select the SDB care to provide to the patient. As some non-limiting examples, mechanical maneuvering the thyroid cartilage may result in better patient response for lateral collapse pattern as compared to anterior displacement of the anterior wall of the oropharynx through anterior displacement of the tongue.

It will be understood that various patterns of collapse occur at different levels of the upper airway portion and that the level of the upper airway in which a particular pattern of collapse appears can vary from patient-to-patient.

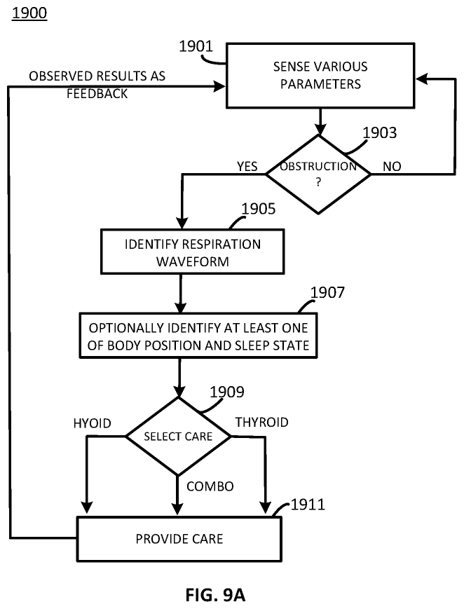

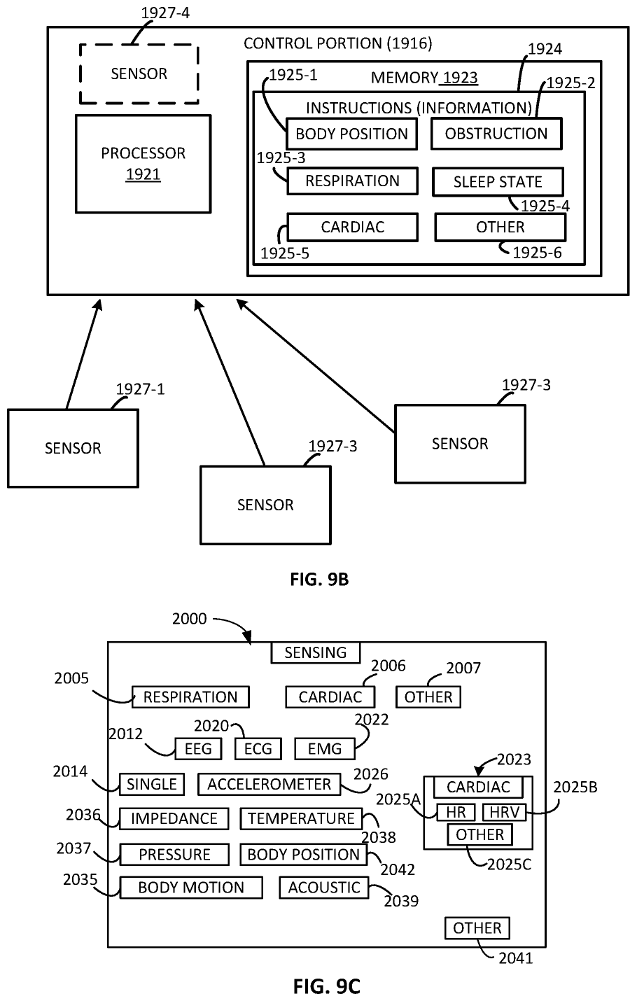

In addition to observing such collapse patterns and/or other collapse patterns, at least some aspects of such collapse patterns may be measured, such as via impedance sensing using implanted electrodes (e.g., sensing elements and/or stimulation elements), using externally applied arrays of electrodes, etc., such as further described and illustrated in association with at least FIGS. 9A-9C.

FIG. 2F are diagrams illustrating different examples of displacing adipose tissue within the upper airway responsive to the mechanical maneuvering of the at least one of the thyroid cartilage and the hyoid bone, such as in accordance with method 10 of FIG. 1. The lines in FIG. 2F illustrate walls 243 at least partially defining at least the oropharynx portion of the upper airway, which may be referred to as or include the pharyngeal walls with the base of the tongue defining the anterior portion of the oropharynx portion of the upper airway. In one aspect, the walls 243 are a surface at least partially defined by mucosal lining (skin) over adipose tissue. Accordingly, during the day and/or when the body is functioning properly during sleep, upper airway patency-related muscle (e.g., pharyngeal muscles, other muscles) constrict to displace and/or redistribute the tissue (e.g., at least adipose tissue) in at least the oropharynx portion to reduce extraluminal tissue pressure. This action may increase and/or maintain patency of the at least the oropharynx portion of the upper airway. As may be appreciated, the pharynx, including the oropharynx portion, includes a lumen 257 (e.g., hollow tube) formed by different tissue. FIG. 2F shows a simplified cross-sectional view of a portion of the oropharynx portion with respective tissue forming the lumen 257, as shown by the walls 243, and other tissue 251 defining or in the intraluminal space, such as connective tissue, adipose tissue, fat, and/or other types of tissue. The tissue forming the walls 243 may include portions of the tongue, tonsils, the soft palate, faucial pillars, the glossotonsillar sulci, uvula, pharyngeal muscles and other muscles, among other types of tissue, which form a surface (e.g., walls 243).

As described above, during sleep, at least some of the upper airway patency-related muscles may not function properly as the muscles become more relaxed, which may cause breathing obstruction as some of the tissue (at least partially forming the oropharynx portion) closes in and blocks the upper airway. The solid lines of FIG. 2F show different examples of obstruction, including pharyngeal unfolding 244, reduced wall conformity 246, and wall/tissue compression 248. In some examples, the mechanical maneuvering of the at least one of the thyroid cartilage and the hyoid bone may mimic contraction of at least one upper airway patency-related muscle, such as mimicking the contractions of the sternothyroid muscle and as shown by the dashed lines of FIG. 2F, which may be seen as pushing the walls 243 back or stiffening the walls 243 (e.g., pharyngeal walls). As such, the physiological effect caused by the mechanical maneuvering of the at least one of the thyroid cartilage and the hyoid bone may comprise stiffening of at least one pharyngeal walls (e.g., oropharynx walls) of the patient.

As used herein, upper airway patency-related muscles include and/or refer to muscles associated with increasing, restoring, or maintaining upper airway patency to promote upper airway patency. In some examples, the upper airway patency includes patency of at least the oropharynx portion of the upper airway. Some example upper airway patency-related muscles include the genioglossus muscle, which is innervated by the hypoglossal nerve. Some example upper airway patency-related muscles also may include infrahyoid strap muscles, e.g., sternohyoid, sternothyroid, thyrohyoid, and/or omohyoid muscles, which are innervated by an infrahyoid muscle (IHM)-innervating nerves. In some examples, the IHM-innervating nerves comprise those nerve branches which innervate (directly or indirectly) at least one of the respective infrahyoid strap muscles with such nerve branches being distinct from the ansa cervicalis nerve loop (e.g., including the superior root and inferior root) from which they extend. Examples are not so limited, and in some instances, upper airway patency-related muscles may comprise other muscles, innervated by other nerves.

As shown by the dashed lines of FIG. 2F, the mechanical maneuvering of at least one of the thyroid cartilage inferiorly and the hyoid bone anteriorly causes tissue (e.g., at least adipose tissue), which at least partially defines at least the oropharynx portion of the upper airway, to compress (or otherwise be manipulated) to thereby result in a dilation (e.g., an increase in a cross-sectional area 252A, 252B, 253) of at least the oropharynx portion of the upper airway. In the absence of such mechanical maneuvering, at least some of the tissue 251 (e.g., adipose) may cause portions of the wall surface of the oropharynx to protrude into (or otherwise distort, crowd, etc.) the airway passage intended for unobstructed airflow during breathing. For example, the right side/248 of FIG. 2F shows an example of a shortest cross-sectional area of the lumen 257 before the mechanical maneuvering, at 252A, and after the mechanical maneuvering, at 252B, and also a longer cross-sectional area of the lumen 257, at 253.

Accordingly, in some examples, the cross-sectional area of at least the oropharynx portion of the upper airway may increase in response to the mechanical maneuvering. The cross-sectional area may include a diameter, a shortest cross-section dimension, and/or a longest cross-sectional dimension. In this way, patency of at least the oropharynx portion of the upper airway may be increased and/or maintained by the increase in the cross-sectional area.

FIGS. 3A-3C illustrate an example implantable traction apparatus and placements for the implantable traction apparatus. The implantable traction apparatus may be used to implement the method 10 of FIG. 1, in some examples. The implantable traction apparatus may include a single device or multiple devices, such as multiple implantable winches or other types of implantable devices which are used to mechanically maneuver the thyroid cartilage inferiorly and/or the hyoid bone anteriorly. In some examples, the implantable traction apparatus may be used in addition to other devices, such as external breathing therapy device or an IMD that provides electrical stimulation of nerve(s) and/or muscle(s), as further described herein.

FIG. 3A illustrates an example of an implantable traction apparatus which comprises an implantable winch 310. The implantable winch 310 includes at least one tether 314 and at least one actuator 316 coupled to the at least one tether 314. The at least one tether 314 may be coupled to the at least one actuator 316 at a first end 309 and terminate at second end 307 that is opposite the first end 309. An anchor element 312 may be disposed on the second end 307 of the tether 314 to anchor to first non-nerve tissue. The actuator 316 or another component of the implantable winch 310 may include a second anchor element to anchor to second non-nerve tissue. Non-limiting examples of non-nerve tissue includes bone, cartilage, tendons, and/or ligaments.

In some examples, the actuator 316 includes mechanical component(s) to pull in or retract the at least one tether 314 (e.g., in direction D1), such that an effective length of the at least one tether 314 is revised (e.g., reduced from L1 to L2) and which causes the mechanical maneuvering of the thyroid cartilage or hyoid bone and displacement of tissue within the upper airway of the patient. In some examples, after pulling in or retracting the tether 314, the actuator 316 releases the at least one tether 314 or otherwise allows the at least one tether 314 to extend back out from the actuator 316 to the prior (e.g., increased) effective length (e.g., L1).

In some examples, the actuator 316 includes electrically-activated material that pulls in or retracts the at least one tether 314 or other structure (e.g., cable, passive elongate element, scissor extension arm, and which is moved in direction D1) as described above, in response to receiving an electrical signal. For example, the actuator 316 may include a gel or piezoelectric material which is has a mechanical response to an electrical signal applied thereto. The piezoelectric material could include a piezoelectric-stack or piezoelectric-bimorph. In some such examples, the actuator 316 and at least one tether 314 (or other structure) may function similar to, but may not be, a winch. In some such examples, at least one electrode may be located at, or in close proximity to, the gel or piezoelectric material and used to apply the electrical signal to the gel or piezoelectric material.

In some examples, as shown in FIG. 3A, mechanically maneuvering the at least one of thyroid cartilage and the hyoid bone comprises adjusting an effective length (e.g., retracting) of at least one tether 314 of an implantable traction apparatus, e.g., implantable winch 310, to perform at least one of pulling the thyroid cartilage inferiorly and pulling the hyoid bone anteriorly. The effective length may be adjusted from a first effective length L1 to a second shorter effective length L2. The first effective length L1 may be used when not applying therapy and/or not mechanically maneuvering the thyroid cartilage and/or the hyoid bone, sometimes herein referred to as “a non-therapy length”. The second effective length L2 may be used when applying therapy and/or to mechanically maneuver the thyroid cartilage and/or the hyoid bone, sometimes herein referred to as “a therapy length”.

In some examples, adjusting the effecting length of the at least one tether 314, such as to L2, may comprise retracting at least one tether 314 of at least one implantable winch 310 at least one of inferiorly and anteriorly via the at least one actuator 316 of the at least one implantable winch 310 that the at least one tether 314 is coupled to, such as in the direction D1 (which may correspond to arrow 325 and/or 327 of FIG. 3B). In some examples, the retraction may be temporarily or periodic, such as timed with respiration, as further described herein. In some such examples, the at least one tether 314 may be released (or otherwise the mechanical maneuvering of the thyroid cartilage and/or hyoid bone may cease or be removed), such that the at least one tether 314 returns to the prior length, L1, that may not cause or may mitigate mechanical maneuvering of the thyroid cartilage inferiorly and/or the hyoid bone anteriorly.

In some examples, tension may be applied on the at least one tether 314, such as in direction D2 that is at least somewhat opposite to direction D1. If the tension is greater than a threshold, an amount of retraction of the at least one tether 314 may be reduced and/or the at least one tether 314 may be released. For example, in response to the tension in direction D2, the effective length of the at least one tether 314 may increase from L2 and may optionally be less than L1.

In some examples, the release of the at least one tether 314 may include a complete release or may include lessening of the tension by releasing some of the line of the at least one tether 314 from the actuator 316. The release of the at least one tether 314 may be used to prevent or mitigate a component of the implantable winch 310 from being stressed beyond its intended limit. In some examples, the release of the at least one tether 314 may occur by the actuator 316 providing drag on the at least one tether 314, which causes the at least one tether 314 to be automatically pulled out in response to the tension outside the threshold. Drag on the tether 314 may include and/or refer to friction or pressure on the at least one tether 314 by a component of the actuator 316, such that the tether 314 is not pulled out from the actuator 316 until tension is on the at least one tether 314 that is greater than the friction or pressure applied by the component, similar to a fishing reel placing drag on a fishing line.

In some examples, the actuator 316 may release the at least one tether 314 in response to a signal from a sensor indicating the tension on the tether 314 is outside the threshold. For example, a parameter may be sensed, via a sensor, that is indicative of the tension on the at least one tether 314 (or an anchor element 312). In response to the parameter indicating the tension is outside the threshold, the actuator 316 may perform the at least one of reducing the amount of retraction and releasing the at least one tether 314.

In some examples, the at least one of reducing the amount of retraction and releasing the at least one tether 314 comprises causing a mechanical component coupled to the at least one tether 314 to adjust (e.g., release to some degree) in response to the tension on the at least one tether 314 being outside the threshold. The mechanical component may include a part of the actuator 316, in some examples and as described above.

While the above examples describe the release of the at least one tether 314 under some circumstances, examples are not so limited and the release may be used to adjust the length (e.g., L1 and/or L2) of the at least one tether 314 over time and/or in response to physiological events, such as coughing, swallowing, or talking by the patient, which may cause the tension on the at least one tether 314 and/or the anchor element(s) 312 to exceed a threshold.

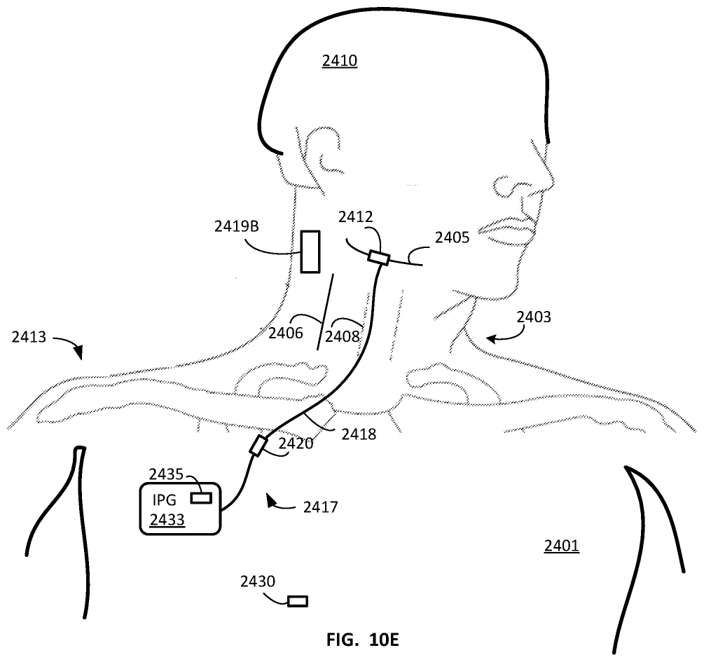

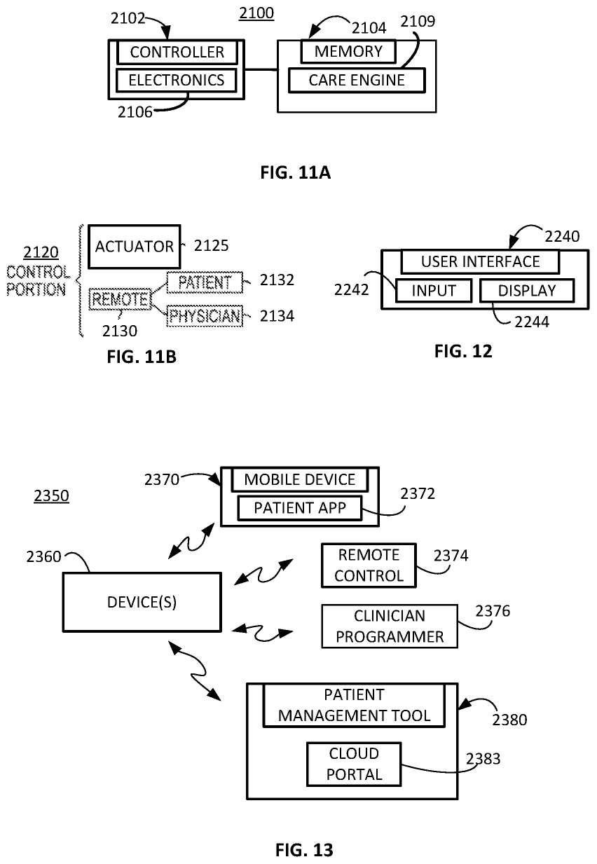

In some examples, the actuator 316 includes a processor and memory, such as a microcontroller, and a source of energy. The actuator 316 may include mechanical and/or electro-mechanical components adapted to respond to a control signal from the processor by converting the energy from the energy source into mechanical motion. Example mechanical components of the actuator 316 include a motor, rotary motor, cylinder, piston, spring, rack and pinion, gears, rails, pulleys, and/or various combinations thereof. In some examples, the processor and memory may include at least a partial implementation of, and/or at least some of substantially the same features and attributes of, the control portion 2100 and/or care engine 2109, as later described in association with at least FIGS. 11A-19. Moreover, further example implementations of the operational control aspects of the actuator are later described in association with at least FIGS. 9A-C.

As further described herein and shown by FIG. 3B, the implantable winch 310 of FIG. 3A may be implanted into a patient 320 of FIG. 3B and may operate solely mechanically, in some examples, or solely electromechanically, in some examples, to pull the thyroid cartilage inferiorly, as shown by arrow 327, and/or to pull the hyoid bone anteriorly, as shown by arrow 325. In some examples, a plurality of implantable winch devices are implanted into the patient 320 to pull the thyroid cartilage inferiorly and/or the hyoid bone anteriorly.

The particular non-nerve tissue to which the implantable winch 310 is anchored may depend on the mechanical maneuvering intended. More particularly, the at least one tether 314 of FIG. 3A may be anchored to non-nerve tissue to which upper airway-dilating muscles (e.g., that are subject to tension or pulling force) attach.

FIG. 3C illustrates different anchor locations 350 for various implantable traction apparatuses, such as the implantable winch 310 of FIG. 3A. The example anchor locations 350 include non-nerve tissue, such as a mandible 324, a hyoid bone 326, tendon 328, trachea 329, thyroid cartilage 330, sternum 332, and the clavicle 334 of a patient. In some examples, the at least one tether is anchored to one of anterior tissue and posterior tissue selected from the thyroid cartilage 330, infrahyoid muscle superior tendon 328, parts of the trachea 329, and hyoid bone 326 of the patient.

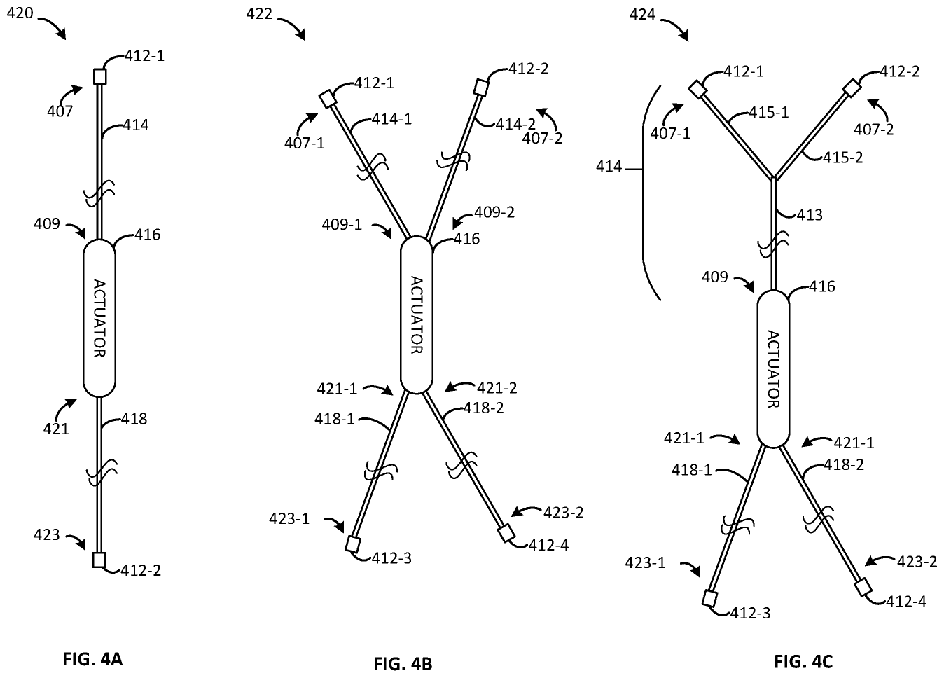

For example, if intending to mechanically maneuver (e.g., pull down) the thyroid cartilage 330 inferiorly, such as illustrated by arrow 327 of FIG. 3B, the anchor element 312 of the at least one tether 314 may be anchored to one of thyroid cartilage 330, an infrahyoid muscle superior tendon 328, and parts of the trachea 329 of the patient. Another component of the implantable winch (such as the actuator 316 as illustrated by FIG. 3A, or a second anchor element 412-2 as illustrated by FIG. 4A) may be anchored to bone structure that is inferior to the thyroid cartilage 330, infrahyoid muscle superior tendon 328, and parts of the trachea 329 of the patient to which the first anchor element is anchored to, such as to the sternum 332 or the clavicle 334. In some examples, the infrahyoid muscle superior tendon 328 may connect the thyroid cartilage 330 to muscle.

As another example, if intending to mechanically maneuver (e.g., pull forward) the hyoid bone 326 anteriorly, such as illustrated by arrow 325 of FIG. 3B, the anchor element 312 of the at least one tether 314 may be anchored to the hyoid bone 326 of the patient. Another component of the implantable winch (such as the actuator 316 as illustrated by FIG. 3A, or a second anchor element 412-2 as illustrated by FIG. 4A) may be anchored to bone structure that is anterior the hyoid bone 326, such as to the mandible 324.

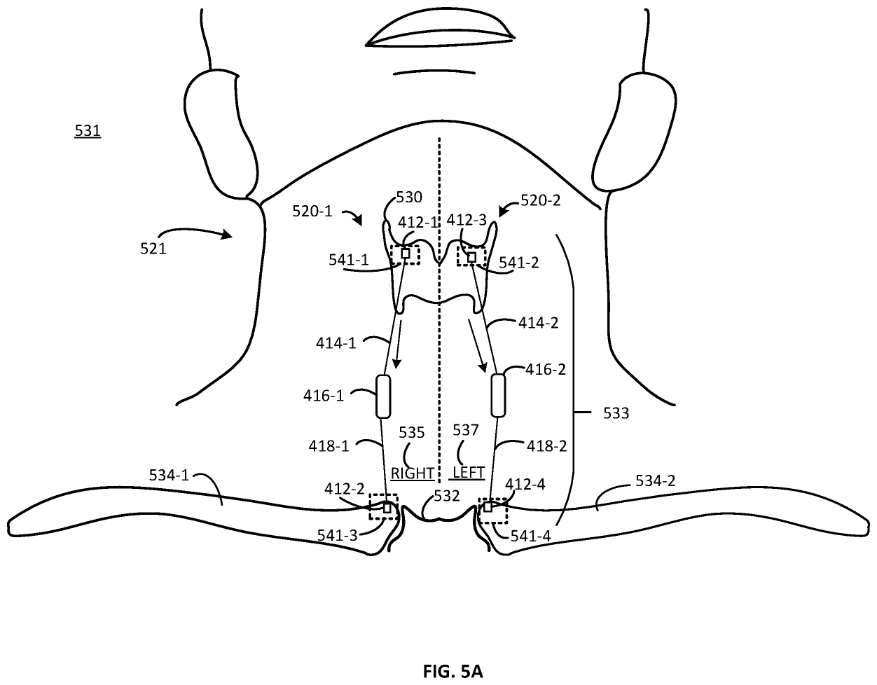

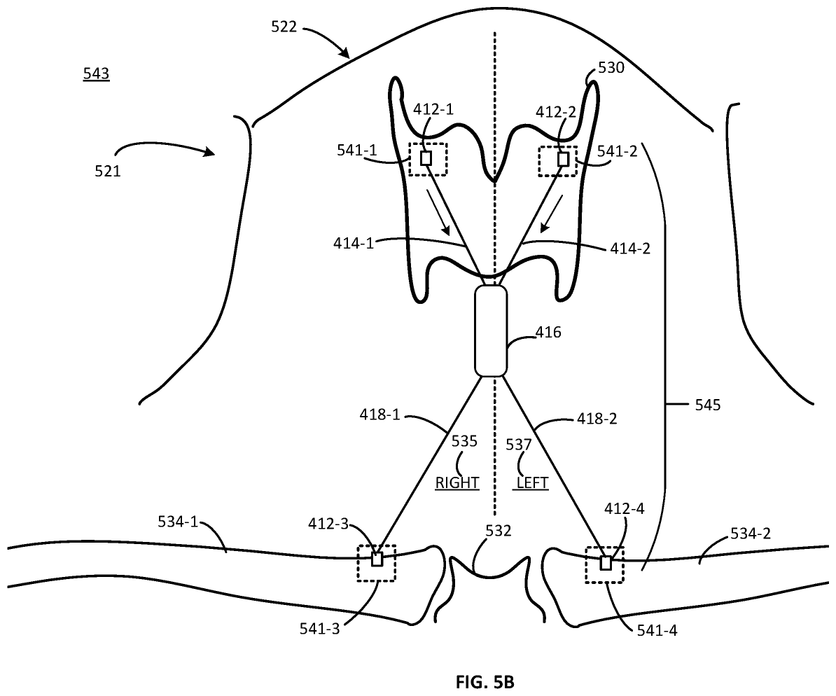

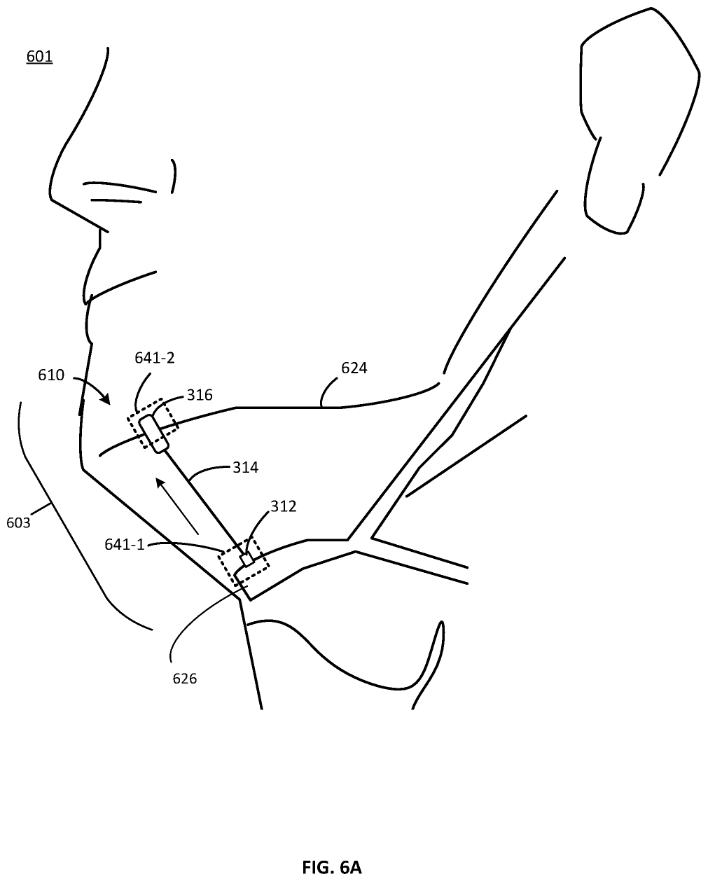

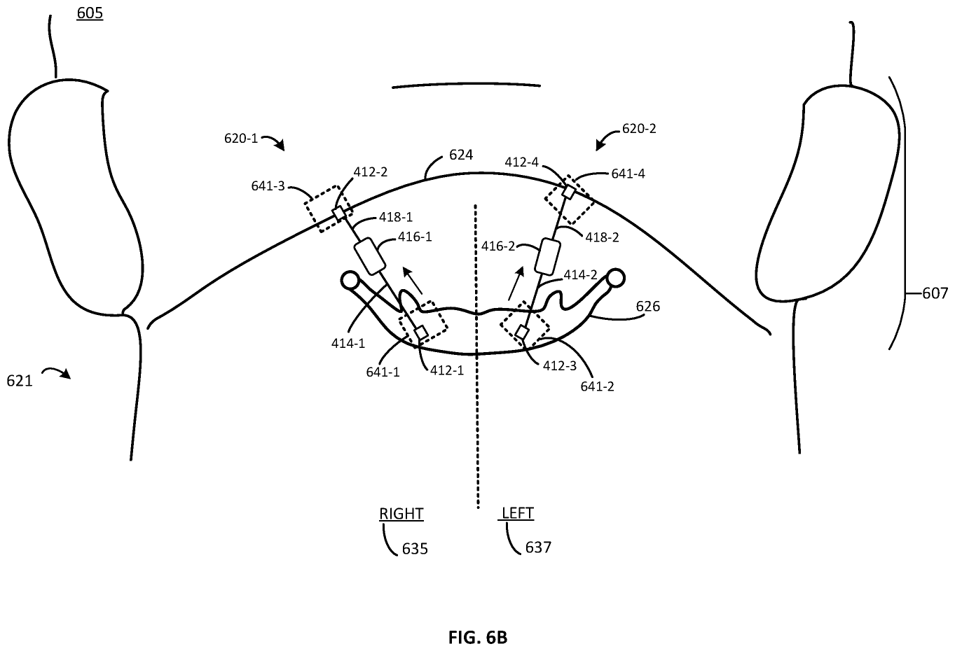

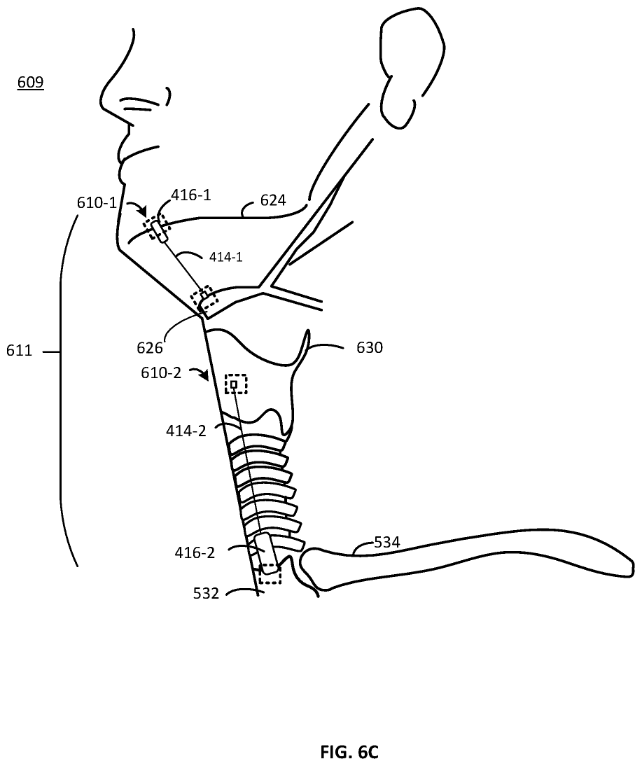

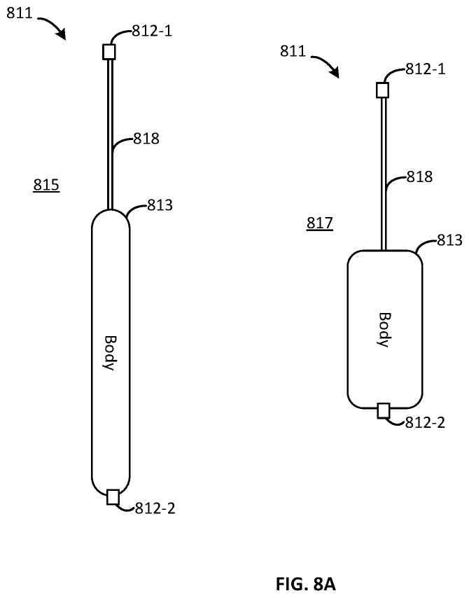

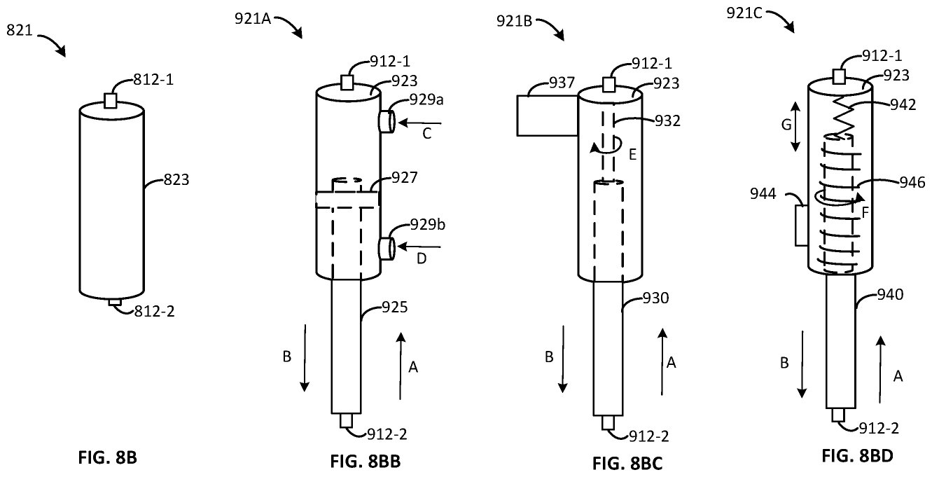

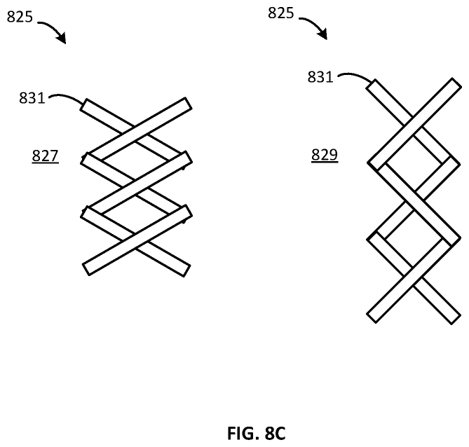

In various examples, the implantable traction apparatus may be anchored to more than two anchor locations 350. For example, the implantable traction apparatus may include two bilateral implantable winches which are respectively anchored on first and second lateral sides of the patient, such as the right side 535, 635 and left side 537, 637 shown by FIG. 5A and FIG. 6B and as viewed from the anterior. In other examples, a single implantable winch may include more than two anchor elements and may anchor on first and second lateral sides of the patient, such as shown by FIGS. 4B-4C and FIG. 5B. In further examples, multiple implantable winches may be implanted at different locations to both mechanically maneuver the thyroid cartilage 330 inferiorly and the hyoid bone 326 anteriorly, as shown by FIG. 6C. While the above examples refer to implantable winches, implantable traction apparatuses are not so limited and examples described herein include other types of devices, such as those illustrated by FIGS. 8A-8C.

FIGS. 4A-4C illustrate different example implantable traction apparatus. The implantable traction apparatuses of FIGS. 4A-4C each include implantable winches and may include an example implementation of, and/or at least some of substantially the same features and attributes as, the implantable winch 310 of FIG. 3A, with different variations as described below. For example, each of the implantable winches 420, 422, 424 of FIGS. 4A-4C include at least one tether (e.g., 414, 414-1, 414-2) coupled to at least one actuator 416. In various examples, the implantable apparatus of FIGS. 4A-4C may be used to implement any of the methods described herein, such as method 10 of FIG. 1.

As described above, and as illustrated by the implantable winch 420 of FIG. 4A, in some examples, the implantable winch 420 includes a tether 414 that includes a first end 407 to anchor to non-nerve tissue and an opposite second end 409 that is coupled to the actuator 416. The first end 407 may include a first anchor element 412-1 that may attach or be anchored to non-nerve tissue. The implantable winch 420 may further include at least one cable 418. The cable 418 includes a first end 421 coupled to the actuator 416 and an opposite second end 423 to anchor to non-nerve tissue. The second end 423 may include a second anchor element 412-2 that may attach or be anchored to non-nerve tissue.

In some examples, the tether 414 may be retracted by the actuator 416 to cause the mechanical maneuvering of the thyroid cartilage inferiorly and/or the hyoid bone anteriorly, while the cable 418 remains a constant length (e.g., is stable or not retracted). However, examples are not so limited.

In some examples, the tether 414 and cable 418 may include a continuous cable, with a mechanism in the actuator 416 that causes retraction of the first portion of the continuous cable, which functions as the tether 414 and prevents or mitigates retraction of the second portion of the continuous cable, which functions as the cable 418. For example, the at least one tether 414 may comprise: (i) a first end anchored to at least one of the thyroid cartilage, an infrahyoid muscle superior tendon, parts of the trachea, and a hyoid bone (ii) a second end opposite the first end and anchored to at least one of bone structure inferior to the thyroid cartilage (e.g., the sternum or clavicle) and bone structure anterior to the hyoid bone (e.g., mandible), and (iii) a portion between the first end and second end which is coupled to the at least one actuator 416 of the implantable traction apparatus, e.g., implantable winch 420.

In some examples, as illustrated by the implantable winch 422 of FIG. 4B, the implantable winch 422 may include multiple tethers 414-1, 414-2 and/or multiple cables 418-1, 418-2. For example, the implantable winch 422 includes a first tether 414-1 and a second tether 414-2, each with first ends 407-1, 407-2 to anchor to non-nerve tissue and opposite second ends 409-1, 409-2 that are coupled to the actuator 416. As further described herein, the first ends 407-1, 407-2 of the first and second tethers 414-1, 414-2 may be anchored to a non-nerve tissue of the patient respectively on a first lateral side and a second lateral side of the patient (e.g., left and right sides), such as via the first and second anchor elements 412-1, 412-2 on the first ends 407-1, 407-2 of the tethers 414-1, 414-2. The implantable winch 422 further includes a first cable 418-1 and a second cable 418-2. The first and second cables 418-1, 418-2 each include first ends 421-1, 421-2 coupled to the actuator 416 and opposite second ends 423-1, 423-2 to anchor to non-nerve tissue respectively on the first lateral side and the second lateral side of the patient (e.g., left and right sides), such as via the third and fourth anchor elements 412-3, 412-4 on the second ends 423-1, 423-2 of the cables 418-1, 418-2. Examples are not so limited, and in some examples, the implantable winch 422 of FIG. 4B may include a single cable or may anchor via the actuator 416.

In some examples, as illustrated by the implantable winch 424 of FIG. 4C, the implantable winch 424 may include a tether 414 with multiple ends 407-1, 407-2 to anchor to non-nerve tissue. For example, the tether 414 includes a first portion 415-1 including a first end 407-1, a second portion 415-2 including a second end 407-2, and a third portion 413 including a third end 409 that is coupled to the actuator 416. The first portion 415-1, the second portion 415-2, and the third portion 413 may join together at a junction such that the tether 414 forms a Y-shape. The first end 407-1 and second end 407-2 of the tether 414 may anchor to non-nerve tissue on a first lateral and second lateral side of the patient respectively and via first and second anchor elements 412-1, 412-2. For example, the first end 407-1 and second end 407-2 of the tether 414 may anchor to at least one of thyroid cartilage, infrahyoid muscle superior tendon, parts of the trachea, and hyoid bone respectively on the first and second lateral sides of the patient. The implantable winch 424 further includes at least one component to anchor to bone structure of the patient, such as in the middle of the bone structure or on the first lateral side and second lateral side of bone structure. The bone structure may be inferior to the thyroid cartilage or anterior to the hyoid bone. In some examples, as illustrated by FIG. 4C, the component includes at least one cable coupled to the actuator 416, such as the first and second cables 418-1, 418-2 as previously described by FIG. 4B. In other examples, the at least one component includes the actuator and, or a single cable, such as a Y-shaped cable similar to the Y-shaped tether 414.

FIGS. 5A-5B are diagrams schematically representing deployment of example implantable traction apparatus. The implantable traction apparatuses of FIGS. 5A-5B may be used to implement the method 10 of FIG. 1 and/or may include an example implementation of, and/or include at least some of substantially the same features and attributes as, any of the implantable winches illustrated by FIGS. 3A-4C. FIGS. 5A-5B illustrate deployment of implantable traction apparatuses to mechanically maneuver the thyroid cartilage 530 inferiorly.

FIG. 5A illustrates an example deployment 531 of an implantable traction apparatus 533 which comprises bilateral implantable winches 520-1, 520-1 as deployed in the neck region 521 of the patient. In some examples, each of the implantable winches 520-1, 520-2 may include an implementation of, and/or at least some of substantially the same features and attributes as, the implantable winch 420 of FIG. 4A. Examples are not so limited and any of the implantable winches illustrated by FIG. 3A and FIGS. 4B-4C may be used. For example, rather than having cables 418-1, 418-2, each of the implantable winches 520-1, 520-2 may anchor directly to the clavicle 534-1, 534-2 or sternum 532 via the actuators 416-1, 416-2 or via a multiple cables. In some example deployments, and as shown by FIGS. 5A-5B, the at least one tethers 414-1, 414-2 may be anchored to at least one of anterior or posterior thyroid cartilage 530, infrahyoid muscle superior tendon, and parts of the trachea.

As shown by FIG. 5A, the first implantable winch 520-1 may be deployed on a first lateral side of the patient, e.g., right 535, and the second implantable winch 520-2 may be deployed on a second lateral side of the patient, e.g., left 537. The anchor elements 412-1, 412-3 disposed on the first ends of the tethers 414-1, 414-2 may be anchored at anchoring locations 541-1, 541-2 associated with or comprising non-nerve tissue comprising thyroid cartilage 530, an infrahyoid muscle superior tendon (328 of FIG. 3C), or parts of the trachea of the patient (329 of FIG. 3C), with the second ends of the tethers 414-1, 414-2 being coupled to the actuators 416-1, 416-2. The anchor elements 412-2, 412-4 disposed on the second ends of the cables 418-1, 418-2 are anchored at anchoring locations 541-3, 541-4 associated with or comprising bone structure inferior to the thyroid cartilage 530 of the patient. In the example illustrated by FIG. 5A, the anchor elements 412-2, 412-4 are anchored to the clavicle 534-1, 534-2 of the patient on the first lateral side and second lateral side. Examples are not so limited and the anchor elements 412-2, 412-4 may be anchored to the sternum 532 or a combination of the clavicle 534-1, 534-2 and sternum 532.

FIG. 5B illustrates an example deployment 543 of an implantable traction apparatus 545 which comprises an implantable winch 522 as deployed in the neck region 521 of the patient. In some examples, the implantable winch 522 of FIG. 5B may include an implementation of, and/or at least some of substantially the same features and attributes as, the implantable winch 422 of FIG. 4B. Examples are not so limited and any of the implantable winches illustrated by FIG. 3A, FIG. 4A, FIG. 4C may be used. For example, rather than having cables 418-1, 418-2, the implantable winch 522 may anchor directly to the clavicle 534-1, 534-2 or sternum 532 via the actuator 416 or via a single cable.

As shown by FIG. 5B, the implantable winch 522 may be deployed on a first lateral side of the patient, e.g., right 535, via the first tether 414-1 and first cable 418-1 and on a second lateral side of the patient, e.g., left 537, via the second tether 414-2 and second cable 418-2. The anchor elements 412-1, 412-2 disposed on the first ends of the tethers 414-1, 414-2 may be anchored at anchoring locations 541-1, 541-2 associated with or comprising non-nerve tissue comprising thyroid cartilage 530, an infrahyoid muscle superior tendon (328 of FIG. 3C), or parts of the trachea of the patient (329 of FIG. 3C), with the second ends of the tethers 414-1, 414-2 being coupled to the actuator 416. The anchor elements 412-3, 412-4 disposed on the second ends of the cables 418-1, 418-2 are anchored at anchoring locations 541-3, 541-4 associated with or comprising bone structure inferior to the thyroid cartilage 530 of the patient. In the example illustrated by FIG. 5B, the anchor elements 412-3, 412-4 are anchored to the clavicle 534-1, 534-2 of the patient on the first lateral side and the second lateral side. Examples are not so limited and the anchor elements 412-3, 412-4 may be anchored to the sternum 532 or a combination of the clavicle 534-1, 534-2 and sternum 532.

With the implantable traction apparatuses 533, 545 illustrated by FIGS. 5A-5B, the actuator(s) 416, 416-1, 416-2 retract the at least one tether 414-1, 414-2 inferiorly, as illustrated by the arrows in FIGS. 5A-5B, to mechanically maneuver the thyroid cartilage 530 inferiorly. For example, the retraction of the at least one tether 414-1, 414-2 adjusts an effective length of the at least one tether 414-1, 414-2 and pulls the thyroid cartilage 530 inferiorly. The mechanical maneuvering of the thyroid cartilage 530 inferiorly may cause caudal traction of the upper airway of the patient to reduce its collapsibility, such as via the displacement of tissue (e.g., adipose) which at least partially defines the oropharyngeal walls of the upper airway and/or causes stiffening of the oropharyngeal walls. For example, at least the oropharynx of the upper airway is elongated (e.g., stretched) longitudinally via the mechanically maneuvering of the thyroid cartilage 530 inferiorly. As previously described, the mechanical maneuvering of the thyroid cartilage 530 inferiorly may mimic normal contractions of the sternothyroid muscle(s).

In various examples, the cables 418-1, 418-2 may remain stable while the at least one tether 414-1, 414-2 is retracted by the actuator(s) 416, 416-1, 416-2. For example, while retracting the at least one tether 414-1, 414-2, the at least one cable 418-1, 418-2 remains a constant length (e.g., remain stable and/or is not retracted). In some examples, having the at least one cable 418-1, 418-2 remain stable may pull the thyroid cartilage or hyoid bone rather than the actuator(s) 416, 416-1, 416-2 displacing. The actuator(s) 416, 416-1, 416-2 displacing may have a limited effect on the thyroid cartilage 530 or hyoid bone as compared to pulling thyroid cartilage 530 or hyoid bone.

FIGS. 6A-6C are diagrams schematically representing deployment of further example implantable traction apparatus. The implantable traction apparatuses of FIGS. 6A-6C may be used to implement the method 10 of FIG. 1 and/or may include an example implementation of any of the implantable winches illustrated by FIG. 3A and FIGS. 4A-4C. FIGS. 6A-6B illustrate deployment of implantable traction apparatuses to mechanically maneuver the hyoid bone 626 anteriorly and FIG. 6C illustrates deployment of an implantable traction apparatus to mechanically maneuver the thyroid cartilage 630 inferiorly and the hyoid bone 626 anteriorly.

FIG. 6A illustrates an example deployment 601 of an implantable traction apparatus 603 which comprises an implantable winch 610 as deployed in the neck region of the patient. In some examples, the implantable winch 610 of FIG. 6B may include an implementation of, and/or at least some of substantially the same features and attributes as, the implantable winch 310 of FIG. 3A. Examples are not so limited and any of the implantable winches illustrated by FIG. FIGS. 4A-4C may be used. For example, rather than the implantable winch 610 anchoring to the mandible 624 via the actuator 316, the implantable winch 610 may include at least one cable coupled to the actuator 316 and that anchors to the mandible 624, as illustrated by FIG. 6B.

As shown by FIG. 6A, the implantable winch 610 may be deployed at the center of the body of patient via the tether 314 attached at the center of the body of the hyoid bone 626 and actuator 316 attached at the center of the body of the mandible 624. At least one anchor element 312 is disposed on the first end of the tether 314 and the at least one anchor element 312 may be anchored at an anchoring location 641-1 associated with or comprising non-nerve tissue comprising the hyoid bone 626, with the opposite second end of the tether 314 being coupled to the actuator 316. The actuator 316, which may include an anchor element or is itself the anchor element, is anchored at an anchoring location 641-2 associated with or comprising bone structure anterior to the hyoid bone 626 of the patient. In the example illustrated by FIG. 6A, the actuator 316 is anchored to the mandible 624 at the center of the body of mandible 624. Examples are not so limited and the implantable winch 610 may include at least one cable coupled to and extending from the actuator 316 and which includes at least one anchor element to couple to the sides of the body of mandible 624 and/or with the least one anchor element 312 to couple to the cornu (e.g., the horns) and/or the sides of the body of hyoid bone 626.

FIG. 6B illustrates an example deployment 605 of an implantable traction apparatus 607 which comprises bilateral implantable winches 620-1, 620-2 as deployed in the neck region 621 of the patient. In some examples, each of the implantable winches 620-1, 620-2 may include an implementation of, and/or at least some of substantially the same features and attributes as, the implantable winch 420 of FIG. 3A. Examples are not so limited and any of the implantable winches illustrated by FIG. 3A and FIGS. 4B-4C may be used. For example, rather than having cables 418-1, 418-2, the implantable winches 620-1, 620-1 may anchor to the mandible 624 via the actuators 416-1, 416-2 directly.

As shown by FIG. 6B, the first implantable winch 620-1 may be deployed on a first lateral side of the patient, e.g., right 635, and the second implantable winch 620-2 may be deployed on a second lateral side of the patient, e.g., left 637. The anchor elements 412-1, 412-3 disposed on the first ends of the tethers 414-1, 414-2 may be anchored at anchoring locations 641-1, 641-2 associated with or comprising the hyoid bone 626, with the opposite second ends of the tethers 414-1, 414-2 being coupled to the actuators 416-1, 416-2. The anchor elements 412-2, 412-4 disposed on the second ends of the cables 418-1, 418-2 are anchored at anchoring locations 641-3, 641-4 associated with or comprising bone structure anterior to the hyoid bone 626 of the patient. In the example illustrated by FIG. 6B, the anchor elements 412-2, 412-4 are anchored to the mandible 624 of the patient on the first lateral side and second lateral side of the body of the mandible 624. Examples are not so limited and the anchor elements 412-2, 412-4 may be anchored to center of the body of the mandible 624 and/or anchor elements 412-1, 412-3 may be anchored to the cornu (e.g., horns) of the hyoid bone 626 and/or the center of the hyoid bone 626.

In some examples, a single implantable winch may include a first tether and a second tether to anchor to the hyoid bone 626 on the first and second lateral sides of the patient (e.g., 635, 637), such as illustrated by the implantable winches in connection with FIGS. 4B-4C. The first and the second tethers may be anchored to at least one of an anterior portion or posterior portion of the hyoid bone 626.

With the implantable traction apparatuses 603, 607 (as well as 611 of FIG. 6C, as further described below) illustrated by FIGS. 6A-6B, the actuator(s) 316, 416-1, 416-2 retract the at least one tethers 414, 414-1, 414-2 anteriorly, as illustrated by the arrows in FIGS. 6A-6B, to mechanically maneuver the hyoid bone 626 anteriorly. For example, the retraction of the at least one tether 314, 414-1, 414-2 adjusts an effective length of the at least one tether 314, 414-1, 414-2 and hyoid bone 626 anteriorly. Among other effects, the mechanical maneuvering of the hyoid bone 626 anteriorly may cause the base of the tongue (e.g., genioglossus muscle defining the anterior “wall” of the oropharynx) to be pulled anteriorly of the upper airway of the patient to increase a cross-sectional area of the oropharynx. In addition, this forced anterior movement of the hyoid bone may act to stiffen at least the lateral walls of the upper airway, which are at least partially defined by pharyngeal muscles and which may reduce their collapsibility and/or collapsibility of the posterior oropharyngeal wall. Moreover, this anteriorly directed application of tension on these pharyngeal muscles (at least partially defining the lateral walls of the oropharynx) may also cause displacement or compression of tissue (e.g., adipose, other) at least partially forming the lateral walls of the oropharynx to further promote upper airway patency. In various examples, the cables 418-1, 418-2 or the actuator 316 may remain stable while the at least one tether 414-1, 414-2 is retracted by the actuators 316, 416-1, 416-2, as previously described.

FIG. 6C illustrates an example deployment 609 of an implantable traction apparatus 611 which comprises a plurality of implantable winches 610-1, 610-2 which are disposed to mechanically maneuver both the thyroid cartilage 630 inferiorly and the hyoid bone 626 anteriorly. In some examples, the implantable winches 610-1, 610-2 of FIG. 6C may include an implementation of, and/or at least some of substantially the same features and attributes as, the implantable winch 310 of FIG. 3A.

In some examples, the plurality of implantable winches 610-1, 610-2 may include bilateral implantable winches disposed to maneuver the thyroid cartilage 630, as illustrated in connection with FIG. 5A, and/or disposed to maneuver the hyoid bone 626, as illustrated in connection with FIG. 6B. In some such examples, the implanted traction apparatus 611 may comprise more than two implantable winches, such as three, four, or more implantable winches. As a specific example, a third implantable winch comprising at least a third tether and a third actuator may be anchored (at a first end of the third tether) to at least one of thyroid cartilage 630, infrahyoid muscle superior tendon, and parts of the trachea, and coupled at a second end to a third actuator. Examples are not so limited and any of the implantable winches illustrated by FIGS. 4A-4C may be used, as previously described.

With the implantable traction apparatus 611 illustrated by FIG. 6C, the actuators 416-1, 416-2 retract at least one of the tethers 414-1 anteriorly to mechanically maneuver the hyoid bone 626 anteriorly and/or retract at least one of the tethers 414-2 inferiorly to mechanically maneuver the thyroid cartilage 630 inferiorly, as illustrated by the arrows in FIG. 6C and as previously illustrated and described in connection with FIGS. 5A-5B and 6A-6B. In some examples, the implantable traction apparatus 611 may selectively mechanically maneuver the hyoid bone 626, the thyroid cartilage 630, or both at the same time. The selective mechanically maneuvering of the hyoid bone 626 and/or the thyroid cartilage 630 may be based on at least one sensed parameter, as further described below. In some examples, both the hyoid bone 626 and the thyroid cartilage 630 may be pulled, which may have complementary effect on promoting upper airway patency.

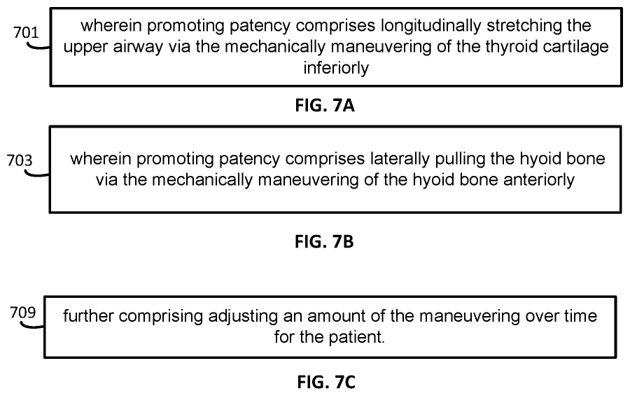

FIGS. 7A-7C illustrate example methods of promoting upper airway patency, such as patency of the oropharynx portion of the upper airway. In some examples, the methods illustrated by FIGS. 7A-B may comprise part of, and/or are example implementations of, the method 10 illustrated by FIG. 1.

As shown at 701 in FIG. 7A, in some examples, promoting patency of the upper airway comprises longitudinally elongating (e.g., stretching) the upper airway via the mechanically maneuvering of the thyroid cartilage inferiorly, such as using the deployment described and illustrated by FIGS. 5A-5B and 6C. As shown at 703 in FIG. 7B, in some examples, promoting patency of the upper airway comprises pulling the hyoid bone via the mechanically maneuvering of the hyoid bone anteriorly, such as using the deployment described and illustrated by FIGS. 6A-6C. In some examples, any of the implantable traction apparatuses illustrated by FIGS. 3A, FIGS. 4A-4C, FIGS. 5A-5B, and FIGS. 6A-6C may be used to implement the methods as illustrated by FIGS. 7A-7C. However, it will be understood that implantable traction apparatuses or other structures, devices, etc., may be used to implement the example methods of FIGS. 7A-7B.

The example methods of FIGS. 7A-7B may be further implemented via at least one (and various combinations thereof) the following example methods in FIGS. 7C, as well as FIG. 9A and the example variations described above in connection with the apparatus and devices illustrated by FIGS. 3A-6C.

As shown at 709 in FIG. 7C, in some examples, the method (e.g., method 10) may further comprise adjusting the amount of mechanically maneuvering for the patient over time. In some examples, the time period for the adjustment may include hourly, daily, weekly, monthly, yearly or more. For example, over time, the physiological features of the patient may change, such that more or less mechanically maneuvering (during select times or statically) may be used to maintain and/or increase upper airway patency. In some examples, the adjustment to the length of the at least one tether may be revised over time.

In some examples, the adjustment may include an adjustment to a first length of an implantable traction apparatus when providing treatment (e.g., to mechanically maneuver the thyroid cartilage and/or the hyoid, such as L2 of FIG. 3A) and/or an adjustment to a second length when the implantable traction apparatus is not providing treatment and/or is otherwise not mechanically maneuvering the thyroid cartilage and/or the hyoid bone (e.g., such as L1 of FIG. 3A). For example, the adjustment may include a revised first length (e.g., L2 of FIG. 3A) of the at least one tether when the implantable traction apparatus is mechanically maneuvering the thyroid cartilage and/or the hyoid bone (sometimes herein referred to as an “effective length”) and/or a revised second length (e.g., L1 of FIG. 3A) of the at least one tether when the implantable traction apparatus is not mechanically maneuvering the thyroid cartilage and/or the hyoid, such as when the patient is awake, the upper airway patency-related muscles are properly functioning, and/or at select phases of the respiration waveform (e.g., all phases other than the inspiration phase). In some instances, the patient may change anatomy over time, such over years, such that both the therapy length and non-therapy length of the at least one tether may be adjusted to accommodate the changes in anatomy.

In some examples, only the therapy length may be changed, due to changes in SDB behavior and/or responses, such as in response to feedback as further described below in connection with at least FIG. 9B.

In some examples, the effective length(s) of the at least one tether may be adjusted or revised by retracting the at least one tether to decrease the effective length or releasing line of the at least one tether to increase the effective length by the at least one actuator coupled to the opposite second end(s) of the at least one tether. In examples that include bilateral implantable winches, such as illustrated in connection with FIGS. 5A and 6B, the bilateral winches may coordinate the adjustment such that both devices are adjusted to the same revised effective length(s).

In some examples, the method of FIG. 7C may further comprise monitoring a therapy outcome such as (but not limited to) a sleep apnea rate, e.g., apnea-hypopnea index (AHI), during a treatment period, and adjusting the effective length based on the sleep apnea rate. For example, the implantable traction apparatus may adjust the effective length for providing treatment in response to the sleep apnea rate being greater than a threshold and/or increasing over time. In this manner, the implantable traction apparatus may auto-titrate.

In some examples, the adjustment to the effective length of the at least one tether (or other component of a traction apparatus) may be in response to an instruction from an external device. For example, the method of FIG. 7C may further comprise receiving an instruction to adjust the effective length. The instruction may be from a patient, such as from a remote or application, and/or from a care provider, such as from an application, as further illustrated in connection with at least FIGS. 11A-15.

Although the above describes different implantable traction apparatuses as including at least one implantable winch, examples are not so limited and may include variations, such as those further illustrated by FIGS. 8A-8C.

FIGS. 8A-8C illustrate additional example implantable traction apparatuses.

In some examples, an implantable traction apparatus and/or device may have a deformable body that changes shape in response to exposure to temperature. As shown by FIG. 8A, in some examples, the implantable traction apparatus 811 has at least one deformable body 813 formed of biodeformable material that temporarily maintains the at least one deformable body 813 in an expanded (e.g., decompressed) state, and may transform to a collapsed (e.g., normal) state which is retracted in length from the expanded state in response to exposure to temperate. FIG. 8A illustrates an implantable traction apparatus 811 with a deformable body 813 in an expanded state, at 815, and in a collapsed state, at 817.

Similar to the implantable traction devices of FIG. 3A and/or 4A, the implantable traction apparatus 811 may include at least one anchor element 812-1, 812-2 which may attach to non-nerve tissue, such as any of the anchor locations 350 illustrated in connection with FIG. 3C. By anchoring to the non-nerve tissue, the change to the collapsed state 817 may pull the thyroid cartilage inferiorly or the hyoid bone anteriorly, as described throughout the disclosure. In some examples, the anchor elements 812-1, 812-2 may include an implementation of, and/or at least some of substantially the same features and attributes as, the anchor elements described by any of FIGS. 3A-6C. In some examples, at least one of the anchor element 812-2 may be directly on the deformable body 813 and/or at least one cable 818 may extend from the deformable body 813 at a first end with at least one anchor element 812-1 at an opposite second end of the at least one cable 818. The implantable traction apparatus 811 is not limited to that illustrated by FIG. 8A and may include any of the variations as described in connection with at least FIGS. 4A-4C.

In the example of FIG. 8A, the implantable traction apparatus 811 comprises an implantable traction device. In some examples, multiple implantable traction device may form the apparatus, similar to apparatus illustrated in connection with FIG. 5A. In some examples, the implantable traction device may include multiple cables extending from each end of the deformable body 813, similar to device illustrated in connection with at least FIG. 5B.

In some examples, mechanically maneuvering the at least one of the thyroid cartilage inferiorly and the hyoid bone anteriorly may comprise attaching the implantable traction apparatus 811 to non-nerve tissue (e.g., ligament, tendon, bone) and permitting the biodeformable material of the deformable body 813 to contract, thereby causing the at least one deformable body 813 to be in a collapsed (e.g., normal) state 817 which is retracted in length from the expanded state 815 and to pull the at least one of the thyroid cartilage inferiorly and the hyoid bone anteriorly.

In some examples, permitting the biodeformable material to contract is in response to exposure to a temperature above a threshold, the threshold being associated with a body temperature of the patient. In some examples, the at least one deformable body 813 exhibits a non-linear shape when in the collapsed state 817 and exhibits a more linear shape as compared to the non-linear shape when in the expanded state 815 (e.g., straight when the apparatus 811 is deployed in the body of the patient, and then becomes non-linear in response to temperature).

In some examples, mechanically maneuvering the thyroid cartilage inferiorly and/or the hyoid bone anteriorly may comprise maintaining at least one of the thyroid cartilage in an inferior orientation and the hyoid bone in an anterior orientation via at least one of: (i) applying tension, and (ii) the at least one of thyroid cartilage and the hyoid bone being under applied tension. In this manner, the mechanical maneuvering may comprise active pulling (e.g., applying tension) and/or the application of tension from a prior step.

In some examples, the at least one of the thyroid cartilage is maintained in the interior orientation and the hypoid bone is maintained in the anterior orientation by: (i) surgically pulling at least one of the thyroid cartilage inferiorly to a first target position and the hyoid bone anteriorly to a second target position, and ii) anchoring at least one passive elongate element to non-nerve tissue to maintain at least one of the thyroid cartilage at the first target position and the hyoid bone at the second target position via the at least one passive elongate element.

FIG. 8B illustrates an example implantable traction apparatus 821 that comprises a passive elongate element 823. Example passive elongate elements include a beam, a rod, or other structure which does not have movable parts and/or is optionally not deformable.

Similar to the implantable traction devices of FIG. 3A and/or 4A, the implantable traction apparatus 821 may include at least one anchor element 812-1, 812-2 which may attach to non-nerve tissue, such as any of the anchor locations 350 illustrated in connection with FIG. 3C. The anchor elements 812-1, 812-2 may include an implementation of, and/or at least some of substantially the same features and attributes as, the anchor elements described by any of FIGS. 3A-6C. For example, at least one of the anchor element 812-1, 812-2 may be directly on the passive elongate element 823. The implantable traction apparatus 821 is not limited to that illustrated by FIG. 8A and may include any of the variations described in connection with at least FIGS. 4A-4C, such as but not limited to cable(s) extending from the passive elongate element 823.

In response to being anchored to non-nerve tissue, the position of at least one of the thyroid cartilage and the hyoid bone may be maintained via at least one passive elongate element 823. In some examples, the at least one passive elongate element 823 has a substantially fixed length. In some examples, the at least one passive elongate element 823 is semi-rigid and resilient such that a length of the at least one passive elongate element 823 changes (e.g., stretches a small percent to relieve strain on the anchor elements). In some examples, the passive elongate element 823 may change in length in a range of about 1 percent (%) to about ten %. In some examples, the passive elongate element may change lengths in a range of about 1% to about 8%, about 1% to about 5%, about 2% to about 5%, and about 5%, among other ranges.

As a non-limiting example, when implanted, the thyroid cartilage is surgically pulled inferiorly and/or the hyoid bone is surgically pulled anteriorly into the target position(s) and then anchored or sutured in the target position(s) using the passive elongate element 823 to maintain the pulled position(s). In some examples, this arrangement may be used on patients with severe crowding of upper airway, e.g., intrusion of portion of pharyngeal wall into normal lumen/area of upper airway caused by (adipose) tissue and/or other structural abnormality. In some examples, there may be a range of different lengths of passive elongate elements available and a surgeon selects an appropriate length during surgery. In some examples, the range of different lengths may be on the order of centimeters (cm), such as about 0.5 cm to about 3.0 cm, about 1.0 cm to about 3.0 cm, about 1.0 cm to about 2.5 cm, or about 1.0 cm to about 2.0 cm, among other ranges.

In the example of FIG. 8B, the implantable traction apparatus 821 comprises an implantable traction device. In some examples, multiple passive elongate elements may form the apparatus, similar to apparatus illustrated in connection with FIG. 5A. In some examples, the implantable traction device may include multiple cables extending from each end of the passive elongate element 823, similar to device illustrated in connection with FIG. 5B.

In some examples, implantable traction apparatuses may be similar to the implantable traction apparatus 821 but may be active and not passive. For example, rather than a passive elongate element 823, a body may be hollow and/or otherwise contain a movable elongate element inside that moves to change the effective length of the apparatus (e.g., a length of the elongate element extending outside the body) and to pull the thyroid cartilage inferiorly and/or the hyoid bone anteriorly. Such example devices may include linear actuators and/or linear solenoids. FIGS. 8BB-8BD show example linear actuators and linear solenoids forming implantable traction apparatuses.

FIG. 8BB illustrates an example implantable traction apparatus 921 that is a linear actuator formed of a hollow body 923 with an elongate element 925 (e.g., rod) that extends inside the body 923 and is coupled to a piston 927. The body further includes an extend port 929a and a retract port 929b. Gas or fluid may be input (via circuitry and a material source) to the extend port 929a and into the body 923 in the direction of the arrow C, which causes the elongate element 925 to move in the direction of arrow B and for the length of the elongate element 925 that extends outside the body 923 to increase. Gas or fluid may be input (via circuitry and a material source) to the retract port 929b and into the body 923 in the direction of the arrow D, which causes the elongate element 925 to move in the direction of arrow A and for the length of the elongate element 925 that extends outside the body 923 to decrease (e.g., the rod retracts). Example elongate elements include a beam, a rod, or other structure which may be movable.

Similar to the implantable traction apparatus 821, the implantable traction apparatus 921A may include at least one anchor element 912-1, 912-2 which may attach to tissue, such as any of the anchor locations 350 illustrated in connection with FIG. 3C. The anchor element(s) 912-1, 912-2 may include an implementation of, and/or at least some of substantially the same features and attributes as, the anchor elements described by any of FIGS. 3A-6C. In response to being anchored to tissue via the anchor element(s) 912-1, 912-2, the elongate element 925 may be moved in directions of arrows A and B to adjust the length of the elongate element 925 that extends outside the body 923 and to mechanically maneuver the thyroid cartilage and/or the hyoid bone.

FIG. 8BC illustrates an example implantable traction apparatus 921B that is a linear actuator formed of a hollow body 923 with a lead screw 932 coupled to an elongate element 930 that extends inside the body 923. The lead screw 932 is coupled to a gear box and motor 937 which activate to rotate the lead screw 932 in the direction of arrow E. In response, the lead screw 932 causes the elongate element 930 to move in the direction of arrow B and for the length of the elongate element 930 that extends outside the body 923 to increase. Similarly, the gear box and motor 937 may rotate the lead screw 932 in the opposite direction of arrow E, which causes the elongate element 930 to move in the direction of arrow A and for the length of the elongate element 930 that extends outside the body 923 to decrease (e.g., the rod retracts).

As with the implantable traction apparatus 921A, the implantable traction apparatus 921B may include at least one anchor element 912-1, 912-2 which may attach to tissue, such as any of the anchor locations 350 illustrated in connection with FIG. 3C. The common features are not repeated. In response to being anchored to tissue via the anchor element(s) 912-1, 912-2, the elongate element 930 may be moved in directions of arrows A and B to adjust the length of the elongate element 930 that extends outside the body 923 and mechanically maneuver the thyroid cartilage and/or the hyoid bone.

FIG. 8BD illustrates an example implantable traction apparatus 921C that is a linear solenoid formed of a hollow body 923 with an elongate element 940 (e.g., plunger) that extends inside the body 923 and is coupled to a return spring 942. As shown, coil windings (e.g., wire loops) 946 extend around the elongate element 940 and are electrically coupled to a power source 944. The power source 944 may apply electrical current to the coil windings 946 to cause the coil windings 946 to rotate in the direction of arrow F and to move the elongate element 940 in the direction of arrow A and for the length of the elongate element 940 that extends outside the body 923 to decrease (e.g., the rod retracts). Alternatively, in some examples, the power source 944 may apply electrical current to the coil windings 946 to create an electro-magnetic field along the axis of the coil, and the electro-magnetic field would result in motion of the elongate element 940. In response to the movement, the return spring 942 may compress in the upward direction of arrow G, and in response to removal of the electrical current, may release (e.g., spring) in the downward direction of arrow G and cause the elongate element 930 to move in the direction of arrow B and for the length of the elongate element 940 that extends outside the body 923 to increase.

As with the implantable traction apparatus 921A, the implantable traction apparatus 921C may include at least one anchor element 912-1, 912-2 which may attach to tissue, such as any of the anchor locations 350 illustrated in connection with FIG. 3C. The common features are not repeated. In response to being anchored to tissue via the anchor element(s) 912-1, 912-2, the elongate element 940 may be moved in directions of arrows A and B to adjust the length of the elongate element 940 that extends outside the body 923 and mechanically maneuver the thyroid cartilage and/or the hyoid bone.

Any of the implantable traction apparatuses 821, 921A, 921B, 921C of FIGS. 8A-8BD may include additional variations, such as any of those illustrated by FIGS. 3A, 4A-4C, 5A5B, and 6A-6C. For example, the implantable traction apparatuses 821, 921A, 921B, 921C of FIGS. 8A-8BD may additionally include one or more tethers which are coupled at one end to the body and at the other end to an anchor element. Additionally, examples include different variations of the linear actuators and/or linear solenoids.

Examples are not so limited, and in some examples, an implantable traction apparatus 825 may comprise a scissor extension arm 831 as shown by FIG. 8C. The scissor extension arm 831 may change lengths upon movement between a condensed state 827 and an expanded state 829. For example, the length of the scissor extension arm 831 may selectively be adjusted to pull the thyroid cartilage inferiorly and/or the hyoid bone anteriorly. The scissor extension arm 831 may be formed of a variety of materials, including but not limited to metal, polymer, and/or other material, or a combination thereof.

In some examples, the scissor extension arm 831 may comprise a plurality of struts which join together at junctions, and with each junction acting as a hinge to pivot and in response, change lengths and to transition between the condensed state 827 and the expanded state 829. In some examples, the junctions may have rounded or otherwise soft edges.

In some examples, scissor extension arm 831 may change length via a coupled actuator (not shown), and which may include an implementation of, and/or at least some of substantially the same features as any of the actuators described herein. In some examples, the implantable traction apparatus 825 may be biased with a spring and/or have tension built-in to the hinges of the scissor extension arm 831.

In some examples, the implantable traction apparatus 825 may further comprise at least one anchor element which may attach to non-nerve tissue, such as any of the anchor locations 350 illustrated in connection with FIG. 3C. The anchor elements may be attached directly to the ends of the scissor extension arm 831 and/or cables or other components may be attached to the ends of the scissor extension arm 831, with the anchor element coupled to the cables or other components, as previously described. The anchor elements may include an implementation of, and/or at least some of substantially the same features and attributes as, the anchor elements described by any of FIGS. 3A-6C.

In some examples, the implantable traction apparatus 825 comprises an implantable traction device, such as with the ends of the scissor extension arm 831 coupling to non-nerve tissue on a left and right side of a patient. In some examples, the anchor elements may couple to the center of a non-nerve tissue. In some examples, the implantable traction apparatus 825 may include multiple scissor extension arms.

FIGS. 9A-9C are diagrams schematically representing an example method, control portion, sensors, and sensing portion for selectively applying different care based on at least one sensed parameter and sensing of the at least one parameter. The diagrams may comprise part of, are example implementations of, and/or may be used to implement method 10 of FIG. 1 and/or any of the variations as described through the present disclosure and in connection with FIGS. 2A-8C. Furthermore, any of the apparatuses and/or devices illustrated and described in connection with FIGS. 2A-8C may be used to implement the method illustrated by FIG. 9A, may include the control portion of FIG. 9B, and/or may include the sensing portion of FIG. 9C.

FIG. 9A is a flow diagram illustrating an example method 1900 for selectively applying care based on at least one sensed parameter sensed via at least one sensor. In some examples, method 1900 may comprise an example implementation of, and/or be performed via at least some of substantially the same features and attributes as, the example devices, assemblies, circuitry, managers, modules, engines, functions, parameters, respiration determination elements, stimulation elements, actuators pulse generators, sensors, electrodes, and/or methods described in association with FIGS. 1-8C and 9B. In some examples, method 1900 may be performed via at least some devices, assemblies, circuitry, managers, modules, engines, functions, parameters, respiration determination elements, stimulation elements, actuators, pulse generators, sensors, electrodes, and/or methods other than those described in association with FIGS. 1-8C and 9B-9C.

In some examples, the method 1900 of applying care is optionally based on at least one parameter sensed via at least one sensor. As further illustrated by FIG. 9B, the at least one sensor may be integrated with (or form part of) the control portion 1916 (e.g., a portion of an actuator) and/or may be external to the control portion 1916, such as an implanted sensor, a wearable sensor, or other types of sensors external to the patient, external therapy components, etc., at least some aspects of which are further described later in association with example arrangement 3100 of FIG. 15.

In some examples, the method 1900 of FIG. 9A may be performed by a control portion as illustrated by 2100 of FIG. 11A, the care engine 2500 as illustrated by FIG. 14 and/or using control signals based on information communicated from sensors (e.g., wireless or wired). The control signals may be based on sensor signals indicative of the parameters and which cause selective mechanical maneuvering the thyroid cartilage inferiorly and/or the hyoid bone anteriorly, as described in connection with at least the method 10 of FIG. 1. In some examples, the control portion 1916 may form part of an actuator of an implantable traction apparatus. In some examples, the control portion 1916 may be separate from, but in communication with, the actuator of an implantable traction apparatus.

In some examples, the control portion 1916 comprises a memory 1923 which may store machine readable instructions (and/or store information) 1924 executable on processor 1921. Among other instructions (and/or stored information), in some examples the instructions (and/or stored information) 1924 may comprise a body position parameter 1925-1, an obstruction parameter 1925-2, respiration parameter 1925-3, sleep state parameter 1925-4, cardiac parameter 1925-5, and/or other parameter 1925-6. Among other uses, these parameters (alone or in various combinations) may provide or correspond to physiologic signals (and/or information derived therefrom) by which patient care may be provided such as (but not limited to) sensing, delivering therapy, tracking, evaluation, etc. according to various examples of the present disclosure.

In some examples, as described above, mechanically maneuvering of the thyroid cartilage and/or hyoid bone is based on or in response to the at least one parameter sensed via sensor. In such examples, the mechanically maneuvering of the thyroid cartilage and/or hypoid bone may be temporary and/or periodic. For example, in response to the sensed parameter, the mechanical maneuvering of the thyroid cartilage and/or hyoid bone is selectively caused to occur. The sensed parameter may be indicative of at least one of: (i) respiration of the patient, (ii) a body position of the patient, (iii) a sleep state of the patient, and (iv) upper airway obstruction of the patient.

In some examples, the at least one sensed parameter is associated with respiration of the patient, e.g., respiration parameter 1925-3. For example, the mechanically maneuvering of the at least one of the thyroid cartilage inferiorly and the hyoid bone anteriorly may be timed with (e.g., relative to) a fiducial of a respiration waveform (e.g., cycle) of the patient. In some examples, timing the mechanically maneuvering with a fiducial(s) of the respiration waveform may include triggering and/or synchronizing the mechanical movement with the fiducial(s) of the respiration waveform. For example, mechanically maneuvering the at least one of the thyroid cartilage inferiorly and the hyoid bone anteriorly is synchronized with an inspiration phase of the respiration waveform (e.g., to mimic contractions of the sternothyroid muscle). At other phases of the respiration waveform, the thyroid cartilage and hyoid bone may not be mechanically maneuvered, such that thyroid cartilage and/or hyoid bone is allowed to return to a normal or relaxed orientation and/or the mechanically maneuvering is reduced.

In some examples, a parameter indicative of respiration of the patient may be sensed via the at least one sensor and a respiration waveform may be identified from the parameter 1925-3. In some examples, the parameter indicative of respiration may be sensed via a sensor which is internal to the actuator or external to the actuator of the implantable traction apparatus, and in communication therewith. The identification of the respiration waveform may be performed by a processor of the actuator, and may be stored on memory therewith, or another control portion, as further described in connection with at least FIGS. 9B-9C and 11A-15.

In various examples, multiple parameters may be used. The method 1900 of FIG. 9A illustrates an example of using multiple parameters to time the mechanical maneuvering of the thyroid cartilage and/or hyoid bone. For example, the method 1900 may comprise selectively causing the mechanical maneuvering of the at least one of the thyroid cartilage inferiorly and the hyoid bone anteriorly based on at least one of the sleep state (parameter 1925-4), respiration information (parameter 1925-3), the body position (parameter 1925-1), cardiac information (parameter 1925-5), other sensed physiologic signals/information (parameter 1925-6), temporal information (e.g., a time of day), and/or selectively stopping or not providing the mechanical maneuvering of the at least one of the thyroid cartilage inferiorly and the hyoid bone anteriorly.

As shown at 1901 in FIG. 9A, the method 1900 comprises sensing the various parameters. As shown by FIG. 9B, the different parameters may be indicative of body position (parameter 1925-1), upper airway obstruction (parameter 1925-2), respiration information (parameter 1925-3, e.g., waveform morphology, respiratory cycle, phase information, rate, rate variability, etc.), sleep state (parameter 1925-4), cardiac information (parameter 1925-5, e.g., waveform morphology, heart rate, heart rate variability, etc.) and/or other (parameter 1925-6). Different sensors may sense and communicate the sensor signals to the control portion (e.g., actuator) to determine the parameters from the sensor signals.

In response to the various parameters, as shown at 1903 in FIG. 9A, the method 1900 comprises determining if there is an upper airway obstruction detected. If no upper airway obstruction is detected, the method 1900 includes continuing to collect sensed parameters. In response to an upper airway obstruction, as shown at 1905 in FIG. 9A, the method 1900 comprising identifying the respiration waveform of the patient (which may be performed at a different time, such as prior to that illustrated by FIG. 9A), and optionally as shown at 1907, identifying at least one of a body position and sleep state of the patient. Based on the respiration waveform, and optionally the body position and/or sleep state of the patient, as shown at 1909 in FIG. 9A, the method 1900 comprises selecting SDB care for the patient. In some examples, at 1905/1907/1909 sensed physiologic information in addition to, or instead of, the respiratory waveform (parameter 1925-3), body position (parameter 1925-1), and/or sleep state (parameter 1925-4) or other sleep information may be used to select SDB care for the patient. At least some other/additional physiologic information, as well as the respiratory information, body position, and/or sleep information, is further described in association with at least FIGS. 9A-9C, 10A-10E, and 11A-15.

It will be understood that some examples may comprise providing SDB care such as, but not limited to, the aforementioned mechanical maneuvering without first detecting obstructions. In other words, in order to prevent or minimize SDB, some example methods may perform SDB case without waiting to see whether or when OSA events begin occurring, such as initiating therapy at the beginning of a nightly treatment period without first observing OSA events. Further, while some examples refer to treatment in response to an SDB event, the treatment may be response to an SBD event rate being above a threshold.

In some examples, the selection of SDB care may include selectively pulling the thyroid cartilage inferiorly, pulling the hyoid bone anteriorly, and/or a combination care of both pulling the thyroid cartilage inferiorly and pulling the hyoid bone anteriorly. In some examples, the selected SDB care may comprise one (or both) of the pulling the thyroid cartilage inferiorly and the hyoid bone anteriorly and an additional care, such as providing electrical stimulation to a nerve or muscle and/or activation of an external breathing therapy device.

In response to the selection, the care is provided, as shown at 1911 in FIG. 9A. For example, SDB care may be provided in response to identifying the patient is in a sleep state and has an upper airway obstruction. In some examples, the SDB care provided may include mechanically maneuvering the thyroid cartilage inferiorly and/or the hyoid bone anteriorly and as timed with the inspiration phase (and/or other fiducial, phase information, etc.) of the respiration waveform. In some examples, by timing with the inspiration phase of the respiration waveform, the mechanical maneuvering may mimic upper airway patency-related muscles to maintain or increase upper airway patency. At other phases of the respiration waveform, the thyroid cartilage and/or hyoid bone may be allowed to return to a relaxed or normal orientation, and/or otherwise are not mechanically maneuvered or the mechanically maneuvering is reduced. For example, with an implantable winch, the actuator may release the at least one tether or otherwise allow the tether to extend in length, such that the at least one tether may return to normal or non-treatment length (e.g., L1 of FIG. 3A).

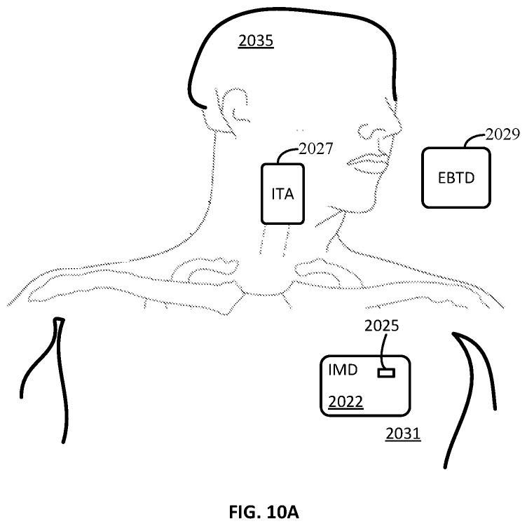

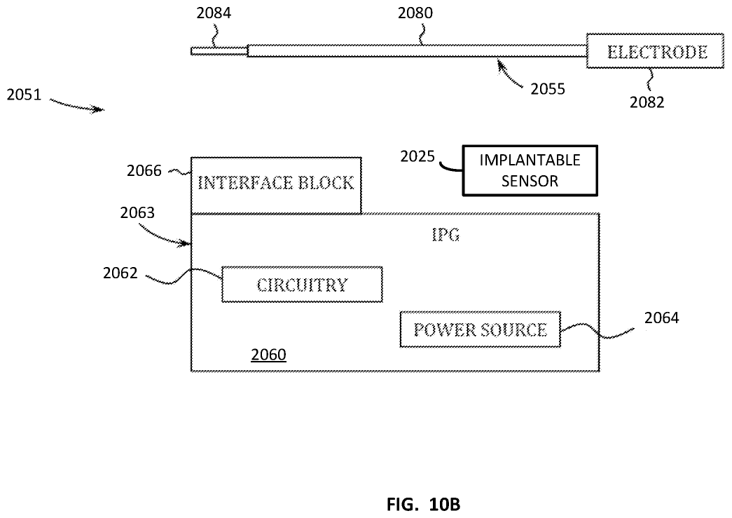

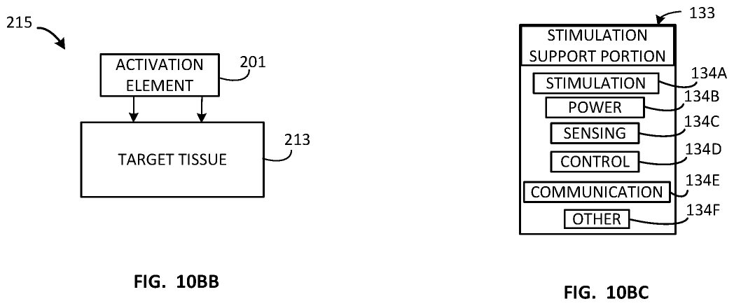

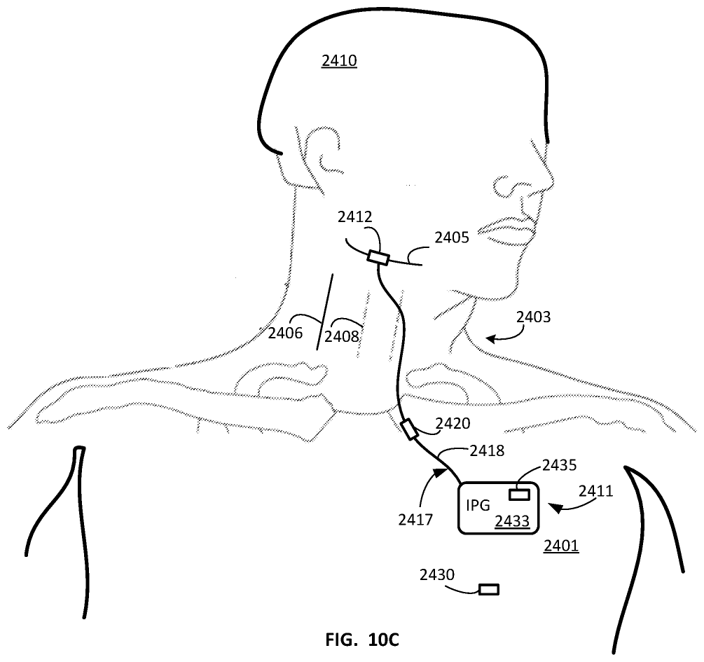

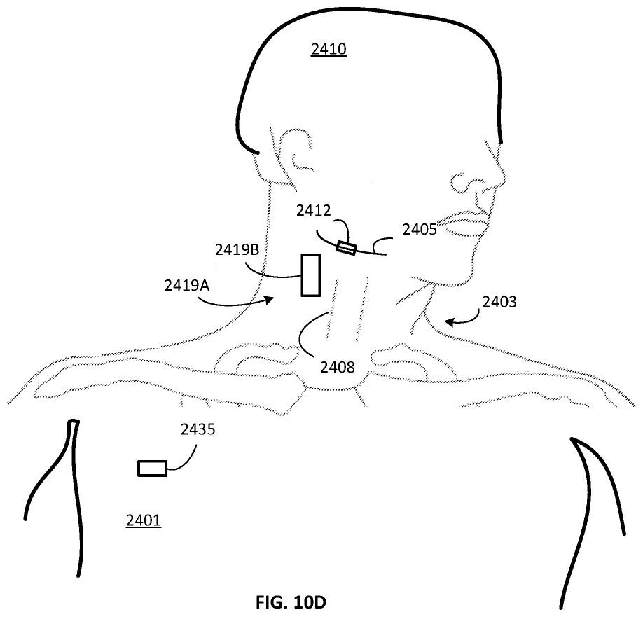

In some examples, another device may be used in addition to the implantable traction apparatus, with the SDB care being selectively provided based on the at least one sensed parameter. Such device may include a medical device (MD) which is used to stimulate nerves and/or muscles related to the upper airway patency, whether components of the MD are implantable and/or external to the patient. At least some example MDs are illustrated by FIGS. 10A-10E and FIG. 15.

As previously noted in relation to the method 1900 of FIG. 9A, FIG. 9B illustrates an example control portion 1916, which may be used to implement the method 1900 of FIG. 9A and/or any of the methods described herein. The control portion 1916 of FIG. 9B may include an implementation of, and/or at least some of substantially the same features and attributes as, the actuators 316, 416 illustrated by any of FIGS. 3A, 4A-4C, 5A-5B, and 6A-6C, the control portion as further described by any of FIGS. 11A-13, the care portion as further described by FIG. 14, and/or the stimulation portion as further described by FIG. 15

As shown by FIG. 9B, the control portion 1916 includes processor 1921 and memory 1923 that stores machine readable instructions, as further described in connection with FIG. 11A. The processor 1921 may execute the machine readable instructions stored on the memory 1923, which when executed cause the processor 1921 to perform the above-identified actions, including but not limited to the method 10 as described in connection with FIG. 1, any of the methods of FIGS. 7A-7C, method 1900 (FIG. 9A), and/or other examples throughout the present disclosure, and thereby causing the mechanical maneuvering of the thyroid cartilage inferiorly and/or the hyoid bone anteriorly, electrical stimulation therapy, and/or external breathing therapy. In some examples, control portion 1916 may form part of the actuator of an implantable traction apparatus. In some examples, control portion 1916 may form part of another device (e.g., pulse generator (PG) or implantable pulse generator (IPG)) which is in communication with the actuator of an implantable traction apparatus, and with the actuator including a separate processor, memory, and optionally a power source. In some examples, the control portion 1916 and/or actuator of the implantable traction apparatus may receive power from an external power source, such as via an external element 3150 as further described in connection with FIG. 15. For example and as further described herein, the control portion 1916 may provide instructions to the actuator and/or an electrode at or near the actuator (e.g., gel or piezoelectric material) to cause mechanical maneuvering of the thyroid cartilage and/or hyoid bone.

The control portion 1916 may be in communication with the sensors 1927-1, 1927-2, 1927-3, 1927-4 via a wired or wireless communication link, among other components. In response to received sensor signals, the processor 1921 may identify various parameters, such as the above-mentioned parameters regarding body position (parameter 1925-1), upper airway obstruction (parameter 1925-2), respiration information (parameter 1925-3), sleep state (parameter 1925-4), cardiac information (parameter 1925-5), and/or other information (parameter 1925-6), and may store the parameters on memory 1923. The processor 1921 may use the stored parameters to determine SDB care to provide to the patient at a particular date and time. In some examples, the identified parameters (e.g., other 1925-6) may further comprise a displacement parameter which is indicative of a position of at the thyroid cartilage and/or hyoid bone as sensed via a displacement sensor, as further described below.

The sensors 1927-1, 1927-2, 1927-3, 1927-4 may include a variety of different types of sensors. Example sensors include an acceleration sensor, a pressure sensor, an impedance sensor, an airflow sensor, a radio frequency sensor, electromyography (EMG) sensor, electrocardiography (ECG) sensor, ultrasonic, acoustic sensor, image sensor, displacement sensor, and/or other types of sensors. Each of the sensors may be implemented as an external sensor and/or an implantable sensor.

It will be understood that any of the parameters (1925-1, 1925-2, 1925-3, 1925-4, 1925-5, 1925-6) may be determined, tracked, etc. via one or more of the different sensor modalities (alone or in combination) as described in association with sensors 1927-2, 1927-3, 1927-4, with some more specific examples further described below.

An acceleration sensor may include accelerometer (e.g., a multi-axis accelerometer such as a three-axis or six-axis accelerometer), a gyroscope, etc., and may be used to identify the body position (parameter 1925-1) and/or sleep state (parameter 1925-4) of the patient.

The acceleration sensor may sense an amount of acceleration, which may be used to identify body motion and posture, e.g., body position (parameter 1925-1). Example body motions include movement in a vector or a direction (e.g., walking, running, biking), rotational motions (e.g., twisting), and changes in posture (e.g., change from an upright position to a sitting or supine position), among other movements. The motion may be sensed relative to a gravity vector, such as an earth gravity vector and/or a vertical baseline gravity vector for calibrating the data. In various examples, the sensed force(s) may be processed to determine a posture of the patient. As used herein, posture includes and/or refers to a position or bearing of the body. The term “posture” may sometimes be referred to as “body position”. Example postures include upright or standing position, supine position (e.g., generally horizontal body position), a generally supine reclined position, sitting position, etc.

In some examples, the acceleration sensor may be used to sense additional physiological data. The additional physiological data may include additional physiological parameters, such as (but not limited to) cardiac signals/information per parameter 1925-5 and/or respiration signals/information per parameter 1925-3. As further described herein, the respiration information may be determined based on rotational movements of a portion of a chest wall of the patient during breathing. For example, the acceleration sensor may be used to determine respiration information, cardiac information, detection of SDB events, sleep information, and/or other information or be implemented according to at least some of substantially the same features and attributes as described within: U.S. Pat. No. 11,324,950, granted on May 5, 2033, entitled “ACCELEROMETER-BASED SENSING FOR SLEEP DISORDER BREATHING (SDB) CARE”; U.S. Patent Publication No. US2023/0119173, published on Apr. 20, 2023, and entitled “RESPIRATION DETECTION”; U.S. Patent Publication No. US2023/0277121, published on Sep. 7, 2023, and entitled “DISEASE BURDEN INDICATION”; and PCT Publication No. WO2022/261311, published on Dec. 15, 2022, and entitled “RESPIRATION SENSING”, the entire teachings of which are each incorporated herein by reference in their entirety.

In some examples, a pressure sensor may sense pressure, sound, and/or pressure waves. The pressure, sound, and/or pressure ways may be indicative of and/or used to determine different parameters such as respiration parameters (e.g., respiration waveform, inspiration, exhalation, rate, etc.), heart rate, electrocardiogram information (e.g., QRS complex, heart rate variability), SDB events, among other parameters. For example, one pressure sensor may comprise an implantable respiratory sensor. In some examples, pressure sensor comprises piezoelectric element(s). Although examples are not so limited.

In some examples, an airflow sensor may be used to sense respiration information (parameter 1925-3), upper airway obstruction (parameter 1925-2) or other SDB related information, sleep quality information, etc. In some instances, the airflow sensor detects a rate or volume of upper respiratory airflow.

In some examples, an impedance sensor may sense a bio-impedance signal. The bio-impedance signals may be indicative of and/or used to determine parameters, such as an upper airway obstruction/parameter 1925-2 or SDB events, cardiac information/parameter 1925-5 (e.g., ECG signal, derived cardiac metrics, etc.), respiration information/parameter 1925-3 (e.g., respiratory cycle, etc.), including or indicative of the inspiratory and/or expiratory phases and inspiratory rate, among other parameters. The impedance sensor may be implemented as various sensors distributed about the upper body, whether the sensors are internal and/or external to the patient.

In some examples, a radio frequency (RF) sensor is used to enable non-contact sensing of various additional physiologic parameters and information, such as but not limited to respiration information (parameter 1925-3), cardiac information (parameter 1925-5), motion/activity, and/or sleep information (parameter 1925-4, e.g., sleep quality, other). In some examples, RF sensor determines chest motion based on Doppler principles, which may be used to sense respiration information (parameter 1925-3), cardiac information (parameter 1925-5), etc. The RF sensor may be embodied as the electromagnetic field sensor, in some examples.

In some examples, the at least one sensor includes an optical sensor. The optical sensor may sense heart rate and/or oxygen saturation via pulse oximetry, and/or oxygen desaturation index (ODI).

An EMG sensor may be used to record and evaluate electrical activity produced by muscles, whether the muscles are activated electrically or neurologically. In some instances, the EMG sensor is used to sense respiration information (parameter 1925-3), such as but not limited to, respiration rate and phase information, and/or upper airway obstruction (parameter 1925-2).

In some examples, an ECG sensor may be used which produces an ECG signal. In some instances, the ECG sensor comprises a plurality of electrodes distributable about a chest region of the patient and from which the ECG signal is obtainable. In some examples, ECG sensor is used to detect upper airway obstruction (parameter 1925-2), respiration information (parameter 1925-3), and/or cardiac information (parameter 1925-5).

In some examples, an ultrasonic sensor may be used to detect an ultrasonic signal. In some instances, ultrasonic sensor is locatable in close proximity to an opening (e.g., nose, mouth) of the patient's upper airway and via ultrasonic signal detection and processing, may sense exhaled air to enable determining at least respiration information (parameter 1925-3), sleep quality information, upper airway information, such as upper airway obstruction (parameter 1925-2), etc.

In some examples, an acoustic sensor comprises piezoelectric element(s), accelerometers, etc., which sense acoustic vibration. In some instances, such acoustic vibratory sensing may be used to detect snoring which may be indicative of at least, upper airway obstruction (parameter 1925-2), respiration information (parameter 1925-3 and including SDB information in addition to obstruction).

In some examples, as tracked via other parameter 1925-6, a displacement sensor may be used to produce a sensor signal indicative of a position of the thyroid cartilage and/or hyoid bone. In some examples, the sensor signal from the displacement sensor may be indicative of an amount of displacement or mechanical maneuvering of the thyroid cartilage and/or hyoid bone, which may be used as feedback. For example, the control portion 1916 may assess the amount of displacement with an amount and/or increase patency of the upper airway. Such feedback may be used to determine how much and/or when to apply the mechanical maneuvering of the thyroid cartilage and/or hyoid bone, which may vary from patient to patient and/or for a patient over time. As a specific example, certain patients may benefit from pulling the hyoid bone anteriorly rather than pulling the thyroid cartilage inferiorly, or vice versa. As another example, the feedback may be used to adjust the amount of mechanically maneuvering for the patient over time, such as previously described in connection with FIG. 7C. In some examples, the sensor signal from the displacement sensor may be indicative of the thyroid cartilage and/or hyoid bone being in a non-therapy position, such as indicating to mechanically maneuver the thyroid cartilage and/or hyoid bone. The displacement sensor may be an impedance sensor, an accelerometer or other types of sensors that are placed on and/or near the thyroid cartilage and/or hyoid bone to sense a position associated therewith.

FIG. 9C is a block diagram schematically representing an example sensing portion of an example device and which may be used to implement the method 900 of FIG. 9A. In some examples, an example method may employ an example SDB care device (e.g., including an implantable winch device or other devices illustrated by FIGS. 3A-8C and/or a stimulation element 201 illustrated by FIGS. 2B-2C) comprising the sensing portion 2000 to sense physiologic information and/or other information, with such sensed information relating to care of a wide variety of physical conditions such as, but not limited to, SDB care, pelvic care, cardiac care, among other uses. In some examples, the sensing portion 2000 of FIG. 9C may be implemented by an actuator, a pulse generator (PG), or control portions described and/or illustrated by any of FIGS. 3A-14 and/or may include an implementation of, and/or at least some of substantially the same features and attributes as control portion 2100 of FIG. 11A. In some examples, the sensing portion 2000 may be implemented by a component/device external to the PG and/or SDB care device such that the control portion 1916 may form part of a different device than sensing portion 2000, such as illustrated in connection with FIG. 15.

The sensed information may be used to implement at least some of the example methods and/or examples devices described in association with at least FIGS. 1-8C. For example, the thyroid cartilage and/or the hyoid bone may be mechanically maneuvered in accordance with the method of FIG. 1 and/or using any of the devices of FIGS. 3A-6C and/or FIGS. 8A-8C and, optionally, as timed with or using the sensed information (e.g., timed with the fiducial(s) of the respiration waveform). It will be understood that the sensing portion 2000 may be implemented as single sensor or multiple sensors, and may comprise a single type of sensor or multiple types of sensing. In addition, it will be further understood that the various types of sensing schematically represented in FIG. 9C may correspond to a sensor and/or a sensing modality.

In some examples, the sensed information may refer to physiologic signals (e.g., biosignals) and/or metrics which may derived from such physiologic signals. For example, among other sensed physiologic signals, one physiologic signal may comprise respiration (parameter 2005 in FIG. 9C), from which various metrics may be derived such as, but not limited to, respiratory rate, respiratory rate variability, respiratory phase, rate times volume, waveform morphology, and more. The respiration information may be sensed via at least one of the sensing modalities described below (and/or other sensing modalities) such as, but not limited to, accelerometer 2026, ECG 2020, impedance 2036, pressure 2037, temperature 2038, acoustic 2039, and/or other sensing modalities, at least some of which are further described below. The respiration information may be used for a wide variety of purposes such as, but not limited to, timing the mechanically maneuvering and/or stimulation of tissue relative to respiration, disease burden, sleep-wake status, arousals, etc. In some such examples, the detection of disease burden may comprise detection of SDB events, which may be used in determining, assessing, etc. therapy outcomes such as, but not limited to, AHI.

In some examples, the sensed physiologic information may comprise cardiac information (2006) obtained from a cardiac signal and from which various metrics may be derived such as, but not limited to, heart rate (HR), heart rate variability (HRV), P-R intervals, waveform morphology, and more. One example of a cardiac signal may comprise an ECG signal, as represented at 2020 in FIG. 9C. Accordingly, the cardiac information and/or signal may be sensed via at least one sensing modality further described below (and/or other sensing modalities) such as, but not limited to, cardiac sensor 2023, accelerometer 2026, ECG 2020, EMG 2022, impedance 2036, pressure 2037, temperature 2038, and/or acoustic 2039. In some examples, the sensed physiologic information (e.g., via sensing portion 2000) may comprise a wide variety of physiologic information other (2007) than respiration and/or cardiac information, with at least some examples.

The sensed physiologic signals and/or information (e.g., respiration 2005, cardiac 2006, and/or other information 2007) may be used for a wide variety of purposes such as, but not limited to, determining sleep-wake status (e.g., various sleep onset determinations), timing stimulation relative to respiration, determining disease burden, determining arousals, etc. In some such examples, the determination of disease burden may comprise detection of SDB events, which may be used in determining, assessing, etc. therapy outcomes such as, but not limited to, AHI, as well as titrating stimulation parameters, adjusting sensitivity of sensing the physiologic information, etc.

For instance, in one non-limiting example, an ECG sensor 2020 in FIG. 9C may comprise a sensing element (e.g., electrode) or multiple sensing elements arranged relative to a patient's body (e.g., implanted in the transthoracic region) to obtain ECG information. In some examples, the ECG information may comprise one example implementation to obtain cardiac information, including but not limited to, HR 2025A, HRV 2025B, and other cardiac parameters 2025C, which may be used (with or without other information) in determining delivering therapy (e.g., mechanically maneuvering and/or stimulation therapy) and associated sensing (e.g., inputs) for determining effectiveness of the therapy and/or implementing the therapy, as described throughout the examples of the present disclosure.

However, in some instances, the ECG sensor 2020 may represent ECG sensing element(s) in general terms without regard to a particular manner in which sensing ECG information may be implemented.

In some examples in which multiple electrodes are employed to obtain an ECG signal, an ECG electrode may be mounted on or form at least part of a case (e.g., outer housing) of a stimulation support portion (which may comprise an IPG in some examples). In some such instances, other ECG electrodes are spaced apart from the ECG electrode associated with the stimulation support portion of stimulation element. In some examples, at least some ECG sensing electrodes also may be employed to deliver stimulation to a nerve or muscle, such as but not limited to, an upper airway patency-related nerve (e.g., hypoglossal nerve) or other nerves or muscles (e.g., phrenic nerve or diaphragm). Other examples may not include a stimulation element, such as those including a method and/or device of FIGS. 1-8C and which may not include stimulating target tissue.

In some examples, other types of sensing may be employed to obtain cardiac information (including but not limited to heart rate and/or heart rate variability), such as a cardiac sensor 2023 shown in FIG. 9C, which may comprise at least one of a ballistocardiogram sensor(s), seismocardiogram sensor(s), and/or accelerocardiogram sensor(s). In some examples, such sensing is based on and/or implemented via accelerometer-based sensing such as described above and further described below in association with accelerometer 2026.

In some examples in which the cardiac sensor 2023 comprises a ballistocardiogram sensor, the sensor 2023 may sense cardiac information caused by cardiac output, such as the forceful ejection of blood from the heart into the great arteries that occurs with each heartbeat. The sensed ballistocardiogram information may comprise HR 2025A, HRV 2025B, and/or additional cardiac morphology 2025C. In some examples such ballistocardiogram-type information may be sensed from within a blood vessel in which the sensor (e.g., accelerometer) senses the movement of the vessel wall caused by pulsations of blood moving through the vessel with each heartbeat. This phenomenon may sometimes be referred to as arterial motion.

In some examples in which the cardiac sensor 2023 comprises a seismocardiogram sensor, the sensor 2023 may provide cardiac information which is similar to that described for ballistocardiogram sensor, except for being obtained via sensing vibrations, per an accelerometer (e.g., single or multi-axis), in or along the chest wall caused by cardiac output. In particular, the seismocardiogram measures the compression waves generated by the heart (e.g., per heart wall motion and/or blood flow) during its movement and transmitted to the chest wall. Accordingly, the sensor 2023 may be placed in the chest wall.

In some such examples of sensing per sensor 2023, such methods and/or devices also may comprise sensing a respiratory rate and/or other respiration information.

In some examples the sensing portion 2000 may comprise an electroencephalography (EEG) sensor 2012 to obtain and track EEG information. In some examples, the EEG sensor 2012 may also sense and/or track central nervous system (CNS) information in addition to sensing EEG information. In some examples, the EEG sensor(s) 2012 may be implanted subdermally under the scalp or may be implanted in a head-and-neck region otherwise suitable to sense EEG information. Accordingly, the EEG sensor(s) 210 are located near the brain and may detect frequencies associated with electrical brain activity.

In some examples, a sensing element used to sense EEG information is chronically implantable, such as in a subdermal location (e.g., subcutaneous location external to the cranium skull), rather than an intracranial position (e.g., interior to the cranium skull). In some examples, the EEG sensing element is placed and/or designed to sense EEG information without stimulating a vagus nerve at least because stimulating the vagal nerve may exacerbate sleep apnea, particularly with regard to obstructive sleep apnea. Similarly, the EEG sensing element may be used in a device in which a stimulation element delivers stimulation to a hypoglossal nerve or other upper airway patency-related nerve without stimulating the vagus nerve in order to avoid exacerbating the obstructive sleep apnea.

In some examples, sensed EEG information may be used as part of (or solely in) making a sleep-wake determination, such as sleep onset, and wake onset. Among other uses, this sleep-wake information may help provide overall sleep hours, which may comprise part of therapy outcome, in some examples.

In some examples, sensed EEG information may be used to detect sleep stages during sleep. Among other uses, this sensed sleep stage may help determine an absolute amount or relative amount of deep sleep, REM sleep per night, and/or other sleep metrics. For instance, such information may be used to evaluate whether a particular therapy and/or stimulation setting corresponds to a patient's most therapeutic stimulation energy settings/parameters based on (at least or in part) the recognition more deep sleep typically corresponds to the most or more therapeutic stimulation energy settings whereas less deep sleep typically corresponds to lesser therapeutic stimulation energy settings.

In some examples, sensed EEG information may be used to detect arousals, which may comprise one aspect of determining therapy outcome. Among other uses, the detection of more arousals may provide an indication of the patient exhibiting more daytime sleepiness, which in turn may lead to adjustments to care or stimulation solution settings (e.g., values of stimulation energy parameters, and/or time or amount of mechanical maneuvering) in order to minimize arousals.

In some examples, the above-described aspects regarding the use of sensed EEG information may be combined in whole, or part, to provide an overall sleep efficiency parameter. In some such examples, the sleep efficiency parameter may be based on: 1) sleep duration; 2) sleep depth; and/or 3) events (e.g., number of arousals). In some examples, the sleep efficiency parameter may be compared to a reference sleep efficiency parameter such as (but not limited to): 1) a reference sleep duration (e.g., 8-9 hours); 2) a reference sleep depth (e.g., a minimum duration of deep sleep and REM sleep; and/or 3) few or no arousals.

In some examples the sensing portion 2000 may comprise an electromyogram (EMG) sensor 2022 to obtain and track EMG information. In some examples, the sensed EMG signals may be used to identify sleep, respiration information (e.g., respiratory phase information) and/or obstructive events. In some examples, the detected EMG information may be used to detect arousals and/or overall patient movement. These examples of determining and/or using sensed EMG information may be used as part of determining patient metrics (e.g., therapy outcome, usage, other) by which care parameters (e.g., stimulation energy parameters pr mechanical maneuvering parameters, such as timing and/or amount of mechanical maneuvering of tissue) may be determined, adjusted, etc. in order to maintain and/or improve those patient metrics according to various examples of the present disclosure.

In some examples, any one or a combination of the various sensing modalities (e.g., EEG, EMG, etc.) described in association with FIG. 9C may be implemented via a single sensing element 2014.

In some examples, the sensing portion 2000 may comprise an accelerometer 2026. In some examples, the accelerometer may comprise a single axis accelerometer while in some examples, the accelerometer may comprise a multiple axis accelerometer.

Among other types and/or ways of sensing information, the accelerometer sensor(s) 2026 may be employed to sense or obtain a ballistocardiogram, a seismocardiogram, and/or an accelerocardiogram (see cardiac sensor 2023 and related disclosure), which may be used to sense (at least) HR 2025A and/or HRV 2025B (among other information such as respiratory rate in in some instances), which may in turn may be used as part of determining respiration information, cardiac information, as described throughout the examples of the present disclosure. In some examples, this sensed information also may be used in determining sleep-wake status.

In some examples, the accelerometer 2026 may be used to sense activity, posture, and/or body position as part of determining a patient metric, the sensed activity, posture, and/or body position may sometimes be at least partially indicative of a sleep-wake status, which may be used as part of automatically initiating, pausing, and/or terminating stimulation therapy.

In some examples, the sensing portion 2000 may comprise an impedance sensor 2036, which may sense transthoracic impedance or other bioimpedance of the patient. In some examples, the impedance sensor 2036 may comprise a plurality of sensing elements (e.g., electrodes) spaced apart from each other across a portion of the patient's body. In some such examples, one of the sensing elements may be mounted on or form part of an outer surface a housing of a stimulation support portion (e.g., which may form part of 133 in FIG. 10BC) or other implantable sensing monitor, while other sensing elements may be located at a spaced distance from the stimulation support portion and/or stimulation electrode arrangement. In at least some such examples, the impedance sensing arrangement integrates all the motion/change of the body (e.g., such as respiratory effort, cardiac motion, etc.) between the sense electrodes (including the case of the IPG when present). Some examples implementations of the impedance measurement circuit include separate drive and measure electrodes to control for electrode to tissue access impedance at the driving nodes. Such impedance sensing may be used for other purposes.

In some examples, the sensing portion 2000 may comprise a pressure sensor 2037, which senses respiration information, such as but not limited to respiratory cyclical information. In some examples, the pressure sensor 2037 may be located in direct or indirect continuity with respiratory organs or airway or tissues supporting the respiratory organs or airway in order to sense respiration information.

In some examples, one sensing modality within sensing portion 2000 may be at least partially implemented via another sensing modality within sensing portion 2000.

In some examples, sensing portion 2000 may comprise an acoustic sensor 2039 to sense acoustic information, such as but not limited to cardiac information (including heart sounds), respiration information, snoring, etc.

In some examples, sensing portion 2000 may comprise body motion parameter 2035 by which patient body motion may be detected, tracked, etc. The body motion may be detected, tracked, etc. via a single type of sensor or via multiple types of sensing. For instance, in some examples, body motion may be sensed via accelerometer 2026 and in some examples, body motion may be sensed via EMG 2022 and/or other sensing modalities.

In some examples, the sensing portion 2000 in FIG. 9C may comprise a body position/posture parameter 2042 and/or body motion parameter 2035 to sense and/or track sensed information regarding posture, which also may comprise sensing of body position, activity, etc. of the patient. This sensed information may be indicative of an awake or sleep state of the patient in some examples. In some such examples, such information may be sensed via accelerometer 2026 as mentioned above, and/or other sensing modalities. In some examples, such posture information (and/or body position, activity) may be used sometimes alone and/or in combination with other sensing information to determine a patient metric. As described elsewhere herein, in some examples posture may be considered as one of several parameters when determining a probability of sleep (or awake). In some such examples, the sleep-wake status may be used to initiate, pause, and/or terminate therapy (e.g., mechanical maneuvering and/or stimulation) within a nightly treatment period.

In addition or alternatively, sensing activity, motion, and/or body position (e.g., posture) may be used to track a relative degree to which a patient is more active or less active during daytime hours, which may comprise one objective measure of therapy outcome because if the patient is sleeping better at night due to a desirable care settings (e.g., mechanical maneuvering settings and/or stimulation solution settings, such as values of stimulation energy parameters) which better control SDB, the patient may be much more active during daytime (non-sleep) hours as compared to a baseline in which their sleep disordered breathing was poorly controlled (corresponding to mechanical maneuvering settings and/or inferior stimulation energy settings) or not controlled at all. Similarly, sensing activity and/or motion as described herein also may be used to detect if the patient tends to falls asleep during daytime (e.g., non-sleep) hours, which may be an objective therapy outcome parameter by which stimulation energy parameters (and associated usage, and other therapy outcome parameters) and/or mechanical maneuvering parameters may be evaluated and potentially adjusted. This objective therapy outcome information also may be used in conjunction with subjective therapy outcome information such as, but not limited to, the Epworth Sleepiness Scale (ESS) and/or other forms of patient input regarding the patient's perceived daytime sleepiness, daytime functional ability, perceived sleep quality, etc.

In some examples, the sensing portion 2000 may comprise an other parameter 2041 to direct sensing of, and/or receive, track, evaluate, etc. sensed information other than the previously described information sensed via the sensing portion 2000.

As further shown in FIG. 9C, in some examples the sensing portion 2000 may comprise a temperature sensor 2038. In some example methods, sensing a change in temperature (such as via sensor 2038) during a treatment period may be used to identify SDB behavior. In some such examples, additional sensed information (as described in examples of the present disclosure) may be used in addition to sensed temperature to identify sleep SDB behavior. In some examples, smaller yet detectable temperature changes within a treatment period may be used to at least partially determine a patient metric. For instance, a detectable temperature change may be sensed as a result of patient exertion to breathe in response to an apnea event, given the greater muscular effort in attempting to breathe.

In some examples, at least some of the sensors and/or sensor modalities described in association with FIG. 9C may be incorporated within or on a stimulation element (e.g., 201 in FIG. 10BB) which comprise at least some implantable components, in some examples.

FIGS. 10A-10E illustrate example IMDs which may be used in addition to the implantable traction apparatus. Example IMDs may be used to stimulate nerves and/or muscles. In some examples, the IMDs illustrated by FIGS. 10A-10E may be used in addition to any of the implantable traction apparatuses illustrated and described in connection with FIGS. 3A-6C and/or FIGS. 8A-8C, such as illustrated by the implantable traction apparatus (ITA) 2027 in FIG. 10A. In some examples, the ITA 2027 and/or other IMDs may be used in addition to an external breathing therapy device (EBTD), such as the EBTD 2029 in FIG. 10A. In addition, in some examples, the above-noted implantable medical devices (e.g., sensing, therapeutic, other) which may act in complementary relation to the ITA 2027 and/or EBTD 2029 may be implemented with at least some external components, and in some variations, may be implemented entirely via external components as more fully described later in association with the example arrangement 3100 of FIG. 15.

More specifically, FIG. 10A is diagram including a front view schematically representing deployment of an example IMD 2022, which includes at least one sensor 2025. As shown in FIG. 10A, in some examples the IMD 2022 (and therefore the at least one implantable sensor 2025) may be chronically implanted in a pectoral region 2031 of a patient 2035. The at least one implantable sensor 2025 may sense data indicative of various physiologic phenomenon sensed from this implanted position (e.g., body motion, posture, vibrations, such as anatomy vibrations and device vibrations). In some examples, the IMD 2022 may comprise an implantable pulse generator (IPG), such as for managing sensing and/or stimulation therapy.

FIG. 10B is a block diagram schematically representing one example of an IMD 2051. The IMD 2051 may include a stimulation element. The stimulation element may include an IPG assembly 2063 and at least one stimulation lead 2055. The IPG assembly 2063 may include a housing 2060 containing circuitry 2062 and a power source 2064 (e.g., battery), and an interface block or header-connector 2066 carried or formed by the housing 2060. The housing 2060 is configured to render the IPG assembly 2063 appropriate for implantation into a human body, and may incorporate biocompatible materials and hermetic seal(s). The circuitry 2062 may be implemented, at least in part, via a control portion (and related functions, portions, elements, engines, parameters, etc.) such as described later in connection with at least FIGS. 11A-15.

In some examples, the stimulation lead 2055 includes a lead body 2080 with a distally located stimulation electrode arrangement 2082. At an opposite end of the lead body 2080, the stimulation lead 2055 includes a proximally located plug-in connector 2084 which is configured to be removably connectable to the interface block 2066. For example, the interface block 2066 may include or provide a stimulation port sized and shaped to receive the plug-in connector 2084.

In general terms, the stimulation electrode arrangement 2082 may optionally be a cuff electrode, and may include some non-conductive structures biased to (or otherwise configurable to) releasable secure the stimulation electrode 2082 about a target nerve. Other formats are also acceptable. Moreover, the stimulation electrode arrangement 2082 may include an array of contact electrodes to deliver a stimulation signal to a target nerve. Examples are not limited to cuffs and may include stimulation elements having a stimulation electrode arrangement 2082 in different types of arrangements and/or for different targets, such as an alternating current (AC) target, a paddle, and an axial arrangement, among others.

In some examples, the lead body 2080 is a generally flexible elongate member having sufficient resilience to enable advancing and maneuvering the lead body 2080 subcutaneously to place the stimulation electrode arrangement 2082 at a desired location adjacent a nerve, such as an upper airway patency-related nerve (e.g., hypoglossal nerve, nerves innervating various infrahyoid strap muscles). In some examples, such as in the case of OSA, the nerves may include (but are not limited to) the nerve and associated muscles responsible for causing movement of the tongue and related musculature to restore airway patency. In some examples, the nerves may include (but are not limited to) the hypoglossal nerve and the muscles may include (but are not limited to) the genioglossus muscle. In some examples, lead body 2080 may have a length sufficient to extend from the IPG assembly 2063 implanted in one body location (e.g., pectoral) and to the target stimulation location (e.g., head, neck). Upon generation via the circuitry 2062, a stimulation signal is selectively transmitted to the interface block 2066 for delivery via the stimulation lead 2055 to the nerves.

It will be understood that the interface block 2066 is representative of many different kinds and styles of electrical (and mechanical) connection between the housing of the IPG assembly 2063 and the lead 2055 with such connections having a size, shape, location, etc. which may differ from the interface block 2066 shown in FIG. 10B.

In some examples, the IMD 2051 includes at least one implantable sensor 2025 may be connected to the IMD 2051 in various fashions, such as being connected to the interface block 2066, being carried by (or within) the IPG assembly 2063, and/or wirelessly communicating with the IPG assembly 2063. More specifically, the at least one implantable sensor 2025 may be connected in various orientations as described within U.S. Patent Publication No. 2021/0268279, published on Sep. 2, 2021, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety. Although the above examples describe an IMD 2051 having a stimulation lead 2055, examples are not so limited and example IMDs may additionally or alternatively include a lead used for sensing.

It will be understood that the example IMDs in FIGS. 10A-10E are not limited to the example sensors described in association with FIGS. 10A-10E but may comprise at least one of the different sensor modalities, placements, etc., described in association with at least FIGS. 9A-9B, FIG. 15, and/or generally throughout the present disclosure.

In some examples, the at least one implantable sensor 2025 may be wirelessly connected to the IMD 2051. In such examples, the interface block 2066 need not provide a sense port for the at least one implantable sensor 2025 or the sense port may be used for a second sensor. In some examples, the circuitry 2062 of the IPG assembly 2063 and circuitry of the at least one implantable sensor 2025 communicate via a wireless communication pathway according to known wireless protocols, such as Bluetooth, near-field communication (NFC), Medical Implant Communication Service (MICS), 802.11, etc. with each of the circuitry 2062 and the at least one implantable sensor 2025 including corresponding components for implementing the wireless communication pathway. In some examples, a similar wireless pathway is implemented to communicate with devices external to the patient's body for at least partially controlling the at least one implantable sensor 2025 and/or the IPG assembly 2063, to communicate with other devices (e.g., other sensors) internally within the patient's body, or to communicate with other sensors external to the patient's body.

FIG. 10BB is a block diagram schematically representing an example device which may be used to implement the method 10 of FIG. 1, mechanically maneuver the thyroid cartilage and/or the hyoid bone, and/or to electrically stimulate target tissue. In some examples, the device 215 may form part of any of the devices illustrated by FIGS. 3A, 4A-4C, and 5A-8C and/or may be in communication with any of the devices of FIGS. 3A, 4A-4C, and 5A-8C (e.g., control portion of device 215 may communicate therewith or form part of the devices). In some examples, the device 215 may form part of any of the devices illustrated by FIGS. 10A and 10C-10E. For example, the activation element 201 may form part of devices and/or be used to stimulate target tissue 213 in addition to mechanically maneuvering the thyroid cartilage and/or the hyoid bone in accordance with the methods, devices, and/or techniques as described in connection with any of FIGS. 1-9C. In some examples, the activation element 201 may include or be in communication with control portion 1916 of FIG. 9B, sensing portion 2000 of FIG. 9C, control portion 2100 of FIG. 11A, care portion 2500 of FIG. 14, or external element 3150 of FIG. 15.

As shown in FIG. 10BB, in some examples, a device 215 may comprise an activation element 201. In some examples, the activation element 201 may be a stimulation element that includes a stimulation electrode arrangement, such as at least one stimulation electrode. In some examples, the activation element 201 may further include a lead that supports the at least one stimulation electrode (e.g., of a stimulation electrode arrangement) of the activation element 201 and includes a stimulation support portion (e.g., 133 in FIG. 10BC). As described in association with at least FIG. 10BC, among other example implementations, one example implementation of a stimulation support portion 133 may comprise stimulation (or control) circuitry, which may be embodied as a PG. Further example implementations of a stimulation support portion may comprise a sensing element to perform sensing and/or to receive sensed data from sensors external to the stimulation element (e.g., including being external to the stimulation support portion), with such sensors being implantable and/or external to the body. In some examples, the sensor(s) may comprise at least some of substantially the same features as described throughout FIGS. 1-10B and FIGS. 10C-19, with particular reference to sensor portion 2000 of FIG. 9C and/or external element 3150 in FIG. 15.

With further reference to FIG. 10BB, in some examples, the activation element 201 (which is or includes a stimulation element) may form part of a catheter or lead which is placed within the body. Various types of leads may be used, including but not limited to, a spiral-type lead, a basket or lasso type lead, and a lead with tined tips, among others. In general terms, in some examples, the activation element 201 (or at least a portion thereof) is located at a position adjacent to target 213 such as (but not limited to) a hypoglossal nerve (or other upper airway patency-related tissue) such that stimulation applied via the activation element 201 is delivered to the hypoglossal nerve. Via this example arrangement, the stimulation element 201 becomes positioned into stimulating relation to the target tissue 213. In some examples, “stimulating relation” may include and/or refer to an activation element 201 which is a stimulation element (e.g., includes at least one electrode) being in a position, orientation, and/or distance such that the applied stimulation signal provides at least some capture of a nerve (e.g., at least tone response of muscle, and, in some instances, supra-threshold or full muscle contraction) and/or of a muscle. In some instances, the stimulation may be tonic stimulation.

In some examples, activation element 201 may comprise at least one stimulation electrode(s) which may take a wide variety of forms, and may be incorporated within a wide variety of different types of stimulation electrode arrangements, at least some of which are described in association with at least FIGS. 10A-10E. In some examples, the activation element 201 includes a pair of electrodes or a plurality of pairs of electrodes. In some examples, the activation element 201 includes a plurality of ring electrodes. In other examples, the activation element 201 includes a planar electrode or a plurality of planar electrodes. In some examples, the stimulation applied may be bipolar or monopolar.

In some examples, the electrode(s) of the activation element 201 used for applying stimulation also may be used for sensing, but not necessarily for simultaneous stimulation and sensing. However, in some examples, the electrode(s) of the activation element 201 are used solely for applying stimulation while some electrode(s) may be used solely for sensing.

In some examples, the device 215 may be implanted within the patient's body. For example, the activation element 201, or at least a portion thereof, may be inserted within the patient's body and maneuvered to the target location for applying stimulation to the target tissue 213. In some examples and as noted above, the activation element 201 of the device 215 may further include a lead that supports the at least one stimulation electrode of the activation element 201.

In some examples, as noted above, the activation element 201 may further include a stimulation support portion (e.g., at least 133 in FIG. 10BC) which may be embodied as a pulse generator (PG), such as illustrated in connection with at least FIGS. 10A-10B. In some such examples, the entire PG (and/or other power, control, and/or communication elements) may be implantable while in some examples, some portions of the PG (and/or other power, control, and/or communication elements) may be external to the patient as further described in association with at least FIG. 15. In some examples, the IPG or a non-implanted PG may be separate from the stimulation electrode arrangement. In some examples, the PG may be located within the head-and-neck region or the pectoral region of the patient. In some examples, the IPG may be chronically implanted in at least one of the torso region, the neck region, or the cranial region. The torso region may include the sternum, pectoral region, or other areas. In some examples, the neck region may include the neck and other areas, such as a transitional area of the neck (e.g., between the neck and torso and/or between neck and cranial region) including the clavicle, manubrium (e.g., at the top of sternum), and mandible. In some examples, the cranial region may include the skull, such as behind the ear of the patient, among other locations. In some examples, components may be implanted in the cranial region or in the head region, which may be referred to as a “head-and-neck region” for ease of reference.

In some examples, the activation element 201 may include a stimulation support portion, such as further described herein in connection with at least FIG. 10BC. As further described herein, in some examples the stimulation support portion may be implemented as a PG such as (but not limited to) an IPG.

As shown in FIG. 10BC, in some examples, the stimulation support portion 133 may comprise stimulation function circuitry 134A (e.g., for controlling stimulation, mechanical maneuvering, or other therapy), a power element 134B, a sensing element 134C, a control element 134D, a communication element 134E (e.g., at least a receiver), and/or other element 134F.

In some examples, the stimulation function circuitry 134A may comprise passive stimulation circuitry, e.g., circuitry which does not generate a stimulation signal but which may receive a stimulation signal generated elsewhere (e.g., external of the patient or from an implanted device) and which is then communicated (e.g., via lead) to the electrodes of the stimulation electrode arrangement for stimulating the target tissue 213 (e.g., upper airway patency-related tissue or other tissue).

With further reference to the particular example illustrated in FIG. 10BC, in some examples, the stimulation function circuitry 134A comprises active stimulation circuitry, e.g., components sufficient to generate a stimulation signal within the stimulation support portion 133 for transmission (e.g., via a lead or other means) to the electrodes of the stimulation electrode arrangement of the stimulation element (e.g., activation element 201 of FIG. 10BB). In some such examples, the stimulation support portion 133 may sometimes comprise and/or be referred to as a PG. Moreover, in some such examples, given the stimulation support portion 133 being sized and shaped for implantation in the head-and-neck region, the stimulation support portion 133 may sometimes be referred to as a microstimulator.

Whether referred to as a microstimulator or not, in these examples the housing 135 of the stimulation support portion 133 may sealingly contain (e.g., encapsulate) the stimulation function circuitry 134A, along with other elements such a power element 134B, communication element 134E, and/or control element 134D, among other potential components (e.g., sensing 134C, etc.).

In some examples, the stimulation support portion 133 of the activation element 201 may comprise a power element 134B. The power element 134B may be non-rechargeable, in some examples. However, the power element 134B may be re-chargeable in some examples such that the power element 134B receives power from a power source external of the stimulation support portion 133, with the power source being implantable in some examples or being external of the patient in some examples. For instance, the power element 134B may receive power via a wired connection (e.g., in some examples in which the power source is implantable) or via wireless communication, in which the power source may be implantable or external to the patient. In some examples in which the power source may be external to the patient, the power source may comprise at least some of substantially the same features and attributes as external power portion 3174 in FIG. 15, as further described below.

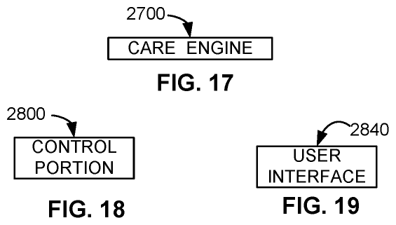

In some examples, the stimulation support portion 133 comprises a control element 134D which provides on-board control of at least some of the functions of the activation element 201 (including stimulation electrode arrangement, stimulation support portion 133 and/or other components of the activation element 201). In some examples, the control element 134D may comprise the entire control portion for the activation element 201. In some examples, the control element 134D may form part of a larger control portion in which the control element 134D may receive at least some control signals from components of the control portion external to the stimulation support portion 133. In some such examples, these components of the control portion which are external to the stimulation support portion 133 also may be external to the patient. For example, the control element 134D of stimulation support portion 133 may comprise at least a partial implementation of, and/or communicate with, a control portion 1916 of FIG. 9B, control portion 2100 or 2120 of FIGS. 11A-11B, care portion 2500 of FIG. 14, care engine 2700 of FIG. 17, and/or control portion 2800 of FIG. 18. As such, consistent with the later described control portion 2100 of FIG. 11A, the control element 134D in FIG. 10BC also may comprise a memory to store stimulation therapy information (e.g., therapy settings, usage, outcomes, etc.), control information, sensed information (per sensing element 1034C), etc.

In some examples, the sensing element 134C of stimulation support portion 133 may store data sensed by an on-board sensor of the activation element 201 and/or sensed via sensor external to the stimulation element 201 (e.g., external to stimulation support portion 133, stimulation electrode arrangement) with such sensor (external to the activation element 201) being implantable or external to patient. In some examples, an on-board sensor may comprise an accelerometer (e.g., tri-axis), gyroscope, etc. In some examples, such on-board sensor may comprise an electrode exposed on surface of housing, which in combination with other electrodes may be used to sense impedance and/or other biosignals. With these brief examples in mind, it will be understood that in some examples the sensing element 134C may comprise, and/or receive sensed information from, at least some of substantially the same sensing elements, functions, etc. as described in association with at least FIG. 9B (e.g., control portion 916), FIG. 9C (e.g., sensing portion 2000), FIG. 11A (e.g., control portion 2100, care engine 2109), FIG. 14 (e.g., care portion 2500), and/or FIG. 15 (e.g., external element 3150).

In some examples, the stimulation support portion 133 of the activation element 201 may comprise a communication element (e.g., coil, antenna, and any related circuitry) to transmit and/or receive the control information, therapy data, sensed data, and the like. In addition to, or instead of these examples, the communication element may be configured to facilitate receive power from a power source(s) external to the stimulation support portion 133, whether via wired connection or wirelessly. In some examples, the communication element 134E may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), ultrasound, and/or other short range or long range wireless communication protocols suitable for use in communicating between implanted components within the body and/or communicating between implanted components and external components in a medical device environment.

It will be understood that in some examples of the present disclosure, a lead may be omitted and at least some of the operative components of the stimulation support portion 133 may be incorporated into and/or with the stimulation electrode arrangement, such as illustrated by (but not limited to) the example stimulation electrode arrangement 2412 of FIG. 10D. In some such examples, the stimulation electrode arrangement may sometimes comprise, or be referred to as, a leadless stimulation electrode arrangement or a leadless activation element 201. In some of these examples, the functions and/or components of the stimulation support portion 133 which are incorporated into the stimulation electrode arrangement may comprise passive stimulation circuitry (which may be embodied as a part of the communication element 134E) to receive a stimulation signal generated elsewhere and conduct this stimulation signal to the electrodes of the stimulation electrode arrangement.

Referring back to FIG. 10BB, in some examples, portions of the activation element 201 may comprise anchor elements or other structures which act to maintain at least the stimulation electrode arrangement in a selected location to maintain at least some electrodes of the activation element 201 in stimulating relation to target tissue. Accordingly, in some such examples, the housing of stimulation support portion (133 of FIG. 10BC) may comprise an array of anchor elements, which may comprise tines, barbs, and/or other tissue-engaging structures to hinder or prevent movement of the housing relative to target tissue in which the stimulation support portion is present. In some examples, with or without such tines, barbs, etc., a shape of the housing of the stimulation support portion may act as, and/or form part of, an anchor arrangement. Similarly, in some examples, portions of the activation element 201 may comprise anchor elements or other structures which act to maintain at least contact between the implantable traction apparatus and the tissue.

FIG. 10C is a diagram including a front view of an example IMD 2411 (and/or example method) implanted within a patient's body 2410. In some examples, the IMD 2411 may comprise (but is not limited to) at least one stimulation element. The stimulation element may comprise an IPG 2433 implanted in a pectoral region 2401. In some examples, the IMD 2411 further includes a sensor 2435. In some examples, IMD 2411 includes an implementation of, and/or at least some of substantially the same features and attributes as, the IMD 2022 as previously described in connection with at least FIG. 10B). Accordingly, in some examples, sensor 2435 may comprise at least an acceleration sensor having at least some of substantially the same features and attributes as previously described in connection with at least FIGS. 9A-9C. Via such example sensing arrangements, a control portion (which may comprise part of, and/or be in communication with the actuator and/or IMD 2411) may identify the body posture, SDB obstruction or event, respiration information, cardiac information, sleep state and/or other parameters. For example, FIG. 10C illustrates an example of at least one sensor by which FIGS. 9A and/or 9A may be implemented. Among other features, it will be understood that in some examples a body of a lead 2417 supports the stimulation electrode arrangement 2412, while extending between (and connecting) the IPG 2433 and the stimulation electrode arrangement 2412. Moreover, in some examples, the IPG 2433 may be formed on a smaller scale (e.g., microstimulator) and/or different shape to be amenable for implantation in a head and/or neck region 2403 instead of pectoral region 2401.

As further shown in FIG. 10C, IMD 2411 comprises a lead 2417 including a lead body 2418 for chronic implantation (e.g., subcutaneously via tunneling or other techniques) and to extend to a position adjacent a nerve (e.g., hypoglossal nerve 2405, and/or other infrahyoid muscle-innervating nerve 2406). The lead 2417 may support the stimulation electrode arrangement 2412 to engage the nerve (e.g., 2405, 2406) in a head-and-neck region 2403 for stimulating the nerve to treat a physiologic condition, such as SDB. The IMD 2411 may comprise circuitry, power element, etc., to support control and operation of the sensor 2435 and the stimulation electrode arrangement 2412 (via lead 2417). In some examples, the control, operation, etc., may be implemented, at least in part, via a control portion (and related functions, portions, elements, engines, parameters, etc.) as described later in connection with at least FIGS. 11A-19.

With regard to the various examples, delivering stimulation to an upper airway patency-related nerve 2405 (e.g., a hypoglossal nerve, infrahyoid muscle-innervated nerve) via the stimulation electrode arrangement 2412 is to cause contraction of upper airway patency-related muscles, which may cause or maintain opening of the upper airway (2408) to prevent and/or treat OSA.

As further shown in the diagram of FIG. 10C, in some examples, IMD 2411 may be implemented with additional sensors 2420, 2430 to sense bio-impedance, ECG, and/or other sensing modalities, from which additional physiologic data, such as, but not limited to, further respiration information, cardiac information, etc. In some examples, one or both of the sensors 2420, 2430 may comprise sensor electrodes. In some examples, stimulation electrode arrangement 2412 also may act, in some examples, as a sensing electrode. In some examples, at least a portion of housing of the IPG 2433 also may comprise a sensor or at least an electrically conductive portion (e.g., electrode) to work in cooperation with at least some of the above-mentioned sensing electrodes to implement at least some sensing arrangements to sense bioimpedance, ECG, etc., as described above.

FIG. 10D is a diagram schematically representing an example IMD 2419A comprising at least some of substantially the same features and attributes as the IMD 2411 in FIG. 10C, except with the IPG 2433 implemented as a microstimulator 2419B. In some examples, the microstimulator 2419B may be chronically implanted (e.g., percutaneously, subcutaneously, transvenously, etc.) in a head-and-neck region 2403 as shown in FIG. 10D, or in a pectoral region 2401. In some examples, as part of the IMD 2419A, the microstimulator 2419B may be in wired or wireless communication with stimulation electrode arrangement 2412. In some examples, as part of the IMD 2419A, the microstimulator 2419B also may incorporate sensor 2435 or be in wireless or wired communication with a sensor 2435 located separately from a body of the microstimulator 2419B. When wireless communication is employed for sensing and/or stimulation, the microstimulator 2419B may be referred to as leadless implantable medical device for purposes of sensing and/or stimulation. In some examples, the microstimulator 2419B may be in close proximity to a target nerve 2405.

In some examples, the microstimulator 2419B (and associated elements) and/or IMD 2419A may comprise at least some of substantially the same features and attributes as described in U.S. Patent Publication No. 2020/0254249, published on Aug. 13, 2020, and entitled “MICROSTIMULATION SLEEP DISORDERED BREATHING (SDB) THERAPY DEVICE”, the entire teachings of which is incorporated herein by reference in its entirety.

FIG. 10E is a diagram schematically representing an example IMD 2413 comprising at least some of substantially the same features and attributes as the IMD 2411 in FIG. 10C and the IMD 2419A in FIG. 10C, such that the IMD 2413 includes both an IPG 2433 implanted in a pectoral region 2401 and a microstimulator 2419B implanted in the head-and-neck region 2403. In some examples, the IMD 2413 of FIG. 10E includes an implementation of, and/or at least some of substantially the same features and attributes, as the IMDs of any of FIGS. 10A-10D. The common features and attributes are not repeated. In some examples, the IPG 2433 may be used to stimulate one type of tissue (e.g., upper airway patency-related tissue) and the microstimulator 2419B may be used to stimulate another type of tissue (e.g., phrenic nerve, diaphragm, other), or vice versa. In some examples, the IPG 2433 may to stimulate one type of upper airway patency-related tissue (e.g., hypoglossal nerve, genioglossus muscle) and the microstimulator 2419B may be used to stimulate another type of upper airway patency-related tissue (e.g., infrahyoid muscle-innervating nerve, infrahyoid muscles), or vice versa.

As implicated by the above description, one or both of the actuator of the implantable traction apparatus, any other medical devices (MD), whether including implantable or external components) include a controller, control unit, or control portion that prompts, controls, tracks, evaluates, etc., performance of designated actions.

FIG. 11A is a block diagram schematically representing an example control portion. In some examples, the control portion 2100 includes a controller (e.g., processor) 2102 and a memory 2104. In some examples, the control portion 2100 provides one example implementation of a control portion forming a part of, implementing, and/or managing any one of devices, actuators, electrodes (e.g., coupled to gel or piezoelectric material), assemblies, circuitry, managers, engines, functions, parameters, respiration determination elements, stimulation elements, PGs, sensors, electrodes, modules, and/or methods, as represented throughout the present disclosure in association with FIGS. 1-10E and FIGS. 11B-15.

The control portion 2100 may include circuitry components and wiring appropriate for generating desired stimulation signals (e.g., converting energy provided by the power source into a desired stimulation signal), for example in the form of the care engine 2109. In some examples, the control portion 2100 may include telemetry components for communication with external devices. For example, the control portion 2100 may include a transmitter that transforms electrical power into a signal associated with transmitted data packets, a receiver that transforms a signal into electrical power, a combination transmitter/receiver (or transceiver), an antenna (e.g., an inductive telemetry antenna), etc.

In general terms, the controller 2102 of the control portion 2100 comprises an electronics assembly 2106 (e.g., at least one processor, microprocessor, integrated circuits and logic, etc.) and associated memories or storage devices. The controller 2102 is electrically couplable to, and in communication with, the memory 2104 to generate control signals to direct operation of at least some the devices, actuators, electrodes, assemblies, circuitry, managers, modules, engines, functions, parameters, respiration determination elements, stimulation elements, PGs, sensors, electrodes, and/or methods, as represented throughout the present disclosure. In some examples, these generated control signals include, but are not limited to, employing the mechanically maneuvering by the actuator of the implantable winch device, implementing therapy via implantable and/or external therapy devices, related sensing, or combinations thereof. The control signals may be a software program stored on the memory 2104 (which may be stored on another storage device and loaded onto the memory 2104), and executed by the electronics assembly 2106. In some examples, the control signals also may at least identify information regarding respiration, upper airway obstruction (which may come from respiration information in some examples), cardiac body position, sleep state, and/or other physiologic (or other) phenomenon. In addition, and in some examples, these generated control signals include, but are not limited to, employing the care engine 2109 stored in the memory 2104 to at least manage care provided to the patient, for example therapy for SDB (and/or other therapies, such as cardiac), with such care in at least some examples including providing mechanical maneuvering of the thyroid cartilage and/or hyoid bone.

In response to or based upon commands received via a user interface (e.g., user interface 2240 in FIG. 12), sensor signals, and/or via machine readable instructions, controller 2102 generates control signals as described above in accordance with examples of the present disclosure. In some examples, controller 2102 is embodied in a general purpose computing device while in some examples, controller 2102 is incorporated into or associated with at least some of the sensors, sensing element, respiration determination elements, actuator elements, electrodes, stimulation elements, PGs, actuators, devices, user interfaces, instructions, information, engines, functions, actions, and/or method, etc. as described throughout examples of the present disclosure.

For purposes of this application, in reference to the controller 2102, the term “processor” shall mean a presently developed or future developed processor (or processing resources) that executes machine readable instructions contained in a memory. In some examples, execution of the machine readable instructions, such as those provided via memory 2104 of control portion 2100 cause the processor to perform the above-identified actions, such as operating controller 2102 to implement the sensing, monitoring, identifying the upper airway obstruction, mechanical maneuvering, and/or treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 2104. In some examples, the machine readable instructions may comprise a sequence of instructions, or the like. In some examples, memory 2104 comprises a computer readable tangible medium providing non-volatile storage of the machine readable instructions executable by a processor of controller 2102. In some examples, the computer readable tangible medium may sometimes be referred to as, and/or comprise at least a portion of, a computer program product. In some examples, hard wired circuitry may be used in place of or in combination with machine readable instructions to implement the functions described. For example, controller 2102 may be embodied as part of at least one application-specific integrated circuit (ASIC), at least one field-programmable gate array (FPGA), and/or the like. In some examples, the controller 2102 is not limited to any specific combination of hardware circuitry and machine readable instructions, nor limited to any particular source for the machine readable instructions executed by the controller 2102.

In some examples, control portion 2100 may be entirely implemented within or by a stand-alone device.

In some examples, the control portion 2100 may be partially implemented in one of the sensors, sensing element, actuator elements, respiration determination elements, monitoring devices, stimulation devices, etc. and partially implemented in a computing resource (e.g., at least one external resource) separate from, and independent of, the implantable traction apparatus (or portions thereof, and/or other medical devices) but in communication with the implantable traction apparatus (or portions thereof, and/or other medical devices). For instance, in some examples, control portion 2100 may be implemented via a server accessible via the cloud and/or other network pathways. In some examples, the control portion 2100 may be distributed or apportioned among multiple devices or resources such as among a server, an apnea treatment device (or portion thereof), and/or a user interface.

In some examples, control portion 2100 includes, and/or is in communication with, a user interface 2240 as shown in FIG. 12.

FIG. 11B is a diagram schematically illustrating at least some example arrangements of a control portion by which the control portion 2100 (FIG. 11A) may be implemented. In some examples, control portion 2120 is entirely implemented within or by an actuator 2125, which has at least some of substantially the same features and attributes as an actuator, as previously described throughout the present disclosure. In some examples, control portion 2120 is entirely implemented within or by a remote control 2130 (e.g., a programmer) external to the patient's body, such as a patient control 2132 and/or a physician control 2134. In some examples, the control portion 2120 is partially implemented in the actuator 2125 and partially implemented in the remote control 2130 (at least one of patient control 2132 and physician control 2134).

FIG. 12 is a block diagram schematically representing a user interface. In some examples, user interface 2240 forms part of and/or is accessible via a device external to the patient and by which the implantable traction apparatus and/or other medical device may be at least partially controlled and/or monitored. The external device which hosts user interface 2240 may be a patient remote (e.g., 2132 in FIG. 11B), a physician remote (e.g., 2134 in FIG. 11B) and/or a clinician portal. In some examples, user interface 2240 comprises a user interface or other display that provides for the simultaneous display, activation, and/or operation of at least some of the sensors, sensing element, respiration determination elements, stimulation elements, actuators, PGs, devices, user interfaces, instructions, information, modules, engines, functions, actions, and/or method, etc., as described in connection with FIGS. 1-11B. In some examples, at least some portions or aspects of the user interface 2240 are provided via a graphical user interface (GUI), and may comprise a display 2244 and input 2242.

FIG. 13 is a block diagram 2350 which schematically represents some example implementations by which a device may communicate wirelessly with external circuitry outside the patient. As described above, the controller and/or control portion of at least one device 2360 (e.g., implantable in some examples) illustrated in FIG. 13 may be implemented by components of the device 2360, components of external devices (e.g., mobile device 2370, patient remote control 2374, a clinician programmer 2376, and a patient management tool 2380), and various combinations thereof. The device 2360 may include an implantable traction apparatus, implantable winch MD, as described above, and/or the device 2360 may comprise at least some aspects of a medical device implemented as further described later in association with at least FIGS. 14-15. As shown in FIG. 13, in some examples, the device 2360 may communicate with at least one of patient application 2372 on a mobile device 2370, a patient remote control 2374, a clinician programmer 2376, and a patient management tool 2380. The patient management tool 2380 may be implemented via a cloud-based portal 2383, the patient application 2372, and/or the patient remote control 2374. Among other types of data, these communication arrangements enable the implantable device 2360 to communicate, display, manage, etc., the therapy provided, as well as to allow for adjustment to the various elements, portions, etc. of the example devices and methods if and where desired. In some examples, the various forms of therapy provided may be displayed to a patient and/or clinician via one of the above-described external devices.

FIG. 14 is a block diagram schematically representing a care engine 2500 of a control portion. In some examples, the care engine 2500 may comprise an example implementation of, and/or at least some of substantially the same features and attributes as, any of the actuators, PGs, IPGs, care engines and/or the control portions (e.g., FIGS. 1-13) of the present disclosure. Accordingly, the various functions and parameters of the care engine 2500 may be implemented in a manner supportive of, and/or complementary with, the various functions, parameters, portions, etc., of any of the devices and control portions and/or various functions, parameters, portions, etc., relating to stimulation throughout examples of the present disclosure. In some examples, the care engine 2500 may include an implementation of the care engine 2109 of FIG. 11A and/or care engine/control portion 1916 in FIG. 9B. In some examples, the care engine 2500 may be implemented by and/or include a portion of the stimulation portion 3174 in FIG. 15.

In some examples, different target tissue may be stimulated using at least one stimulation element and/or mechanically maneuvered (e.g., pulling the thyroid cartilage and/or hyoid bone). The target tissue may be stimulated and/or mechanically maneuvered at the same time (e.g., simultaneously or overlapping times) or at different times and/or in response to different sensed parameters, such as those described and illustrated in connection with at least FIGS. 9A-C.

In some examples, any of the methods, apparatuses, and/or devices may be used to provide SDB care to different target tissue, including those described in connection with at least FIGS. 1-10E. For example, the method 10 of FIG. 1 may further comprise performing at least one of: (i) stimulating at least one of upper airway patency-related tissue, (ii) stimulating at least one diaphragm-related tissue, and (iii) activating an external breathing therapy device. Example upper airway patency-related tissue includes the hypoglossal nerve and/or muscles innervated by the hypoglossal nerve, infrahyoid muscle-related nerve(s), and infrahyoid strap muscles, among other tissue. The combination of stimulating upper airway patency-related tissue, mechanically maneuvering the thyroid cartilage and/or hyoid bone, and/or activating the external breathing therapy device may be used to treat OSA as well as multiple types of SDB when such upper airway patency-related tissue therapies are combined with the stimulation of diaphragm-related tissue (e.g., phrenic nerve and/or diaphragm), which is used to treat central sleep apnea (CSA). In some examples, other nerves and/or tissue may be stimulated or treated.

As shown by FIG. 14, in some examples, via target tissue parameter 2510, stimulation may be delivered to and/or mechanically maneuvering may be applied to select target tissue such as, but not limited to, thyroid cartilage, hyoid bone, upper airway patency-related tissue, and/or diaphragm-related tissue.

In some examples, the upper airway patency-related tissue may comprise a hypoglossal nerve and/or muscle (e.g., genioglossus muscle) innervated by the hypoglossal nerve to cause contraction of at least the protrusion muscles and to thereby cause protrusion of the tongue to increase and/or maintain upper airway patency. In some examples, the upper airway patency-related tissue may comprise infrahyoid strap muscles and/or infrahyoid muscle-related nerves which includes a nerve(s) innervating at least one infrahyoid strap muscle (e.g., thyrohyoid, omohyoid, sternohyoid, and/or sternothyroid), and may include an ansa cervicalis-related nerve. The infrahyoid muscle-related nerves may sometimes be referred to as infrahyoid muscle-innervating nerves. In some examples, target tissues may include any other muscles which affect and/or promote upper airway patency, and/or nerves which innervate such muscles. In some examples, target tissue includes a combination of nerves and/or muscles such as, but not limited to, terminal fiber ends of nerves where a nerve ending terminates into (or at) the muscle being innervated.

In some examples, in addition to or instead of selecting different tissue for stimulation and/or for mechanical maneuvering, the target tissue parameter 2510 may comprise adjusting care parameters (e.g., stimulation parameters) via selecting between (or using a combination of) various locations along a nerve such as stimulating multiple different sites along a particular nerve.

In some examples, in addition to or instead of selecting different nerves for stimulation and/or for mechanical maneuvering, the target tissue parameter 2510 may comprise adjusting care parameters via selecting between (or using a combination of) different fascicles within a particular nerve in order to selectively stimulate target efferent fibers while omitting (or minimally impacting) stimulation of other, non-target fibers and/or to selectively stimulate target efferent fibers while omitting (or minimally impacting) stimulation of other, non-target fibers.

In some examples, the care engine 2500 may implement stimulation and/or mechanical maneuvering according to a bilateral parameter 2512 in which stimulation and/or mechanical maneuvering is applied to target tissue on both sides (e.g., left and right) of the patient's body. In some such examples, the bilateral stimulation and/or mechanical maneuvering may be delivered to the same target tissue (e.g., thyroid cartilage, hyoid bone, hypoglossal nerve) on both sides of the body. However, in some examples, the bilateral stimulation and/or mechanical maneuvering may be delivered to different target tissue (e.g., thyroid cartilage, hyoid bone, hypoglossal nerve, infrahyoid muscle-innervating nerve) such as stimulating one nerve (e.g., hypoglossal nerve) or tissue on a left side of the body while stimulating another nerve (e.g., infrahyoid muscle-innervating nerve) or tissue on a right side of the body, or vice versa. In some examples, in which CSA may be treated, such as part of treating multi-type sleep apnea (e.g., both OSA and CSA), stimulation of a phrenic nerve (or diaphragm muscle) may be included in a bilateral stimulation method to implement the stimulation aspects directed to treating the central sleep apnea.

In some examples, the bilateral parameter 2512 may be implemented in a manner complementary with the alternating parameter 2532, simultaneous parameter 2534, or demand parameter 2536 of multiple function 2530, as further described below.

In some examples, the care engine 2500 may comprise a multiple function 2530 by which various care parameters may be implemented in dynamic arrangements. In some such examples, the care engine 2500 may comprise an alternating parameter 2532 by which care provided to one target tissue (e.g., hypoglossal nerve) may be alternated with care provided to at least one other target tissue (e.g., thyroid cartilage or hyoid bone). However, the alternating parameter 2532 also may be applied in combination with the bilateral parameter 2512 to apply care to the target tissue (or different target tissue) on opposite sides of the body in which care may be applied on a left side of the body and then applied on the right side of the body in an alternating manner. As used herein, applying or providing care or SDB care to target tissue may include applying stimulation and/or mechanically maneuvering the target tissue.

In some examples, the care engine 2500 may comprise a simultaneous parameter 2534 by which care may be applied simultaneously to at least two different target tissues. In some examples, the at least two different target tissues comprise two different tissues, such as the hypoglossal nerve and the thyroid cartilage. In some examples, the at least two different target tissues may comprise two different locations along the same tissue or two different fascicles of the same nerve. In some examples, the simultaneous parameter 2534 may apply stimulation per bilateral parameter 2512 simultaneously on opposite sides of the body to the same tissue or different tissue, and/or apply mechanical maneuvering simultaneously on opposite sides of the body to the same tissue (e.g., thyroid cartilage or hyoid bone).

In some examples, the care engine 2500 may comprise a demand parameter 2536 by which care may be applied to at least one target tissue on a demand basis. For example, stimulation may be applied to one nerve (e.g., hypoglossal nerve) which may be sufficient to achieve the patient metric (e.g., therapy outcome and/or usage) for most nights, for most sleeping positions (e.g., left and right lateral decubitis, prone), etc., but may become insufficient for some nights (e.g., after consuming alcohol or certain drugs which relax upper airway muscles), some sleeping positions (e.g., supine). In the latter situation, to achieve the target patient metric, via the demand parameter 2536, stimulation of a different nerve (e.g., infrahyoid muscle-innervating nerve) and/or the mechanical pulling of the thyroid cartilage and/or hyoid bone may be implemented in addition to, or instead of, stimulation of the first nerve (e.g., hypoglossal nerve) which was previously being stimulated. In some examples, the first or primary nerve being stimulated may be a nerve other than the hypoglossal nerve.

In some examples, the care engine 2500 also may further implement at least some aspects of the control portion of FIGS. 9A-13 and/or according to at least one of a closed loop parameter 2520, open loop parameter 2522, and nightly titration parameter 2524.

In some examples, the care engine 2500 comprises a closed loop parameter 2520 to deliver care based on sensed patient physiologic information and/or other information (e.g., environmental, temporal, etc.). In some such examples, via the closed loop parameter 2520 the sensed information may be used to control the particular timing of the care according to respiratory information, in which the mechanical pulling and/or stimulation pulses are timed relative to, triggered by, or synchronized with specific portions (e.g., inspiratory phase) of the patient's respiratory cycle(s). In some such examples and as previously described, the respiratory information and/or other information used with the closed loop parameter 2520 may be determined via the sensors, devices, sensing portions, as previously described in association with at least FIG. 9B.

In some examples, with or without timing care relative to sensed respiratory information, the closed loop mode (2520) may comprise delivering SDB care therapy in response to sensed disease burden, such as the average number of apnea events per a time period (e.g., AHI of average number of apnea events per hour) and/or other therapy outcome metrics (e.g., arousals, patient feedback, Epworth Sleepiness Scale (ESS) and/or other metrics). For example, for some periods of time within a nightly treatment period or over the course of several days/weeks, a patient may experience few SDB events (e.g., apnea events), such that therapy may be not delivered. However, upon the patient beginning to experience SDB at a level high enough to warrant therapy, then via the closed loop parameter 2520, mechanically pulling the thyroid cartilage and/or hyoid bone, and/or stimulation therapy may be delivered to achieve a therapy outcome and/or usage meeting a criteria per the examples of at least FIGS. 1-13.

In some examples, the care engine 2500 comprises an open loop parameter (e.g., 2522 in FIG. 14) by which SDB care (e.g., “use”) is applied without a feedback loop of sensed physiologic information. In some such examples, in an open loop mode the SDB care is applied during a treatment period without (e.g., independent of) information sensed regarding the patient's sleep quality, sleep state, respiratory phase, AHI, etc. In some such examples, in an open loop mode the SDB care is applied during a treatment period without (e.g., independent of) particular knowledge of respiratory information.

In some examples, the care engine 2500 comprises a nightly titration parameter 2524 by which an intensity of the SDB therapy may be titrated (e.g., adjusted) to be more intense (e.g., change effective length of the at least one tether, an amount of mechanically pulling, higher stimulation amplitude, greater frequency, and/or greater pulse width) or to be less intense within a nightly treatment period. However, it will be understood that the previously described examples in association with at least FIGS. 1-13 may be performed without (e.g., independent of) a nightly titration parameter 2524 and instead be based on titration according to a time period parameter of more than a day, such as supra-day time period. Accordingly, in some examples, guiding therapy per a patient metric may be implemented solely according to time period of more than a nightly treatment period.

In some such examples, the nightly titration parameter 2524 may be implemented according to at least some aspects of the example methods and/or example devices of FIGS. 1-13, for example, whether depending on the sleep state of the patient. Accordingly, in some examples, the titration parameter may be implemented as automatic titration, while in some examples, the titration parameter may be implemented via manual titration by a patient (or clinician), such as to adjust the effective length of the at least one tether as previously described. In some examples, the titration parameter may be implemented via combination of patient/manual titration and automatic titration to guide the patient in a manner complementary with manual titration.

In some such examples, such titration may be implemented at least partially based on sleep quality, which may be obtained via sensed physiologic information, in some examples. It will be understood that such examples may be employed with timing stimulation relative to (and/or in response to) sensed respiratory information (e.g., closed loop stimulation) or may be employed without timing stimulation relative to (and/or in response to) sensed respiratory information (e.g., open loop stimulation).

In some examples, at least some aspects of the titration parameter 2524 of the care engine 2500 and/or at least some aspects of titration as generally disclosed throughout FIGS. 1-14 in examples of the present disclosure may comprise (and/or may be implemented) in a manner complementary with and/or via at least some of substantially the same features and attributes as described in: (i) U.S. Pat. No. 8,938,299, issued on Jan. 20, 2015, and entitled “SYSTEM FOR TREATING SLEEP DISORDERED BREATHING”, and (ii) U.S. Patent Publication No. 2020/0147376, published on May 14, 2020, and entitled “MULTIPLE TYPE SLEEP APNEA”, each of which are hereby incorporated by reference in their entirety.

The various ranges provided herein include the stated range and any value or sub-range within the stated range. Furthermore, when “about” is utilized to describe a value, this includes, refers to, and/or encompasses variations (up to +/−10%) from the stated value.

FIG. 15 is a block diagram schematically representing an example arrangement 3100 including patient's body 3102, including example target portions 3110-3134 at which at least some example sensing element(s) and/or stimulation elements may be employed to implement at least some examples of the present disclosure.

As shown in FIG. 15, patient's body 3102 comprises a head-and-neck portion 3110, including head 3112 and neck 3114. Head 3112 comprises cranial tissue, nerves, etc., and upper airway 3116 (e.g., nerves, muscles, tissues), etc. As further shown in FIG. 15, the patient's body 3102 comprises a torso 3120, which comprises various organs, muscles, nerves, other tissues, such as but not limited to those in pectoral region 3122 (e.g., lungs 3126, cardiac 3127), abdomen 3124, and/or pelvic region 3129 (e.g., urinary/bladder, anal, reproductive, etc.). As further shown in FIG. 15, the patient's body 3102 comprises limbs 3130, such as arms 3132 and legs 3134.

It will be understood that various sensing elements (and/or stimulation elements) as described throughout the various examples of the present disclosure may be deployed within the various regions of the patient's body 3102 to sense and/or otherwise diagnose, monitor, treat various physiologic conditions such as, but not limited to the above-described examples in association with FIGS. 1-14. In some such examples, a stimulation element 3117 may be located in or near the upper airway 3116 for treating SDB (and/or near other nerves/muscles at the same or different location to treat SDB and/or other conditions) and/or a sensing element 3128 may be located anywhere within the neck 3114 and/or torso 3120 (or other body regions) to sense physiologic information for providing patient care (e.g., SDB, other).

In some examples, at least a portion of the stimulation element 3117 may comprise part of an implantable component/device, such as an IPG whether full sized or sized as a microstimulator. The implantable components (e.g., IPG, other) may comprise a stimulation/control circuit, a power supply (e.g., non-rechargeable, rechargeable), communication elements, and/or other components. In some examples, the stimulation element 3117 also may comprise at least one stimulation electrode and/or stimulation lead connected to the IPG.

Further details regarding a location, structure, operation and/or use of the sensing element 3128, external element(s) 3150, and/or stimulation element 3117 are described above in association with at least FIGS. 1-14, and in particular, at least FIGS. 10A-10E.

In some examples, any one of the implantable traction apparatuses (or a combination thereof) may be implemented as part of the example arrangement 3100 of FIG. 15 instead of, or in addition to (e.g., in complementary relation to), the stimulation element 3117, with at least some examples throughout the disclosure providing further details of such example arrangements. Moreover, at least some aspects (e.g., sensing, control, etc.) associated with an implantable traction apparatus as described in association with FIGS. 1-14 also may be implemented, in whole or part, via external element 3150 of FIG. 15.

In some examples, at least a portion of the stimulation element 3117 may comprise part of an external component/device such as, but not limited to, the external component comprising a PG (e.g., stimulation/control circuitry), power supply (e.g., rechargeable, non-rechargeable), and/other components. In some examples, a portion of the stimulation element 3117 may be implantable and a portion of the stimulation element 3117 may be external to the patient.

Accordingly, as further shown in FIG. 15, the various sensing element(s) 128 and/or stimulation element(s) 3117 implanted in the patient's body may be in wireless communication (e.g., connection 3137) with at least one external element 3150.

As further shown in FIG. 15, in some examples, the external element(s) may be implemented via a wide variety of formats such as, but not limited to, at least one of the formats 3151 including a patient support 3152 (e.g., bed, chair, sleep mat, other), wearable elements 3154 (e.g., finger, wrist, head, neck, shirt), noncontact elements 3156 (e.g., watch, camera, mobile device, other), and/or other elements 3158.

As further shown in FIG. 15, in some examples, the external element(s) 3150 may comprise one or more different modalities 3170 such as (but not limited to) a sensing portion 3171, stimulation portion 3172, power portion 3174, communication portion 3176, and/or other portion 3178. The different portions 3171, 3172, 3174, 3176, 3178 may be combined into a single physical structure (e.g., package, arrangement, assembly), may be implemented in multiple different physical structures, and/or with just some of the different portions 3171, 3172, 3174, 3176, 3178 combined together in a single physical structure.

Among other such details, in some examples the external sensing portion 3171 and/or implanted sensing element 3128 may comprise an example implementation of, and/or at least some of substantially the same features and attributes as, the examples further described above in association with FIGS. 1-14, and in particular with regard to at least FIGS. 9A-9C, 10A-10E, 11A, 11B, 13-14, respectively.

In some examples, the external stimulation portion 3172 and/or implanted stimulation element 3117 may comprise at least some of substantially the same features and attributes of at least the stimulation arrangements, as further described above in association with at least FIGS. 9A-10E, 11-14, and/or other examples throughout the present disclosure.

In some examples, the external power portion 3174 and/or power components associated with implanted stimulation element 3117 may comprise at least some of substantially the same features and attributes of at least the stimulation arrangements, as further described below in association with at least FIGS. 9A-10E, 11-14 and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 3174 may comprise a power source by which a power component of the implanted stimulation element 3117 may be recharged.

In some examples, the (wireless) communication portion 3176 (e.g., connection/link at 3137) may be implemented via various forms of radiofrequency communication and/or other forms of wireless communication, such as (but not limited to) magnetic induction telemetry, BT, BLE, NIF, near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3178 via which other aspects of implementing medical care may be embodied in external element(s) 3150 to relate to the various implanted and/or external components described above.

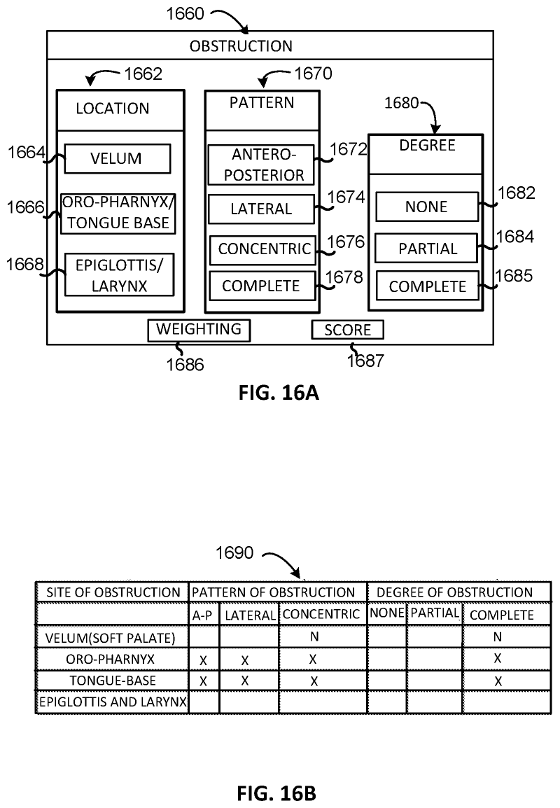

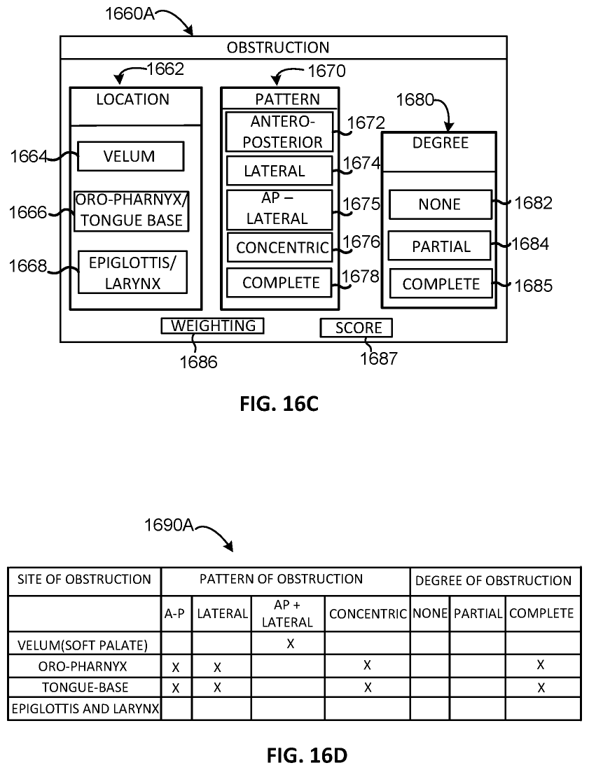

FIGS. 16A-16D are block diagrams schematically representing example devices and/or example methods relating to collapse patterns associated with upper airway patency. At least some more specific details previously described in connection with regarding FIGS. 2A-2E are further described below in relation to FIGS. 16A-16D.

FIG. 16A is a block diagram schematically representing an example sorting tool 1660 by which to sort and weigh a location, pattern, and degree of obstruction or patency. As shown in FIG. 16A, obstruction sorting tool 1660 includes functions for location detection 1662, pattern detection 1670, and degree detection 1680. In general terms, the location detection function 1662 operates to identify a site along the upper airway at which an obstruction occurs and which is believed to cause sleep disordered breathing. In one example, the location detection function 1662 includes a velum (soft palate) parameter 1664, an oropharynx-tongue base parameter 1666, and an epiglottis/larynx parameter 1668. Each respective parameter denotes an obstruction identified in the respective physiologic territories of the velum (soft palate), oropharynx-tongue base, and epiglottis which are generally illustrated for an example patient in FIG. 2A. In one aspect, these distinct physiologic territories define an array of vertical strata within the upper airway. Moreover, each separate physiologic territory (e.g., vertical portion along the upper airway) exhibits a distinct characteristic behavior regarding obstructions and associated impact on breathing during sleep. Accordingly, each physiologic territory responds differently to implantable upper airway stimulation.

With this in mind, the velum (soft palate parameter 1664 denotes obstructions taking place in the level of the region of the velum (soft palate), as illustrated in association with FIG. 2A. As previously described, FIG. 2A is a diagram including a side view schematically representing at least some anatomical features of the upper airway, as well as different sites or levels at which obstruction may occur. By determining a site or location of upper airway collapse, some example arrangements may determine whether to mechanically maneuver the thyroid cartilage and/or hyoid bone, apply stimulation via a hypoglossal nerve, via an infrahyoid muscle-innervating nerve, via a glossopharyngeal nerve, via other non-hypoglossal nerve related to upper airway patency, and/or combinations of these nerves and/or muscles including unilateral and bilateral options.

As shown in and referring back to FIG. 2A, a diagram 140 provides a side sectional view (cross hatching omitted for illustrative clarity) of a head-and-neck region 142 of a patient. In particular, an upper airway portion 150 extends from the mouth region 144 to a neck portion 155. The upper airway portion 150 includes a velum (soft palate) region 160, an oropharynx region 162, and an epiglottis region 164. The velum (soft palate) region 160 includes an area extending below sinus 161, and including the soft palate 160, approximately to the point at which tip 148 of the soft palate 146 meets a portion of tongue 147 at the back of the mouth region 144. The oropharynx region 162 extends approximately from the tip of the soft palate 146 (when in a closed position) along the base 152 of the tongue 147 until reaching approximately the tip region of the epiglottis 154. The epiglottis-larynx portion 164 extends approximately from the tip of the epiglottis 154 downwardly to a point above the esophagus 157.

As will be understood from FIG. 2A, each of these respective regions 160, 162, 164 within the upper airway correspond the respective velum parameter 1664, oropharynx parameter 1666, and epiglottis parameter 1668, respectively, of FIG. 16A.

With further reference to FIG. 16A, in general terms the pattern detection function 1670 enables detecting and determining a particular pattern of an obstruction of the upper airway. In one example, the pattern detection function 1670 includes an antero-posterior parameter 1672, a lateral parameter 1674, a concentric parameter 1676, and composite parameter 1678.

The antero-posterior parameter 1672 of pattern detection function 1670 (FIG. 16A) denotes a collapse of the upper airway that occurs in the antero-posterior orientation, as further illustrated in the diagram 210 of FIG. 2B. In FIG. 2B, arrows 211 and 212 indicate one example direction in which the tissue of the upper airway collapses, resulting in the narrowed air passage 214. FIG. 2B is also illustrative of a collapse of the upper airway in the soft palate region (160 of FIG. 2A), whether or not the collapse occurs in an antero-posterior orientation. For example, in some instances, the velum (soft palate) region (160 of FIG. 2A) exhibits a concentric (e.g., circular) pattern of collapse, as shown in diagram 220 of FIG. 2C.

The concentric parameter 1676 of pattern detection function 1670 (FIG. 16A) denotes a collapse of the upper airway that occurs in a concentric orientation, as further illustrated in the diagram 220 of FIG. 2C. In FIG. 2C, arrows 222 indicate the direction in which the tissue of the upper airway collapses, resulting in the narrowed air passage 224.

The lateral parameter 1674 of pattern detection function 1670 (FIG. 16A) denotes a collapse of the upper airway that occurs in a lateral orientation, as further illustrated in the diagram 230 of FIG. 2D. In FIG. 2D, arrows 232 and 233 indicate the direction in which the tissue of the upper airway collapses, resulting in the narrowed air passage 235.

The composite parameter 1678 of pattern detection function 1670 (FIG. 16A) denotes a collapse of the upper airway portion that occurs via a combination of the other mechanisms (lateral, concentric, antero-posterior) or that is otherwise ill-defined from a geometric viewpoint but that results in a functional obstruction of the upper airway portion.

With further reference to obstruction sorting tool 1660 of FIG. 16A, in general terms the degree detection function or module 1680 indicates a relative degree of collapse or obstruction of the upper airway portion. In some examples, the degree detection function 1680 includes a none parameter 1682, a partial collapse parameter 1684, and a complete collapse parameter 1685. In some examples, the none parameter 1682 may correspond to a collapse of 25 percent or less, while the partial collapse parameter 1684 may correspond to a collapse of between about 25 to 75%, and the complete collapse parameter 1685 may correspond to a collapse of greater than 75 percent. In some examples, at least one respiration parameter may be sensed, such as using sensing portion 2000 of FIG. 9C, that includes respiratory obstruction information, such as neural activity which is indicative of a relative degree of collapse or obstruction of the upper airway.

It will be understood that various patterns of collapse occur at different levels of the upper airway portion and that the level of the upper airway in which a particular pattern of collapse appears may vary from patient-to-patient.

In some examples, obstruction sorting tool 1660 comprises a weighting function 1686 and score function 1687. In general terms, the weighting function 1686 assigns a weight to each of the location, pattern, and/or degree parameters (FIG. 16A) as one or more those respective parameters may contribute more heavily to the patient exhibiting sleep disordered breathing or to being more responsive to implantable upper airway stimulation and/or mechanically maneuvering of tissue. More particularly, each respective parameter (e.g., antero-posterior 1672, lateral 1674, concentric 1676, composite 1678) of each respective detection modules (e.g., pattern detection function 1670) is assigned a weight corresponding to whether or not the patient is eligible for receiving implantable upper airway stimulation. Accordingly, the presence of or lack of a particular pattern of obstruction (or location or degree) will be become part of an overall score (according to score parameter 1687) for an obstruction vector indicative how likely the patient will respond to therapy via an implantable upper airway stimulation system.

FIG. 16B is diagram (e.g., chart) 1690 schematically representing an index or scoring tool to sort and weigh a location, pattern, and degree of obstruction or patency for a particular patient. Chart 1690 combines information regarding location (1662 in FIG. 16A), pattern (1670 in FIG. 16A), and degree (1680 in FIG. 16A) into a single informational grid or tool by which the obstruction is documented for a particular patient and by which appropriate stimulation settings may be determined and applied according to the various examples of the present disclosure, such as but not limited to those in association with at least FIGS. 1-15, etc.

FIGS. 16C-16D are diagrams 1660A, 1690A like the diagrams 1660, 1690 of FIGS. 16A-16B, respectively, except with FIGS. 16C-16D further addressing an AP-lateral collapse pattern, which is depicted in diagram 236 of FIG. 2E, provided as a parameter 1675 of a pattern detection function 1670 of FIG. 16C, and incorporated into the index of FIG. 16D.

As shown in FIG. 2E, this pattern comprises a combination of the anterior-posterior pattern (FIG. 2B) and the lateral pattern (FIG. 2D) with arrows 237A, 237B, 237C indicating example directions in which the tissue of the upper airway collapses, resulting in the narrowed air passage 238. The narrowed air passage 238 may comprise a triangular shape in some examples. In some examples, the AP-lateral collapse pattern at a velum/soft palate (160 in FIG. 2A, 1664 in FIG. 16C) may respond better (e.g., increase patency) to stimulation of infrahyoid-based patency tissue (e.g., infrahyoid muscle-innervating nerve(s) and/or infrahyoid strap muscle(s)) than a concentric collapse pattern having a similar severity/completeness as the AP-lateral collapse pattern at the soft palate.

Accordingly, in some examples, the information sensed and collected via at least FIGS. 16A-16D may be used to determine whether to mechanically maneuver the thyroid cartilage and/or hyoid bone, apply stimulation via a hypoglossal nerve, via a glossopharyngeal nerve, via an infrahyoid muscle-innervating nerve (including which single portion or multiple portions thereof to stimulate), via other non-hypoglossal nerves or muscles related to upper airway patency, and/or combinations of these nerves and/or muscles including unilateral and bilateral options. In some examples, other types of tissue may be stimulated, such as the phrenic nerve and/or diaphragm.

FIG. 17 schematically represents an example care engine 2700 by which at least some of substantially the same features and attributes of the examples of FIGS. 1-16D may be implemented in association with control portion 2800 (FIG. 18). In some examples, care engine 2400 may comprise at least some of substantially the same features and/or attributes as care engine 2109 of FIG. 11A.

FIG. 18 schematically represents an example control portion 2800 by which at least some of substantially the same features and attributes of the examples of FIGS. 1-16D may be implemented in association with care engine 2700 (FIG. 17). In some examples, control portion 2800 may comprise at least some of substantially the same features and/or attributes as control portion 916 of FIG. 9B, control portion 2100 of FIG. 11A, and/or control portion 2120 of FIG. 11B.

FIG. 19 schematically represents an example user interface 2840 by which at least some of substantially the same features and attributes of the examples of FIGS. 1-16D may be implemented in association with control portion 2800 (FIG. 18) and/or care engine 2700 (FIG. 17). In some examples, user interface 2840 may comprise at least some of substantially the same features and/or attributes as user interface 2240 of FIG. 12.

Various examples of the present disclosure are directed to mechanically maneuvering (e.g., pulling) the thyroid cartilage inferiorly and/or the hyoid bone anteriorly for promoting upper airway patency. In some examples, the mechanical maneuvering of thyroid cartilage and/or the hyoid bone may be used to treat sleep apnea, such as OSA. In some examples, the mechanical manipulating may be used in addition to other SDB treatment for treating OSA or multiple type sleep apnea, such as OSA and CSA. For example, the thyroid cartilage inferiorly and/or the hyoid bone may be selectively mechanically maneuvered at different or same times as applying electrical stimulation to tissue and/or activating an external breathing therapy device.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1-85. (canceled)

86. A method, comprising:
promoting patency of an upper airway of a patient via mechanically maneuvering at least one of:
thyroid cartilage inferiorly; and
hyoid bone anteriorly.

87. The method of claim 86, wherein mechanically maneuvering the at least one of the thyroid cartilage inferiorly and the hyoid bone anteriorly causes at least one of an increase of and maintaining of patency of the upper airway.

88. The method of claim 86, wherein mechanically maneuvering the at least one of the thyroid cartilage inferiorly and the hyoid bone anteriorly causes the upper airway to stretch inferiorly to stiffen and reduce collapsibility.

89. The method of claim 86, wherein mechanically maneuvering the at least one of the thyroid cartilage and the hyoid bone comprises at least one of electromechanically maneuvering the thyroid cartilage inferiorly and electromechanically maneuvering the hyoid bone anteriorly.

90. The method of claim 86, wherein promoting patency of the upper airway comprises pulling the hyoid bone via the mechanically maneuvering of the hyoid bone anteriorly.

91. The method of claim 86, wherein mechanically maneuvering the at least one of the thyroid cartilage inferiorly and the hyoid bone anteriorly causes tissue at least partially S/N New Non-Provisional Application defining the upper airway to compress thereby causing an increase in a cross-sectional area of the upper airway.

92. The method of claim 86, further comprising adjusting an amount of the maneuvering over time for the patient.

93. The method of claim 86, wherein mechanically maneuvering the thyroid cartilage comprises adjusting an effective length of at least one tether of an implantable traction apparatus to perform at least one of:
pulling the thyroid cartilage inferiorly; and
pulling the hyoid bone anteriorly.

94. The method of claim 93, wherein the implantable traction apparatus comprises at least one implantable winch.

95. The method of claim 94, wherein the at least one implantable winch operates by one of:
solely mechanically; and
electromechanically.

96. The method of claim 93, wherein the adjustment to the effective length is revised over time.

97. The method of claim 93, further comprising:
monitoring a therapy outcome during a treatment period; and
adjusting the effective length based on the therapy outcome.

98. The method of claim 93, further comprising anchoring the at least one tether to non-nerve tissue to which upper airway-dilating muscles attach.

99. The method of claim 98, further comprising at least one of reducing an amount of retraction and releasing the at least one tether in response to tension on the at least one tether that is outside a threshold.

100. An implantable traction apparatus, comprising:
at least one component configured to mechanically maneuver at least one of the thyroid cartilage inferiorly and the hyoid bone anteriorly.

101. The apparatus of claim 100, wherein the at least one component is an implantable winch comprising:
at least one tether; and
at least one actuator coupled to the at least one tether and configured to cause the mechanical maneuvering.

102. The apparatus of claim 101, wherein the at least one tether comprises a first anchor element on a first end configured to attach to non-nerve tissue and an opposite second end is coupled to the at least one actuator and the apparatus further comprising a second anchor element on a portion of the implantable winch configured to attach to other non-nerve tissue that is one of inferior and anterior to the non-nerve tissue.

103. The apparatus of claim 102, wherein the portion of the implantable winch comprises one of:
the at least one actuator; and
at least one cable coupled to the at least one actuator.

104. The apparatus of claim 100, wherein the at least one component comprises a first implantable winch and a second implantable winch, each of the first and second implantable winches comprise:
at least one tether; and
an actuator coupled to the at least one tether and configured cause the mechanical maneuvering.

105. The apparatus of claim 100, wherein the at least one component comprises at least one scissor extension arm configured to attach to non-nerve tissue and to change between an expanded state and a condensed state.
